(19) United States
(12) Patent Application Publication
(10) Pub. No.: US 2025/0121047 A1
(43) Pub. Date: Apr. 17, 2025

(54) CHIMERIC VIRUS EXPRESSING PORCINE PRODUCTIVE AND RESPIRATORY SYNDROME VIRUS-DERIVED PEPTIDE AND VACCINE COMPOSITION COMPRISING SAME

(71) Applicants: BioPoA, Inc., Hwaseong-si (KR); REPUBLIC OF KOREA(Animal and Plant Quarantine Agency), Gimcheon-si (KR)

(72) Inventors: Sang Ho CHA, Cheonan-si (KR); Seok-Jin KANG, Gimcheon-si (KR); Suhwa YOU, Gimcheon-si (KR); Bang-Hun HYUN, Gimcheon-si (KR); Sun Hee CHO, Seongnam-si (KR); Changhoon PARK, Seoul (KR)

(21) Appl. No.: 18/724,753

(22) PCT Filed: Jan. 28, 2022
(86) PCT No.: PCT/KR2022/001653

§ 371 Date: Dec. 20, 2024

(30) Foreign Application Priority Data
Dec. 30, 2021 (KR) 10-2021-0193458
Dec. 30, 2021 (KR) 10-2021-0193459

(51) Int. Cl. A61K 39/12; A61K 39/00; A61P 37/04; C07K 14/005; C12N 7/00

(52) U.S. Cl. CPC A61K 39/12; A61P 37/04; C07K 14/005; C12N 7/00; A61K 2039/5256; A61K 2039/575; C12N 2720/12322; C12N 2770/10022; C12N 2770/10034; C12N 2770/10052; C12N 2770/10071

(57) ABSTRACT

Provided are a chimeric virus expressing a porcine reproductive and respiratory syndrome virus (PRRSV)-derived peptide and a use thereof as a vaccine. The chimeric virus has an excellent immune stimulating effect and is useful as a vaccine that can effectively protect against PRRSV by suppressing viral amplification in target cells.

### TECHNICAL FIELD

Chimeric viruses expressing a Porcine Reproductive and Respiratory Syndrome Virus (PRRSV)-derived peptide (T cell epitope) and uses thereof as a vaccine are provided. The chimeric viruses are useful as a vaccine which has an excellent immunostimulatory effect and can effectively protect against it by inhibiting virus amplification in target cells.

### BACKGROUND ART

Porcine Reproductive and Respiratory Syndrome (hereinafter, PRRS) corresponds to an infectious disease that causes the greatest damage to the domestic pig farming industry with porcine circovirus associated diseases and foot-and-mouth disease. PRRS causes reproductive disorders such as reproduction inability, miscarriage or premature birth, and stillbirth of pregnant sows, and causes respiratory symptoms such as sneezing, fever and the like in suckling pigs and finishing pigs. In general, secondary infection of bacteria and the like causes severe respiratory symptoms after morbidity into a virus, but in chronically infected cases, a decrease in body weight gain and an increase in mortality occur without characteristic clinical symptoms.

This viral disease was first discovered in the United States in 1987, and then, discovered in Europe, and identified in Asia in the early 1990s. Until now, PRRS has properties of endemic disease in pig farming countries, and has spread in the world, causing enormous economic losses every year.

A causative pathogen of PRRS is a PRRS virus belonging to the genus Arterivirus, the family Arteriviridae and the order Nidovirales. The PRRS virus has positive-sense single stranded RNA genome, and the size is about 15.4 kilobases. The PRRS virus genome has 9 ORFs (Conzelmann et al., 1993; Meulenberg et al., 1993). Among them, ORF1a and ORF1b encoding Non-Structural Protein (NSP) account for about 80% of the virus genome (Bautista et al., 2002; Meulenberg et al., 1993; Snijder and Meulenberg, 1998, 2001). NSP1-alpha, NSP-1 beta, NSP2 to NSP8 among the non-structural protein is known to be positioned at ORF1b. GP2, GP3, GP4, GP5 which are glycosylated structural proteins and non-glycosylated membrane (M) protein and nucleocapsid (N) protein are encoded by ORF2-7 accounting for the remaining 20%. Minor structural proteins, GP2, GP3, and GP4 from a heterotrimer, which act when a virus invades a host cell, and major structural proteins, GP5 and M form a heterodimer, which act to increase infectivity of the virus.

The PRRS virus is highly variable due to the nature of RNA viruses, and therefore, there are many differences between the viruses. The PRRS virus is largely divided into North American and European types. There are Type I representing the European type (Lelystad virus, LV) and Type II representing the North American virus strain ATCC VR2332 (See GenBank accession number AY150564 for the genome sequence of VR2332) (Murtaugh et al., Arch Virol. 1995; 140:1451-1460).

It has been known that there are gene differences up to 40% at maximum between the North American type and European type, and they do not cross-protect against each other. In addition, there are many cases where cross-protection is not achieved even between mutant strains belonging to the same type (Meng, X. J. et al., 2000). Due to this, vaccines based on standard mutant strains for each, but the cross-protection ability is not good, so it cannot effectively prevent PRRS. To overcome this, various attempts are being made to produce a vaccine with safety, immunogenicity, and protective activity effectively.

Due to the severity of such a disease, since the Porcine Reproductive and Respiratory Syndrome Virus (PRRSV), which is a pathogen of Porcine Reproductive and Respiratory Syndrome (PRRS), was discovered, a lot of efforts have been invested to develop a method for prevention against this virus for about 20 years, but no effective prevention method and management method have not been developed yet. Various vaccines such as inactivated vaccines and attenuated live vaccines have been developed to control the Porcine Reproductive and Respiratory Syndrome Virus (PRRSV), but only attenuated vaccines with guaranteed infectivity were found to induce a preventive effect at a satisfactory level. However, as the Porcine Reproductive and Respiratory

Syndrome Virus (PRRSV) are present genetically in great diversity, cross-immunity between various virus types is absent, so it is difficult to prevent various Porcine Reproductive and Respiratory Syndrome viruses (PRRSVs) with one vaccine. In addition, currently, attenuated vaccines can be produced only through serial passages of 100˜200 or more passages in cell lines of other animal species, so there are problems that the development period is long and it is difficult to guarantee efficacy and safety. Due to this, vaccines based on standard mutant strains for each were produced, but the cross-protection ability is not good, so they do not effectively prevent PRRS.

In order to overcome this, various attempts are being made to produce a vaccine with safety, immunogenicity, and protective activity effectively (Application No. 10-2011-7004020, Title of the Invention: Vaccine against highly pathogenic Porcine Reproductive and Respiratory Syndrome (HP PRRS)).

### DISCLOSURE

### Technical Problem

The present invention discovers a peptide which has low pathogenicity and high stability, and can activate cellular immunity using a reverse genetics technique, and provides a vaccine strain expressing the same.

One embodiment provides a T cell epitope-expressing chimeric virus equipped with a Porcine Reproductive and Respiratory Syndrome Virus (PRRSV)-derived T cell epitope.

Another embodiment provides a vaccine composition comprising the chimeric virus.

Other embodiment provides a method of preparation of the chimeric virus.

In one embodiment, the chimeric virus is genome, which comprises
(1) a nucleic acid sequence of ORF1a and ORF1b derived from a PRRS type II virus or a nucleic acid sequence having sequence homology of 70% or more with the nucleic acid sequence while maintaining functional equivalence;
(2) a nucleic acid sequence encoding T cell epitope-EP7 derived from a porcine reproductive and respiratory syndrome virus or a nucleic acid sequence having sequence homology of 70% or more with the nucleic acid sequence while maintaining functional equivalence and/or a nucleic acid sequence encoding T cell epitope-EP8 derived from porcine reproductive and respiratory syndrome virus or a nucleic acid sequence having sequence homology of 70% or more with the nucleic acid sequence while maintaining functional equivalence; and (3) a nucleic acid sequence of ORF2 to ORF7 regions of BP2017-2 virus of accession number KCTC 13393BP or a nucleic acid sequence having sequence homology of 70% or more with the nucleic acid sequence while maintaining functional equivalence.

The nucleic acids (1), (2) and (3) may be arranged in the 5′->3′ direction in sequence.

The nucleic acid sequence of ORF1a and ORF1b derived from a PRRS type II virus may be represented by SEQ ID NO: 1 or SEQ ID NO: 2.

The nucleic acid sequence encoding T cell epitope-EP7 derived from a porcine reproductive and respiratory syndrome virus may be a nucleic acid sequence encoding the amino acids of SEQ ID NO: 3, and more specifically, it may be represented by SEQ ID NO: 4.

The nucleic acid sequence encoding T cell epitope-EP8 may be a nucleic acid sequence encoding the amino acids of SEQ ID NO: 12, and more specifically, it may be represented by SEQ ID NO: 10.

The nucleic acid sequence of ORF2 to ORF7 regions of the BP2017-2 virus may be represented by SEQ ID NO: 5.

### Technical Solution

The present invention relates to a chimeric virus of a Porcine Reproductive and Respiratory Syndrome (PRRS) virus which can be used as a vaccine. The chimeric virus of PRRSV of the present invention is attenuated than a parent strain, so it has low pathogenicity and high stability, and improves secretion of neutralizing antibodies capable of cross-immunity, thereby significantly enhancing porcine immunity. Therefore, it can be used as an effective vaccine for prevention and treatment of PRRS disease.

Hereinafter, the present invention will be described in more detail.

### DEFINITION OF TERMS

“Attenuated virus” used in the present description refers to an avirulent virus capable of causing an immune response in a target mammal without causing clinical signs of PRRS disease, and also refers to one of which incidence of clinical signs is reduced in an animal infected with an attenuated virus and not administered with the attenuated virus, or the severity of the signs is reduced compared to a “control” animal infected with a non-attenuated PRRS virus. In this situation, the term of “reduction/reduced” means a reduction of at least 10%, preferably, 25%, more preferably, 50%, and most preferably 100% or more, compared to the control group as defined above.

“Vaccine composition” used in the present description may be a PRRS chimeric virus or any immunogenic fragment or fraction thereof, preferably, an attenuated PRRS chimeric virus, for example, the PRRS chimeric virus of the present invention. This causes “immunological response” of a host as a cell and/or antibody-mediated immune response against PRRSV. It is preferable that the vaccine composition can give preventive immunity against PRRSV infection and clinical signs related thereto.

“Immune response” used in the present description refers to any cell- and/or antibody-mediated immune response against a chimeric virus or vaccine which is administered to an animal administered with the chimeric virus of PRRS of the present invention, or a vaccine composition comprising the same. In common, “immune response” comprises at least one of the following effects, but not limited thereto: production or activation of antigens comprised in the corresponding composition or vaccine or an antibody, a B cell, a helper T cell, a repressor T cell, and/or a cytotoxic T cell and/or a y8 T cell, which are specifically induced against the antigens. It is preferable that the host shows a therapeutic or preventive immunological response so that resistance to new infections is improved and/or the clinical severity of disease is reduced compared to a control group not administered with the immunogenic composition or vaccine. Such prevention may be verified by absence of symptoms related to host infections and a reduction of the frequency or severity including it as described above at most.

The terms used in the present description, “pigs”, “pig” and “piglets” may be interchangeably used with mutually equivalent meanings.

The term “inoculates a vaccine” means administering the chimeric virus of PRRS described in the present description or a vaccine comprising the same before exposed to PRRS disease.

The term “prevent” or “prevention” refers to a reduction in the clinical incidence of PRRS, severity of signs or frequency, as a result of administration of the PRRSV virus of the present invention or a vaccine composition comprising the same. The reduction in the severity or frequency may be a result of comparing an animal or animal group not administered with the chimeric virus of PRRSV of the present invention or the vaccine composition comprising the same. The animal may be preferably a pig.

The nucleic acid sequence of the present invention is described based on DNA nucleotides for convenience, and when the type of polynucleotide is RNA (for example, genome of PRRS virus) refers to a sequence in which all or some of thymine (T) is substituted with uracil (U) in the base sequence.

In the present description, that a polynucleotide (“may be interchangeably used with “gene”) or polypeptide “comprises a specific nucleic acid sequence or amino acid sequence or consists of or is presented by a specific nucleic acid sequence or amino acid sequence” may mean that the polynucleotide or polypeptide essentially comprises the specific nucleic acid sequence or amino acid sequence, and it may be interpreted as including “substantially equivalent sequences” in which a mutation (deletion, substitution, modification and/or addition) is added to the specific nucleic acid sequence or amino acid sequence (or not excluding the mutation) within a range that maintains the original function and/or desired function of the polynucleotide or polypeptide.

In one embodiment, that a polynucleotide or polypeptide “comprises a specific nucleic acid sequence or amino acid sequence, or consists of or is represented by the sequence” may mean that the polynucleotide or polypeptide (i) essentially comprises the specific nucleic acid sequence or amino acid sequence, or (ii) consists of an amino acid sequence having identity of 70% or more, 80% or more, 85% or more, 90% or more, 91% or more, 92% or more, 93% or more, 94% or more, 95% or more, 96% or more, 97% or more, 98% or more, 99% or more, 99.5% or more, or 99.9% or more to the specific nucleic acid sequence or amino acid sequence or essentially comprises the same, and maintains the original function and/or desired function.

In the present description, the term “identity” refers to a degree of corresponding with a given nucleic acid sequence or amino acid sequence, and may be expressed as a percentage. In case of the identity to a nucleic acid sequence, for example, it may be determined using algorithm BLAST by a literature (See: Karlin and Altschul, Pro. Nat1. Acad. Sci. USA, 90, 5873, 1993) or FASTA by Pearson (See: Methods Enzymol., 183, 63, 1990). Based on this algorithm BLAST, programs called BLASTN or BLASTX have been developed (See: http://www.ncbi.nlm.nih.gov).

In the present description, the term “nucleic acid sequence or base sequence” may mean sequence information of bases comprised in nucleotides of nucleic acid molecules (for example, oligonucleotide or polynucleotide) comprising at least two nucleotides (DNA or RNA) or a nucleic acid molecule having the base sequence.

In the present description, the term “construction” refers to inserting a gene into a cell, thereby restoring infectivity of a virus produced by transduction into the cell to exhibit a denaturing effect in the cell.

### Chimeric Virus Mutant Strain

One embodiment provides an attenuated chimeric virus mutant strain of a Porcine Reproductive and Respiratory Syndrome Virus (PRRSV). Another embodiment provides a polynucleotide, and the polynucleotide may constitute genome of the chimeric virus mutant strain.

As described in the present description, the chimeric virus may refer to that a part of genome of any one species of Porcine Reproductive and Respiratory Syndrome Viruses (PRRSVs) is substituted (replaced) with the corresponding genome part of another species of PRRSV. More specifically, the chimeric virus may be a PRRS type II virus, for example, a chimeric virus of PRRSV having genome in which ORF1a and ORF1b regions (NSP1) derived from LMY parent strain (Accession No. GenBank accession no. DQ473474.1) or LMY ver2 virus of accession number KCTC 13394BP, which is LMY parent strain PRRSV, and ORF2 to ORF7 regions of the BP2017-2 virus of accession number KCTC 13393BP, which is PRRSV, are fused.

The chimeric virus mutant strain may refer to that a mutation is applied to the chimeric virus of PRRSV described above, more specifically, that a mutation is applied to express T cell epitope-EP7 derived from PRRSV, and this is named a chimeric virus mutant strain expressing EP7 or PRRSV-EP7 chimeric virus.

The chimeric virus mutant strain expressing EP7 may be used as a vaccine strain which stimulates porcine PBMCs (peripheral blood mononuclear cells) infected with a PRRS virus (type I and/or type II) to induce interferon-gamma (IFN-gamma) and inhibits virus amplification in a target cell to effectively prevent it.

In addition, the T cell epitope-EP7 is a part of the amino acid sequence of the membrane protein of PRRSV, and it has been confirmed that when it stimulates PBMCs isolated from pigs infected with PRRSV, interferon-gamma is induced, and interferon-gamma at a high level is induced in PBMCs when inoculated into the pigs infected with PRRSV.

The chimeric mutant strain may be that a mutation is applied to the chimeric virus of PRRSV described above, more specifically, that a mutation is applied to express T cell epitope-EP8 derived from PRRSV, and this is named a chimeric virus mutant strain expressing EP8 or PRRSV-EP8 chimeric virus.

The chimeric virus mutant strain expressing EP8 may be used as a vaccine strain which stimulates porcine PBMCs (peripheral blood mononuclear cells) infected with a PRRS virus (type I and/or type II) to induce interferon-gamma (IFN-gamma) and inhibits virus amplification in a target cell to effectively prevent it.

Furthermore, the T cell epitope-EP8 is a part of the amino acid sequence of the membrane protein of PRRSV, and it has been confirmed that when it stimulates PBMCs isolated from pigs infected with PRRSV, interferon-gamma is induced, and interferon-gamma at a high level is induced in PBMCs when inoculated into the pigs infected with PRRSV.

One embodiment provides a polynucleotide represented by Structural formula 1:

[Structural formula 1]
5′-[X]-[Y]-[Z]-3′

In the formula, 0 to 20,000 nucleotides may be further comprised between [X] and [Y], and [Y] and [Z], but not limited thereto.

In the formula, [X] may comprise a nucleic acid sequence of NSP1 gene (NSP1-alpha gene and NSP1-beta gene; ORF1a and ORF1b) of a PRRS type II virus, for example, LMY parent strain (Accession No. GenBank accession no. DQ473474.1) or LMY ver2 virus with accession number KCTC 13394BP or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% [symbol] ; hereinafter, applied in the same way) within a range of maintaining equivalent functions to the nucleic acid sequence, and for example, the nucleic acid sequence of the NSP1 gene of the LMY ver2 virus may be represented by SEQ ID NO: 2, and the nucleic acid sequence of the NSP1 gene of the LMY parent strain may be represented by SEQ ID NO: 1.

The [Y] may be a nucleic acid sequence encoding T cell epitope-EP7 (LLAFSITYTPVMIYALKVSRGRLLGL; SEQ ID NO: 3), which is a PRRSV-derived membrane protein, or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the nucleic acid sequence while maintaining functional equivalency thereto.

Specifically, the EP7-encoding nucleic acid sequence may be the nucleic acid sequence of SEQ ID NO: 4 or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the nucleic acid sequence of SEQ ID NO: 4 while maintaining functional equivalency thereto.

In addition, the [Y] may be a modified EP7-encoding nucleic acid sequence. The modified EP7-encoding nucleic acid sequence, may further comprise at least one selected from the group consisting of AscI restriction enzyme recognition site (GGCGCGCC; SEQ ID NO: 8), Kozak sequence (GCCACC; SEQ ID NO: 13) and a GTTCCGTGGCAACCCCTATAACCAGAGTTTCAGCGGAACA; SEQ ID NO: 14), in addition to the EP7-encoding nucleic acid sequence. In one embodiment, the AscI restriction enzyme recognition site may be comprised at both ends of the EP7-encoding nucleic acid sequence, respectively. In one embodiment, the Kozak sequence and/or transcription regulatory sequence may be each independently comprised at the 5′ end and/or 3′ end of the EP7-encoding nucleic acid sequence, for example, at the 5′ end for the Kozak sequence, and the 3′ end for the transcription regulatory sequence (TRS6).

In one specific embodiment, the [Y] may comprise an EP7-encoding nucleic acid sequence (e.g., SEQ ID NO: 4), Kozak sequence positioned at the 5′ end of the EP7-encoding nucleic acid sequence (e.g., SEQ ID NO: 13), TRS6 positioned at the 3′ end of the EP7-encoding nucleic acid sequence (e.g., SEQ ID NO: 14), and the AscI restriction enzyme recognition site positioned at the 5′ end of the Kozak sequence and the 3′ end of the TRS6, respectively (SEQ ID NO: 8), and specifically, it may be the nucleic acid sequence of SEQ ID NO: 20 or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the nucleic acid sequence of SEQ ID NO: 20 while maintaining functional equivalency thereto.

The [Y] may be a nucleic acid sequence encoding T cell epitope-EP8 (LWGVYSAIETWKFITSRCRLCLLGRKYILAPAHHVESA; SEQ ID NO: 12), which is a PRRSV-derived membrane protein, or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the nucleic acid sequence while maintaining functional equivalency thereto. Specifically, the EP8-encoding nucleic acid sequence may be the nucleic acid sequence of SEQ ID NO: 10 or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the nucleic acid sequence of SEQ ID NO: 10 while maintaining functional equivalency thereto.

In addition, the [Y] may be a modified EP8-encoding nucleic acid sequence. The modified EP8-encoding nucleic acid sequence, may further comprise at least one selected from the group consisting of AscI restriction enzyme recognition site (GGCGCGCC; SEQ ID NO: 8), Kozak sequence (GCCACC; SEQ ID NO: 13) and a GTTCCGTGGCAACCCCTATAACCAGAGTTTCAGCGGAACA; SEQ ID NO: 14), in addition to the EP8-encoding nucleic acid sequence. In one embodiment, the AscI restriction enzyme recognition site may be comprised at both ends of the EP8-encoding nucleic acid sequence, respectively. In one embodiment, the Kozak sequence and/or transcription regulatory sequence may be each independently comprised at the 5′ end and/or 3′ end of the EP8-encoding nucleic acid sequence, for example, at the 5′ end for the Kozak sequence, and the 3′ end for the transcription regulatory sequence (TRS6).

In one specific embodiment, the [Y] may comprise an EP8-encoding nucleic acid sequence (e.g., SEQ ID NO: 10), Kozak sequence positioned at the 5′ end of the EP8-encoding nucleic acid sequence (e.g., SEQ ID NO: 13), TRS6 positioned at the 3′ end of the EP8-encoding nucleic acid sequence (e.g., SEQ ID NO: 14), and the AscI restriction enzyme recognition site positioned at the 5′ end of the Kozak sequence and the 3′ end of the TRS6, respectively (SEQ ID NO: 8), and specifically, it may be the nucleic acid sequence of SEQ ID NO: 21 or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the nucleic acid sequence of SEQ ID NO: 21 while maintaining functional equivalency thereto.

The [Z] may be a gene nucleic acid sequence of ORF2 to ORF7 regions of the BP2017-2 virus with accession number KCTC 13393BP or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) within a range of maintaining equivalent functions to the nucleic acid sequence.

As used in the present description, the “equivalent function” may mean a qualitatively (activity) and/or quantitatively (level) same or similar function to the desired function. For example, the amino acid sequence of the protein encoded by the gene may be same as the amino acid sequence of the protein encoded by the wild-type gene.

In one embodiment, the [X] may comprise a nucleic acid sequence encoding NSP1 derived from a PRRS type II virus, for example a nucleic acid sequence of the NSP1 gene of the LMY parent strain (Accession No. GenBank accession no. DQ473474.1.) consisting of the nucleic acid sequence of SEQ ID NO: 1, or in the nucleic acid sequence, all kinds of point mutations (silent mutations) occurring within a range of maintaining the amino acid sequence of the NSP1 protein encoded by the NSP1 gene identically, and for example, the mutation may be freely selected and used within a target range for lowering a CPB (Codon Pair Bias) value of the NSP1 gene. The CPB (Codon Pair Bias) may mean a bias caused by interaction of viral gene codons when arranged in pairs, and may be quantified using a computer algorithm, and when the CPB value is reduced (deoptimized), the proliferation of the virus is reduced and attenuated (Virus Attenuation by Genome-Scale Changes in Codon Pair Bias, Science, 2008, J. Robert Coleman et al.), so by substituting some base sequences with a high CPB value with base sequences with a low CPB value, or silent mutation according to Codon Pair Deoptimization principle, an attenuated mutant strain of LMU used in preparation of the chimeric virus provided in the present description may be produced.

In one specific embodiment, for attenuation, bases substituted among the base sequence of the gene encoding NSP1 of the LMY parent strain may be selected using known SAVE (Synthetic Attenuated Virus Engineering) program. Specifically, the [X] region in the present description may be a base sequence in which some or all are selected and substituted to deoptimize them in a region showing relatively higher Codon Pair Bias (CPB) by analyzing a NSP1 region with genetically high safety in the genome of LMY, which is the parent strain.

For example, the [X] may comprise (i) the nucleic acid sequence of the NSP1 gene represented by SEQ ID NO: 1, or (ii) a mutation in which one or more, 25 or more, 66 or more, 80 or more or all 91 positions selected from the group consisting of the 222th position, 225th position, 237th position, 240th position, 252th position, 306th position, 309th position, 312th position, 315th position, 324th position, 327th position, 330th position, 333th position, 336th position, 339th position, 342th position, 345th position, 357th position, 363th position, 366th position, 378th position, 379th position, 381th position, 393th position, 396th position, 543th position, 546th position, 549th position, 555th position, 558th position, 561th position, 573th position, 579th position, 582th position, 588th position, 612th position, 618th position, 621th position, 627th position, 633th position, 639th position, 654th position, 673th position, 675th position, 678th position, 681th position, 684th position, 705th position, 708th position, 729th position, 735th position, 738th position, 741th position, 744th position, 747th position, 771th position, 786th position, 789th position, 792th position, 810th position, 825th position, 828th position, 838th position, 840th position, 846th position, 849th position, 858th position, 867th position, 879th position, 882th position, 885th position, 891th position, 900th position, 903th position, 906th position, 924th position, 936th position, 939th position, 948th position, 954th position, 963th position, 966th position, 1026th position, 1029th position, 1038th position, 1047th position, 1053th position, 1066th position, 1068th position, 1086th position, and 1110th position, are substituted in the nucleic acid sequence of SEQ ID NO: 1.

For example, the [X] may comprise (i) the nucleic acid sequence of the NSP1 gene represented by SEQ ID NO: 1, or (ii) a mutation in which one or more, 25 or more, 66 or more, 80 or more or all 91 positions selected from the group consisting of mutation in which G at the 222th position is substituted with C, mutation in which C at the 225th position is substituted with A, mutation in which T at the 237th position is substituted with C, mutation in which A at the 240th position is substituted with T, mutation in which T at the 252th position is substituted with C, mutation in which A at the 306th position is substituted with C, mutation in which T at the 309th position is substituted with C, mutation in which G at the 312th position is substituted with A, mutation in which C at the 315th position is substituted with A, mutation in which T at the 324th position is substituted with C, mutation in which C at the 327th position is substituted with G, mutation in which G at the 330th position is substituted with A, mutation in which C at the 333th position is substituted with T, mutation in which C at the 336th position is substituted with G, mutation in which T at the 339th position is substituted with C, mutation in which A at the 342th position is substituted with T, mutation in which T at the 345th position is substituted with A, mutation in which A at the 357th position is substituted with A, mutation in which T at the 363th position is substituted with A, mutation in which T at the 366th position is substituted with C mutation in which C at the 378th position is substituted with T, mutation in which C at the 379th position is substituted with A, mutation in which C at the 381th position is substituted with G, mutation in which T at the 393th position is substituted with C, mutation in which T at the 396th position is substituted with A, mutation in which T at the 543th position is substituted with C, mutation in which T at the 546th position is substituted with C, mutation in which C at the 549th position is substituted with A, mutation in which T at the 555th position is substituted with C, mutation in which T at the 558th position is substituted with C, mutation in which T at the 561th position is substituted with A, mutation in which C at the 573th position is substituted with T, mutation in which T at the 579th position is substituted with C, mutation in which G at the 582th position is substituted with T, mutation in which T at the 588th position is substituted with C, mutation in which T at the 612th position is substituted with C, mutation in which G at the 618th position is substituted with C, mutation in which T at the 621th position is substituted with C, mutation in which A at the 627th position is substituted with T, mutation in which T at the 633th position is substituted with C, mutation in which T at the 639th position is substituted with G, mutation in which C at the 654th position is substituted with T, mutation in which C at the 673th position is substituted with T, mutation in which C at the 675th position is substituted with A, mutation in which C at the 678th position is substituted with T, mutation in which C at the 681th position is substituted with G, mutation in which C at the 684th position is substituted with G, mutation in which G at the 705th position is substituted with C, mutation in which C at the 708th position is substituted with T, mutation in which A at the 729th position is substituted with C, mutation in which T at the 735th position is substituted with C, mutation in which G at the 738th position is substituted with T, mutation in which T at the 741th position is substituted with C, mutation in which T at the 744th position is substituted with C, mutation in which T at the 747th position is substituted with A, mutation in which T at the 771th position is substituted with C, mutation in which T at the 786th position is substituted with C, mutation in which C at the 789th position is substituted with T, mutation in which T at the 792th position is substituted with G, mutation in which C at the 810th position is substituted with T, mutation in which T at the 825th position is substituted with C, mutation in which C at the 828th position is substituted with T, mutation in which T at the 838th position is substituted with C, mutation in which G at the 840th position is substituted with C, mutation in which C at the 846th position is substituted with G, mutation in which G at the 849th position is substituted with A, mutation in which A at the 858th position is substituted with G, mutation in which T at the 867th position is substituted with C, mutation in which T at the 879th position is substituted with C, mutation in which C at the 882th position is substituted with G, mutation in which C at the 885th position is substituted with T, mutation in which C at the 891th position is substituted with T, mutation in which T at the 900th position is substituted with C, mutation in which C at the 903th position is substituted with A, mutation in which T at the 906th position is substituted with C, mutation in which G at the 924th position is substituted with T, mutation in which T at the 936th position is substituted with C, mutation in which T at the 939th position is substituted with C, mutation in which A at the 948th position is substituted with C, mutation in which A at the 954th position is substituted with C, mutation in which A at the 963th position is substituted with T, mutation in which T at the 966th position is substituted with C, mutation in which A at the 1026th position is substituted with G, mutation in which G at the 1029th position is substituted with C, mutation in which C at the 1038th position is substituted with T, mutation in which T at the 1047th position is substituted with C, mutation in which T at the 1053th position is substituted with C, mutation in which A at the 1066th position is substituted with C, mutation in which A at the 1068th position is substituted with C, mutation in which C at the 1086th position is substituted with T, and mutation in which T at the 1110th position is substituted with C, in the nucleic acid sequence of SEQ ID NO: 1.

The attenuated mutant strain of LMY prepared by introducing the mutation as above may be LMY ver2 virus with accession number 13394BP, and the LMY ver2 virus may have the nucleic acid sequence of SEQ ID NO: 2 as the gene encoding NSP1 protein.

Therefore, the [X] may be the nucleic acid sequence of the NSP1 gene (SEQ ID NO: 2) of the LMY ver2 virus with accession number 13394BP, the nucleic acid sequence of the NSP1 gene (SEQ ID NO: 1) of the LMY parent strain, or a nucleic acid sequence having sequence identity of 70% or more, 75% or more, 80% or more, 85% or more, 90% or more, 95% or more, 96% or more, 97% or more, 98% or more, 99% or more, or 99.5% or more within a range of maintaining equivalent functions to the nucleic acid sequence.

The mutant strain sequence of LMY provided in the present description, for example, the sequence of LMY ver2 (SEQ ID NO: 2) may have lower a Codon Pair Bias (CPB) compared to the wild-type region, and the wild-type LMY region may consist of the nucleic acid sequence of SEQ ID NO: 1, but not limited thereto.

In one embodiment, the [Y] may be a nucleic acid sequence encoding T cell epitope-EP7 derived from porcine reproductive and respiratory syndrome virus of the amino acid sequence LLAFSITYTPVMIYALKVSRGRLLGL (SEQ ID NO: 3), for example, the nucleic acid sequence of SEQ ID NO: 4, or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the nucleic acid sequence of SEQ ID NO: 4 while maintaining functional equivalency thereto.

In other embodiment, the [Y] may further comprise at least one selected from the group consisting of AscI restriction enzyme recognition site SEQ ID NO: 8), Kozak sequence (SEQ ID NO: 13) and TRS6 (SEQ ID NO: 14), and for example, it may be the nucleic acid sequence of SEQ ID NO: 20 or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the sequence of SEQ ID NO: 20 while maintaining functional equivalency thereto.

In one embodiment, the [Y] may be a nucleic acid sequence encoding T cell epitope-EP8 derived from porcine reproductive and respiratory syndrome virus of the amino acid sequence LWGVYSAIETWKFITSRCRLCLLGRKYILAPAHHVESA (SEQ ID NO: 12), for example, the nucleic acid sequence of SEQ ID NO: 10 or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the nucleic acid sequence of SEQ ID NO: 10 while maintaining functional equivalency thereto.

In other embodiment, the [Y] may further comprise at least one selected from the group consisting of AscI restriction enzyme recognition site SEQ ID NO: 8), Kozak sequence (SEQ ID NO: 13) and TRS6 (SEQ ID NO: 14), and for example, it may be the nucleic acid sequence of SEQ ID NO: 21 or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the sequence of SEQ ID NO: 21 while maintaining functional equivalency thereto.

In addition, the [Z] may be the gene nucleic acid sequence of ORF2 to ORF7 regions of the BP2017-2 with accession number KCTC 13393BP, for example, the nucleic acid sequence of SEQ ID NO: 5, or a nucleic acid sequence having sequence identity of 70% or more, 75% or more, 80% or more, 85% or more, 90% or more, 95% or more, 96% or more, 97% or more, 98% or more, 99% or more, or 99.5% or more, within a range of maintaining equivalent functions to the sequence.

According to one embodiment of the present invention, [A] n may be further comprised at the 3′ end of the [Z] of Structural formula 1. The n is the number of nucleotides comprising base A, and it may be an integer of 10 to 100. Preferably, it may be an integer of 10 to 80, 10 to 70, 10 to 60, 10 to 50, 10 to 40, 10 to 30, 15 to 80, 15 to 70, 15 to 60, 15 to 50, 15 to 40, 15 to 30, 20 to 30, 20 to 26.

The polynucleotide may be DNA, RNA (when the nucleic acid sequence is represented on the basis of DNA, it is a sequence in which all or some of thymine (T) in the sequence is substituted with uracil (U)), reverse-transcriptome (DNA) of the RNA, or a combination thereof. The polynucleotide may have functions as genome of the chimeric virus of PRRSV.

Accordingly, other embodiment of the present invention provides a chimeric virus mutant strain of a Porcine Reproductive and Respiratory Syndrome Virus (PRRSV) comprising a polynucleotide having the structure of Structural formula 1. The chimeric virus mutant strain may comprise the polynucleotide having the structure of Structural formula 1 as genome.

In the present description, the term ‘chimeric virus mutant strain’ is to represent a virus modified by introducing (c) a gene encoding T cell epitope-EP7 derived from a porcine reproductive and respiratory syndrome virus into a chimeric virus comprising (a) the NSP1 gene (ORF1a and ORF1b) of the PRRS type II virus, for example, LMY parent strain (Accession No. GenBank accession no.DQ473474.1) or LMY ver2 virus with accession number 13394BP, and (b) ORF2 to ORF7 of the BP2017-2 with accession number KCTC 13393BP, and may also be represented by ‘chimeric virus comprising a gene encoding T cell epitope-EP7’, chimeric virus expressing EP7′, ‘PRRSV-EP7 chimeric virus’, or ‘chimeric virus of PRRSV’ in abbreviation.

In the present description, the term ‘chimeric virus mutant strain’ is to represent a virus modified by introducing (c) a gene encoding T cell epitope-EP8 derived from a porcine reproductive and respiratory syndrome virus into a chimeric virus comprising (a) the NSP1 gene (ORF1a and ORF1b) of the PRRS type II virus, for example, LMY parent strain (Accession No. GenBank accession no. DQ473474.1) or LMY ver2 virus with accession number 13394BP, and (b) ORF2 to ORF7 of the BP2017-2 with accession number KCTC 13393BP, and may also be represented by ‘chimeric virus comprising a gene encoding T cell epitope-EP8’, ‘chimeric virus expressing EP8’, ‘PRRSV-EP8 chimeric virus’, or ‘chimeric virus of PRRSV’ in abbreviation.

The genome of the chimeric virus mutant strain of PRRSV may be DNA or RNA, and preferably, it may be RNA.

The polynucleotide (genome of the chimeric virus mutant strain of PRRSV) having the structure of Structural formula 1 may be not natural, and may be recombinantly or synthetically prepared.

In one embodiment, the Structural formula 1 may comprise the nucleic acid sequence of SEQ ID NO: 6 or a nucleic acid sequence having sequence identity of 70% or more, 75% or more, 80% or more, 85% or more, 90% or more, 95% or more, 96% or more, 97% or more, 98% or more, 99% or more, or 99.5% or more within a range of maintaining equivalent functions to the nucleic acid sequence. For example, it may be the nucleic acid sequence of SEQ ID NO: 6.

In one embodiment, the Structural formula 1 may comprise the nucleic acid sequence of SEQ ID NO: 11 or a nucleic acid sequence having sequence identity of 70% or more, 75% or more, 80% or more, 85% or more, 90% or more, 95% or more, 96% or more, 97% or more, 98% or more, 99% or more, or 99.5% or more within a range of maintaining equivalent functions to the nucleic acid sequence. For example, it may be the nucleic acid sequence of SEQ ID NO: 11.

In Table 1 below, among the base sequence of the NSP1 gene of LMY ver2, the part in bold means the NSP1-beta region.

TABLE 1

| | | SEQ |
|---|---|---|
| Strain | | ID |
| origin | Base sequence (5′→3′) | NO |
| NSP1 of LMY | atgtctgggatacttgatcggtgcacgtgtacccccaatgccagggtgtttatggcggagggccaagtttac | 2 |
| ver2 | tgcacacgatgtctcagtgcacggtctctccttcccctgaatctccaagtttctgagctcggggtgctgggc | |
| | ctattctacaggcccgaagagccactccggtggacgttgccacgtgcattccccactgttgagtgctccccc | |
| | gccggcgcatgctggctttccgctatctttccaatcgcacgaatgaccagtggaaacctaaacttccaacaa | |
| | agaatggtacgggtcgccgccgaaatatacagagtcgggcaacttacgcccgctgtattgaaggctctgcaa | |
| | gtatacgagcggggttgtaggtggtatcccatcgtaggacctgtccctggagtggccgttttcgccaactcc | |
| | ctgcatgtgagtgatagacctttcccgggagcaactcacgtattaaccaacctgccactcccgcagagaccc | |
| | aagcctgaggacttttgccccttcgagtgtgctatggccgccgtatacgacgtcggacatggcgccgttatg | |
| | tacgttgccgacgagagagtctcctgggcccctcgcggcggcgacgaagttagattcgaaacggtcccacag | |
| | gagcttaagtcggttgcgaaccaattatgtacgtcgttcccaccccaccacgtagtcgatatgtctaagttc | |
| | gcctttaccgcccccggttgcggcgtatctatgcgggtcgaacgtcaatacggctgtctccccgccgatacg | |
| | gtccccgaaggcaactgttggtggagcttgttcgattcgctcccactcgaagtgcaaggcaaagagattcgc | |
| | cacgctaaccaattcgggtatcagactaagcatggcgtatccggtaagtacctacagcgtaggctgcaaatc | |
| | aacggtctccgcgcagtcgctgaccctaacggacctttcgtcgtacagtacttctccgtcaaggagagttgg | |
| | atccgccacttgaaactggccgaagaacctagttaccccgggttcgaggacctcctccgcataagggttgag | |
| | tctaatacgtcaccattggctaacaaggacgaaaaaattttccggtttggcagtcataagtggtacggc | |
| T cell | ctgctagctttcagtatcacatatactccagttatgatatacgcacttaaggtctctcgtgggcgtctccta | 4 |
| epitope-EP7 | gggctc | |
| T cell | ctgtggggcgtatattccgcaatcgaaacgtggaagttcataacgagtcgctgccgtctctgcttattaggg | 10 |
| epitope-EP8 | cgaaagtatatactcgctcctgctcaccacgtggagtcggct | |
| ORF2-ORF7 of | atgaaatggggtccatgcaaagccttttttacaaaattggccaactttttgtggatgctttcacggagttct | 5 |
| BP2017-2 | tggtgtccattgttgatatcattatatttttggccattttgtttggcttcaccatcgccggttggctggtgg | |
| | tcttttgcatcagattggtttgctccgcgatactccgtacgcgccctgccattcactctgagcaattacaga | |
| | agatcttatgaggcctttctttcccagtgccaagtggacattcccacctggggaactaaacatcctttgggg | |
| | atgctttggcaccataaggtgtcaaccctgattgatgaaatggtgtcgcgtcgaatgtaccgcatcatggaa | |
| | aaagcagggcaggctgcctggaaacaggtggtgagcgaggctacgctgtctcgcattagtagtttggatgtg | |
| | gtggctcattttcagcatctagccgccattgaagccgagacctgtaaatatttggcctcccggctgcccatg | |
| | ctacacaacctgcgcatgacaggttcaaatgtaaccatagtgtataatagcactttgaatcaggtgtttgct | |
| | atttttccaacccctggttcccggccaaagcttcatgattttcagcaatggttaatagctgtacattcctcc | |
| | atattttcctctgttgcagcttcttgtactctttttgttgtgctgtggttgcgggttccaatactacgtact | |
| | gtttttggtttccgctggttaggggcaatttttctttcgaactcacagtgaattacacggtgtgtccacctt | |
| | gcctcacccggcaagcagccacagagatctacgaacccggtaggtctctttggtgcaggatagggtatgacc | |
| | gatgtgaggaggatgatcatgacgagctagggtttatggtaccgcctggcctctccagcgaaggccacttga | |
| | ctagtgtttacgcctggttggcgttcttgtccttcagctacacggcccagttccatcccgagatattcggga | |
| | tagggaatgtgagtcgagtttatgttgacatcaaacatcaactcatctgcgccgaacatgacgggcagaaca | |
| | ccaccttgcctcgtcatgacaacatttcagccgtgtttcagacctattaccaacatcaagtcgacggcggca | |
| | attggtttcacctagaatggcttcgtcccttcttttcctcgtggttggttttaaatgtctcttggtttctca | |
| | ggcgttcgcctgcaaaccatgtttcagttcgagtcttgcagatattaagaccaacaccaccgcagcggcaag | |
| | ctttgctgtcctccaagacatcagttgccttaggcatcgcgactcggcctctgaggcgattcgcaaaatccc | |
| | tcagtgccgtacggcgatagggacacccgtgtatgttaccatcacagccaatgtgacagatgagaattattt | |
| | acattcttctgatctcctcatgctttcttcttgccttttctatgcttctgagatgagtgaaaagggatttaa | |
| | ggtggtatttggcaatgtgtcaggcatcgtggctgtgtgtgtcaattttaccagctacgtccaacatgtcaa | |
| | ggagtttacccaacgctccctggtggtcgaccatgtgcggttgctccatttcatgacacctgagaccatgag | |
| | gtgggcaactgttttagcctgtctttttgccattctgttggcaatttgaatgtttaagtatgttggagaaat | |
| | gcttgaccgcgggctgttgctcgcaattgctttctttgtggtgtatcgtgccgttctgttttgctgtgctcg | |
| | ccaacgccagcaacgacagcagctcccatctacagctgatttacaacttgacgctatgtgagctgaatggca | |
| | cagattggctagctaacaaatttgattgggcagtggagagttttgtcatctttcccgttttgactcacattg | |
| | tctcctatggtgccctcactaccagccatttccttgacacagtcgctttagtcactgtgtctaccgccgggt | |
| | ttgttcacgggcggtatgtcctaagtagcatctacgcggtctgtgccctggctgcgttgacttgcttcgtca | |
| | ttaggtttgcaaagaattgcatgtcctggcgctacgcgtgtaccagatataccaactttcttctggacacta | |
| | agggcggactctatcgttggcggtcgcctgtcatcatagagaaaaggggcaaagttgaggtcgaaggtcatc | |
| | tgatcgacctcaaaagagttgtgcttgatggttccgtggcaacccctataaccagagtttcagcggaacaat | |
| | ggggtcgtccttagatgacttctgtcatgatagcacggctccagaaaaggtgcttttggcgttttctattac | |
| | ctacacgccagtgatgatatatgccctaaaggtgagtcgcggccgactgctagggcttctgcaccttttgat | |
| | cttcctgaattgtgctttcaccttcgggtacatgactttcgcgcactttcagagtacaaataaggtcgcgct | |
| | cactatgggagcagtagttgcactcctttggggggtgtactcagccatagaaacctggaaattcatcacctc | |
| | cagatgccgtttgtgcttgctaggccgcaagtacattctggcccctgcccaccacgttgaaagtgccgcagg | |
| | ctttcatccgattgcggcaaatgataaccacgcatttgtcgtccggcgtcccggctccactacggtcaacgg | |
| | cacattggtgcccgggttaaaaagcctcgtgttgggtggcagaaaagctgttaaacagggagtggtaaacct | |
| | tgtcaaatatgccaaataacaacggcaagcagcagaagagaaagaagggggatggccagccagtcaatcagc | |
| | tgtgccagatgctgggtaagatcatcgctcagcaaaaccagtccagaggcaagggaccgggaaagaaaaata | |
| | agaagaaaaacccggagaagccccattttcctctagcgactgaagatgatgtcagacatcactttaccccta | |
| | gtgagcggcaattgtgtctgtcgtcaatccagaccgcctttaatcaaggcgctgggacttgcaccctgtcag | |
| | attcagggaggataagttacactgtggagtttagtttgcctacgcatcatactgtgcgcctgatccgcgtca | |
| | cagcatcaccctcagcatga | |
| [A]n | aaaaaaaaaaaaaaaaaaaaaaaa | 7 |
| Kozak | gccacc | 13 |
| TRS | gttccgtggcaacccctataaccagagtttcagcggaaca | 14 |
| (transcription | | |
| regulatory | | |
| sequence) 6 | | |
| Modified T | ggcgcgccgccaccatgctgctagctttcagtatcacatatactccagttatgatatacgcacttaaggtct | 20 |
| cell | ctcgtgggcgtctcctagggctctaggttccgtggcaacccctataaccagagtttcagcggaacaggcgcg | |
| epitope-EP7 | cc | |
| Modified T | ggcgcgccgccaccatgctgtggggcgtatattccgcaatcgaaacgtggaagttcataacgagtcgctgcc | 21 |
| cell | gtctctgcttattagggcgaaagtatatactcgctcctgctcaccacgtggagtcggcttaggttccgtggc | |
| epitope-EP8 | aacccctataaccagagtttcagcggaacaggcgcgcc | |

(In Table 1, the AscI restriction enzyme recognition site is underlined, and the Kozak sequence is in italics, and TRS6 is underlined and italicized, respectively, and the region between the Kozak sequence and TRS6 is the nucleic acid sequence encoding EP7 and/or EP8, and ATG (start codon) and TAG (stop codon) are attached and inserted back and forth of the EP7 and/or EP8 nucleic acid sequence)

TABLE 2

| | | SEQ |
|---|---|---|
| Strain | | ID |
| origin | Base Sequence (5′→3′) | NO |
| PRRSV-EP7 | ATGACGTATAGGTGTTGGCTCTATGCCTTGACATTTGTATTGTCAGGAGCTGTGACCATTGGCACAGTCCAA | 6 |
| chimera full | AACTTGCTGCGCAGAAACACCCTTCTGCGACAGCCTCCTTCAGGGGAGCTTGGGGTCTATCCCTAGCACCTT | |
| genome | GCTTCTGGAGTTGCACTGCTTTACGGTCTCTCCACCCCTTTAACCATGTCTGGGATACTTGATCGGTGCACG | |
| | TGTACCCCCAATGCCAGGGTGTTTATGGCGGAGGGCCAAGTTTACTGCACACGATGTCTCAGTGCACGGTCT | |
| | CTCCTTCCCCTGAATCTCCAAGTTTCTGAGCTCGGGGTGCTGGGCCTATTCTACAGGCCCGAAGAGCCACTC | |
| | CGGTGGACGTTGCCACGTGCATTCCCCACTGTTGAGTGCTCCCCCGCCGGCGCATGCTGGCTTTCCGCTATC | |
| | TTTCCAATCGCACGAATGACCAGTGGAAACCTAAACTTCCAACAAAGAATGGTACGGGTCGCCGCCGAAATA | |
| | TACAGAGTCGGGCAACTTACGCCCGCTGTATTGAAGGCTCTGCAAGTATACGAGCGGGGTTGTAGGTGGTAT | |
| | CCCATCGTAGGACCTGTCCCTGGAGTGGCCGTTTTCGCCAACTCCCTGCATGTGAGTGATAGACCTTTCCCG | |
| | GGAGCAACTCACGTATTAACCAACCTGCCACTCCCGCAGAGACCCAAGCCTGAGGACTTTTGCCCCTTCGAG | |
| | TGTGCTATGGCCGCCGTATACGACGTCGGACATGGCGCCGTTATGTACGTTGCCGACGAGAGAGTCTCCTGG | |
| | GCCCCTCGCGGCGGCGACGAAGTTAGATTCGAAACGGTCCCACAGGAGCTTAAGTCGGTTGCGAACCAATTA | |
| | TGTACGTCGTTCCCACCCCACCACGTAGTCGATATGTCTAAGTTCGCCTTTACCGCCCCCGGTTGCGGCGTA | |
| | TCTATGCGGGTCGAACGTCAATACGGCTGTCTCCCCGCCGATACGGTCCCCGAAGGCAACTGTTGGTGGAGC | |
| | TTGTTCGATTCGCTCCCACTCGAAGTGCAAGGCAAAGAGATTCGCCACGCTAACCAATTCGGGTATCAGACT | |
| | AAGCATGGCGTATCCGGTAAGTACCTACAGCGTAGGCTGCAAATCAACGGTCTCCGCGCAGTCGCTGACCCT | |
| | AACGGACCTTTCGTCGTACAGTACTTCTCCGTCAAGGAGAGTTGGATCCGCCACTTGAAACTGGCCGAAGAA | |
| | CCTAGTTACCCCGGGTTCGAGGACCTCCTCCGCATAAGGGTTGAGTCTAATACGTCACCATTGGCTAACAAG | |
| | GACGAAAAAATTTTCCGGTTTGGCAGTCATAAGTGGTACGGCGCTGGTAAGAGAGCAAGGAAAGCACGCTCT | |
| | CGTGCGACTAATACAGTCGCTGACCGCGCTTTGTCCGTTCGTGAAATCTGGCAGGCCAAGGAGCATGAGGTT | |
| | ACCGGCGCCAATAAGGCTGAGCACCTCAAACACTACTCCCCACCTGCCGAAGGGAATTGTGGTTGGCACTGC | |
| | ATTTCCGCCATCGTCAACCGGATGGTTAATTCCAAATTTGAAACCACCCTTCCCGAAAGAGTGAGACCTCCA | |
| | GATGACTGGGCTACCGACGAGGATCTTGCGAATGCCATCCAAATCCTCAGACTCCCTGCGGCCTTAGACAGG | |
| | AACGGTGCTTGTGCTAGCGCCAAGTACGTACTTAAGCTGGAAGGTGAGCATTGGACTGTCACTGTGACCCCT | |
| | GGGATGTCCCCTTCTTTGCTCCCTCTTGAATGTGTTCAGGGCTGTTGTGGGCACAAGGGCGGTCTTGGTTCC | |
| | CCAGACGCAGTCGAGGTCTCCGGATTTGACCCTGCCTGCCTTGACCGTCTGGCTGAGGTGATGCACCTGCCT | |
| | AGCAGTGCTATCCCAGCCGCTCTGGCCGAAATGTCTGGTGATTCCGATCGTTCGGCTTCTCCGGTCACCACC | |
| | GTGTGGACTGTTTCGCAGTTCTTTGCCCGTCACAGCGGAGGAAATCACCCTGACCAAGTGCGCTTAGGGAAA | |
| | ATCATCAGCCTTTGTCAGGTGATTGAGGACTGCTGCTGTTCCCAGAACAAAACCAACCGGGTCACCCCGGAG | |
| | GAGGTTGCAGCAAAGATTGACCTGTACCTCCGCGGTGCAACAAATCTTGAAGAATGCTTGGCCAGGCTCGAG | |
| | AAAGCGCGCCCGCCACGGGTAATCGACACCTTCTTTGATTGGGACGTTGTGCTCCCTGGGGTTGAGGCAGCA | |
| | ACCCAAACGACCAAGCTGCCCCAGGTCAACCAGTGTCGTGCTCTGGTCCCTGTTGTGACTCAAAAGTCCTTG | |
| | GACAACAACTCGGTCCCCCTGACCGCCTTTTCACTGGCTAACTACTACTACCGCGCGCAAGGTGACGAAGTT | |
| | TGTCACCGTGAAAGACTTAATGCCGTGCTTTCCAAGCTGGAAGAGGTTGTCCGAGAAGAGTACGGGCTCATG | |
| | CCTACCGGGCCTGGCCCACGGCCCACATTGCCACGCGGGCTCGACGAACTCAAAGACCAGATGGAGGTGGAC | |
| | TTGCTGAAACTGGCTAACGCCCAGGCGACTTCTGACATGATGGCTTGGGCAGTCGAGCAGGTTGACCTAAAA | |
| | ACTTGGGTCAAGAACTACCCGCGGTGGATGCCACCACCTCCTCCGCCAAAAGTTCGGCCTCGAAAAACGAAG | |
| | CCTGTCAAGAGCTTGCCAGAGGGAAAGCCCGCCCCCGCCCCTCGCAGGAGGGTTGGGACTGGTTGTGGCAGC | |
| | CCGACTTCATTGGGCGATGATGTTCTTAACAGTTGGGAAGATTTGGCTGTTGGCAGCCCCTCGGGTCTCCCG | |
| | ACCCCCCCTGAGCCGGCAACACCTTCGAGTGAGCCAGTGGTTGTGTCAGCACCGCAGTGCATCTTCAGACCG | |
| | GCGACGCCCTTGAGTGAGCCGACCCCAATTCCCGCACCCCGTGGGACTGCGTCTCGACCAGTGACACCTTTG | |
| | AGCGAGCCGATCCCTGTGCCTGCACCGCGGCGTAAATTTCAGCAGGCGAGAAAGTTGAGTTCGGCGGTGGTA | |
| | ATTCCGCCGTACCAGGACGAGCCCCTGGATTTGTCCGCTTCCTCGCTGACTGAATATGAAGCCCTTCCTCTA | |
| | GCGCCGCCGCAAAGCGAGGGCGCTCTAAGAGTGGAGGGGCATGAAGTTGAGGAAGCCCTGAGTGAAATCTCA | |
| | GACATGTCGGGTGGCACTAAACCTGCGCCCGTATCATCAAGCAGCTCCTTGTCCAGCGTGAGAATCACACGC | |
| | CCAAAACACTCAGCTCAAGCCATCATCGACTCGGTCGGGCCCTGCAGTGGGCATCTCCAAGAGGTGAAGGAG | |
| | ATATGCCTTAGTGTCATGCGCGAGGCATGTGATGCGACTAAGCTCAATGACCCCGCTACGCAGGAATGGCTC | |
| | TCTCGCATGTGGGATCGGGTGGACATGCTGACTTGGCGCAACACGTCTGCTTACCAGACGCTTAGCACCTTA | |
| | GGTGGCTGGTCAAGGTTCCTCCCAAAATTGATACTTGAGACACCGCCGCCCTACCCGTGTGAGTTTGTGATG | |
| | ATGCCTTACACGCCTGCACCTTCCGTGGGTGCGGAGAGCGACCTCACCATTGGCTCAGTTGCTACTGAGGAT | |
| | GTTCCACGCATCCTTGAGAACATGGAGAATGTCGGCGAGATGACTAACCAGGGACCCTTGGCCTTCTCCGAG | |
| | GATAAACCGGTGGATGACCAACCTTCCGAAGACCTCCGGACATCGTCGCAAGGACCTGACGGGAGCACATCA | |
| | GCTCCGTCCGCAGATACAGCTGGCGCCGGCTCATCCACCGATTTGCCGTCTTCAGGCGGCGTGGATGCGGAC | |
| | GGAGGGGGACCGCTTCGGGCGGTAAAAAGAAAAACCGAACGGCTCTTTGACCAACTGAGTCGTCAGGTTTTT | |
| | GACCTCGTCTCCCATCTCCCTATTTTCTTCTCACGCCTCTTCAACCCTGGCGGTGGTTACTCTCCGGGTGAT | |
| | TGGGGTTTTGCAGCTTTTACTCTATTGTGCCTCTTTTTATGTTACAGTTACCCAGCATTTGGCATCGTTCCC | |
| | CTCTTGGGTGTATTTTCTGGGTCTTCTCGGCGCGTGCGAATGGGGGTTTTTGGCTGCTGGTTGGCTTTTGCT | |
| | GTTGGTCTGTTCAAGCCTGTGTCCGACCCAGTCGGCGCTGCTTGTGAGTTTGACTCGCCAGAATGCAGAAAC | |
| | ATCCTTCATTCTTTTGAGCTTCTCAAACCTTGGGACCCTGTTCGCAGCCTTGTTGTGGGCCCCGTCGGTCTC | |
| | GGTTTTGCCATTCTTGGCAGGCTACTGGGCGGGGCACGCAGCATCTGGCACTTTTTGCTTAGGCTTGGCATT | |
| | GTTGCAGACTGTGTCTTGGCTGGAGCTTATGTGCTTTCTCAAGGTAGGTGCAAAAAGTGTTGGGGATCTTGT | |
| | ATAAGAACCGCTCCTAATGAGGTCGCTTTCAACGTGTTTCCTTTTACGCGTGCAACCAGGTCGTCACTTGTT | |
| | GACCTTTGCGATCGGTTTTGTGCGCCGAGAGGCATGGACCCCATTTTTCTCGCCACTGGGTGGCGTGGATGC | |
| | TGGGCCGGTCGGAGCCCCATTGAGCAACCCTCTGAAAAACCCATCGCGTTTGCCCAATTGGATGAAAAGAAG | |
| | ATTACGGCTAGGACTGTGGTCGCCCAGCCTTATGATCCCAACCAAGCCGTAAAGTGCCTGCGGGTATTGCAG | |
| | GCGGGAGGGGCGATGGTGGCTGAGGCGGTCCCAAAAGTGGTCAGGGTTTCCGCTGTTCCATTTCGAGCCCCC | |
| | TTTTTTCCTACCGGAGTGAAGGTTGACCCTGAATGTAGAGTAGTGGTTGACCCTGACACTTTCACTGCAGCT | |
| | CTCCGGTCTGGTTACTCCACCACAAACCTCGTCCTCGGTGTGGGGGACTTTGCCCAGCTGAATGGATTAAAA | |
| | ATCAGGCAAATTTCTAAGCCTTCAGGGGGGGGCCCACATCTCATGGCTGCCCTGCATGTTGCCTGCTCGATG | |
| | ATTTTGCACATGTTTGCTGGGATTTATGTGACTGCGGTGGGTTCTTGCGGCACCGGCACCAACGATCCGTGG | |
| | TGCGCAAACCCATTTGCCGTCCCTGGCTACGGACCTGGCTCCCTCTGCACGTCCAGATTGTGTATTTCCCAG | |
| | CACGGCCTTACCCTGCCCTTGACAGCACTTGTGGCGGGATTCGGCATCCAGGAAATTGCCTTAGTCGCTTTG | |
| | ATCTTTGTTTCCATCGGAGGCATTGCTCATAGGTTGAGTTGTAAAGCTGACATGCTGTGTGTTTTGCTTGCA | |
| | ATTGCCAGCTATGTTTGGGTACCTCTTACCTGGTTACTTTGTGTTTTTCCTTGCTGGTTGCGCTGGTTTTCT | |
| | TTGCATCCCCTTACCGTCCTATGGTTGGTGTTTTTCTTGATTTCTGTGAATATGCCTTCAGGAATCTTGGCC | |
| | ATGGTGTTGTTAGTTTCTCTTTGGCTTCTTGGTCGTTATACTAATGTCGCCGGTCTTGTCACTCCCTACGAC | |
| | ATTCATCATTACACCAGTGGCCCCCGCGGTGTTGCCGCATTGGCTACTGCACCAGATGGAACCTACTTAGCC | |
| | GCTGTTCGCCGCGCTGCGTTGACTGGCCGCACCATGTTGTTCACCCCGTCCCAGCTTGGGTCTCTTCTTGAG | |
| | GGCGCTTTCAGAACTCGAAAGCCCTCACTGAACACCGTCAATGTGGTCGGGTCCTCCATGGGCTCTGGCGGG | |
| | GTGTTTACCATTGACGGGAAAGTCAGGTGCGTGACTGCCACACATGTCCTTACGGGTAACTCAGCCAGGGTT | |
| | TCCGGAACCGGCTTCAATCAAATGCTTGACTTTGACGTAAAAGGGGATTTCGCTATAGCCGATTGCCCGAAT | |
| | TGGCAAGGGGCTGCCCCCAAAACCCAATTCTGCGAGGATGGATGGACTGGCCGTGCCTATTGGCTAACATCC | |
| | TCTGGCGTCGAACCCGGCGTCATTGGGAAAGGATTCGCCTTCTGCTTCACCGCGTGCGGCGATTCCGGGTCC | |
| | CCAGTGATCACCGAGGCCGGTGAGCTTGTCGGTGTTCACACGGGATCGAATAAACAAGGGGGGGGTATTGTC | |
| | ACACGCCCCTCAGGCCAGTTTTGTAATGTGGCACCCATCAAGCTGAGCGAATTAAGTGAATTCTTTGCTGGG | |
| | CCTAAGGTCCCGCTCGGTGATGTGAAGGTTGGCAGCCACATAATCAAAGACACAAGCGAGGTGCCTTCAGAT | |
| | CTTTGTGCCTTGCTTGCTGCCAAACCTGAACTGGAAGGAGGTCTCTCCACCGTCCAACTTCTGTGTGTGTTT | |
| | TTCCTTCTGTGGAGAATGATGGGACATGCCTGGACGCCCTTGGTTGCTGTGGGTTTCTTTATTTTGAATGAG | |
| | ATTCTCCCGGCCGTCCTGGTCCGGAGTATTTTCTCCTTTGGAATGTTTGTGCTATCCTGGCTCACGCCATGG | |
| | TCTGCGCAAGTTCTGATGATCAGGCTCCTGACAGCAGCTCTTAACAGGAATAGATGTTCACTTGCCTTTTTC | |
| | AGCCTCGGTGCAGTGACCGGTTTTGTTGCAGATCTTGCGGCTACTCAGGGGCATCCGTTGCAGGCAGTGATG | |
| | AATTTGAGCACCTATGCATTCCTGCCTCGGGTAATGGTTGTGACTTCACCAGTCCCAGTGATCGCGTGTGGT | |
| | GTTGTGCACCTACTCGCCATCATTCTGTACCTGTTTAAATACCGTAGCCTGCACCATATCCTTGTTGGCGAT | |
| | GGAGTGTTCTCTTCGGCCTTCTTCCTGCGATATTTTGCCGAAGGAAAGTTGAGGGAAGGGGTGTCACAATCC | |
| | TGCGGAATGAATCATGAGTCTCTGACTGGTGCCCTCGCTATGAGACTCAATGACGAGGACTTGGATTTCCTC | |
| | ATGAAATGGACTGATTTTAAGTGCTTCGTTTCTGCGTCCAACATGAGGAATGCAGCGGGTCAATTTATCGAG | |
| | GCTGCATATGCTAAAGCACTTAGAGTTGAACTGGCCCAGTTGGTGCAGGTTGATAAGGTTCGAGGTACTATG | |
| | GCCAAACTTGAAGCTTTTGCTGATACTGTGGCACCTCAACTCTCGCCCGGTGATATTGTTGTCGCTCTCGGC | |
| | CACACGCCTGTTGGCAGTATCTTCGACTTAAAGGTTGGTAGCACCAAGCATACCCTCCAAGCCATTGAGACC | |
| | AGGGTTCTTGCTGGGTCCAAAATGACCGTGGCGCGCGTCGTCGACCCGACCCCCACGCCCCCACCCGCGCCC | |
| | GTGCCCATCCCCCTCCCACCGAAAGTTCTGGAGAATGGCCCCAACGCTTGGGGGGATGAGGACCGTTTGAAT | |
| | AAGAAGAAAAGACGCAGGATGGAAGCCCTCGGCATCTATGTTATGGGCGGGAAAAAGTACCAGAAATTTTGG | |
| | GACAAGAACTCCGGTGATGTGTTTTATGAGGAGGTCCATAACAACACAGATGAGTGGGAGTGTCTCACAGTT | |
| | GGCGACCCTGCCGACTTTGACCCTGAGAAGGGAACCCTGTGTGGACATGTCACCATTGACAACAAGGCTTAC | |
| | CATGTTTACACCTCCTCATCTGGTAAGAAGTTCTTGGTTCCCGTCAACCCAGAGAACGGAAGAGTTCAATGG | |
| | GAAGCTGCAAAGCTTTCCGTGGAGCAGGCCCTAGGCATGATGAATGTCGACGGCGAACTGACTGCCAAGGAA | |
| | CTGGAGAAACTGAAAAGAATAATTGACAAACTCCAGGGCCTGACTAAGGAGCAGTGTTTAAACTGCTAGCCG | |
| | CCAGCGACTTGACCCGCTGTGGTCGCGGCGGCTTGGTTGTTACTGAGACAGCGGTGAAAATAGTCAAATTTC | |
| | ACAATCGGACTTTCACTCTGGGGCCTGTGAATTTAAAAGTGGCCAGTGAGGTTGAGCTAAAAGATGCGGTTG | |
| | AGCACAACCAACACCCGGTTGCGAGGCCGATCGATGGTGGAGTTGTGCTCCTGCGTTCCGCGGTTCCCTCGC | |
| | TTATAGACGTCTTGATCTCCGGTGCTGACACATCTCCCAAGTTACTTGCCCTTCACGGGCCGGGAAACACTG | |
| | GGATCGATGGCACGCTCTGGGATTTCGAGTCCGAAGCCACTAAAGAGGAAGTCGCACTCAGTGCGCAAATAA | |
| | TACAGGCTTGTGACATTAGGCGTGGCGACGCTCCTGAAATTGGTCTCCCTTATAAGCTGTACCCTGTTAGGG | |
| | GCAACCCTGAGCGGGTGAAAGGAGTTCTGCAGAATACAAGGTTTGGAGACATACCCTACAAAACCCCTAGTG | |
| | ACACCGGGAGCCCAGTGCACGCGGCTGCCTGCCTTACGCCCAACGCCACTCCTGTGACTGATGGGCGTTCCG | |
| | TCTTGGCTACAACCATGCCCCCCGGGTTTGAGTTATATGTACCGACCATACCAGCGTCTGTCCTTGATTACC | |
| | TTGACTCTAGGCCTGACTGCCCTAAACAGCTGACAGAGCACGGCTGCGAAGACGCCGCACTGAAAGACCTCT | |
| | CTAAATATGACTTATCTACCCAAGGCTTTGTCTTACCTGGGGTTCTTCGCCTTGTGCGGAAATACCTGTTTG | |
| | CCCATGTAGGTAAATGTCCACCCGTTCATCGGCCTTCTATTTACCCCGCTAAGAATTCTATGGCTGGAATAA | |
| | ATGGGAACAGGTTCCCAACCAAAGACATCCAGAGCGTTCCTGAAATCGACGTTCTGTGCGCACAGGCTGTGC | |
| | GGGAAAACTGGCAAACTGTCACCCCTTGTACTCTTAAGAAACAGTATTGCGGGAAGAAGAAGACCAGGACCA | |
| | TACTCGGCACCAATAACTTCATTGCACTGGCCCACCGAGCAGTGTTGAGTGGTGTCACCCAGGGCTTCATGA | |
| | AAAAGGCGTTCAACTCGCCCATCGCCCTCGGAAAGAACAAGTTCAAGGAGCTACAGACTCCGGTCCTGGGCA | |
| | GGTGCCTTGAAGCTGACCTCGCATCCTGTGATCGATCCACGCCTGCAATTGTTCGCTGGTTTGCTGCCAACC | |
| | TTCTTTATGAACTTGCCTGTGCTGAAGAGTATCTACCGTCGTATGTGCTGAACTGCTGCCACGACTTACTAG | |
| | TCACGCAGTCCGGCGCAGTGACTAAGAGAGGTGGCCTGTCGTCTGGCGACCCGATCACCTCTGTGTCTAACA | |
| | CCATTTACAGTTTGGTGATCTATGCACAGCACATGGTGCTTAGTTACTTTAAAAGTGGTCACCCCCATGGCC | |
| | TTCTGTTCTTACAAGACCAGCTAAAGTTTGAGGACATGCTCAAGGTTCAACCCCTGATCGTCTATTCGGACG | |
| | ATCTCGTGCTGTATGCCGAGTCTCCCACCATGCCAAATTATCACTGGTGGGTTGAGCATCTGAATTTGATGC | |
| | TGGGGTTTCAGACGGACCCAAAAAAAACAGCAATAACAGATTCGCCATCATTTCTGGGCTGTAGAATAATGA | |
| | ATGGGCGCCAGCTAGTCCCCAACCGTGACAGGATCCTCGCGGCCCTCGCCTATCACATGAAGGCAAGTAATG | |
| | TTTCTGAATACTATGCCTCAGCGGCTGCAATACTCATGGACAGCTGTGCTTGTTTGGAGTATGATCCTGAAT | |
| | GGTTTGAAGAACTTGTAGTTGGAATAGCGCAGTGCGCCCGCAAGGATGGCTACAGCTTTCCCGGCACGCCGT | |
| | TCTTCATGTCCATGTGGGAAAAACTCAGGTCCAATTATGAGGGGAAGAAGTCGAGAGTGTGCGGGTACTGCG | |
| | GGGCCCCGGCCCCGTACGCTACTGCCTGTGGCCTCGACGTCTGCATCTACCACACCCACTTCCACCAGCATT | |
| | GTCCAGTCACAATCTGGTGTGGCCATCCAGCGGGTTCTGGTTCTTGTAGTGAGTGCAAATCCCCTGTAGGGA | |
| | AAGGCACAAACCCTTTAGACGAGGTGCTGGAACAAGTCCCGTATAAGCCCCCACGGACCGTTATCATGCATG | |
| | TAGAGCAGGGTCTCACCCCCCTTGATCCAGGTAGATACCAAACTCGCCGCGGATTAGTTTCCGTTAGGCGTG | |
| | GAATTAGGGGAAATGAAGTTGAACTACCAGACGGTGATTATGCTAGCACCGCCTTGCTCCCTACCTGCAAAG | |
| | AGATCAACATGGTCGCTGTCGCTTCCAATGTATTGCGCAGCAGGTTCATCATCGGCCCACCCGGTGCAGGGA | |
| | AAACATACTGGCTCCTTCAACAGGTCCAGGACGGTGATGTTATTTACACACCAACTCACCAGACCATGCTTG | |
| | ACATGATCAGGGCTTTGGGGACGTGCCGGTTCAACGTCCCGGCAGGCACAACGCTGCAATTCCCTGTCCCCT | |
| | CCCGCACCGGTCCGTGGGTTCGCATCCTGGCCGGCGGTTGGTGTCCTGGCAAGAATTCCTTCCTAGATGAAG | |
| | CAGCGTATTGCAACCACCTTGATGTTTTGAGGCTTCTTAGCAAAACTACCCTCACCTGTCTAGGAGACTTCA | |
| | AGCAACTCCACCCAGTGGGTTTTGATTCTCATTGTTATGTTTTTGACATCATGCCTCAAACTCAACTGAAGA | |
| | CCATCTGGAGGTTCGGACAGAATATCTGTGATGCCATCCAGCCAGATTACAGGGACAAGCTCATGTCCATGG | |
| | TCAACACAACCCGTGTGACCTACGTGGAAAGACCTGTCAGGTATGGGCAGGTCCTCACCCCCTACCACAGGG | |
| | ACCGAGAGGACGACGCCATCACTATTGACTCCAGTCAAGGCGCCACATTCGATGTGGTCACATTGCATTTGC | |
| | CCACTAAAGATTCACTCAACAGGCAAAGAGCCCTTGTTGCCATCACCAGGGCTAGACACGCTATCTTTGTGT | |
| | ATGACCCACACAGGCAGCTGCAGAGCTTGTTTGATCTTCCTGCAAAAGGCACACCCGTCAACCTTGCAGTGC | |
| | ACCGCGACGGGCAGCTGATCGTACTTGATAGAAATAACAAAGAATGCACGGTTGCTCAGGCTCTAGGCAACG | |
| | GGGATAAATTCAGGGCCACAGACAAGCGTGTTGCAGATTCACTCCGCGCCATTTGTGCTGATCTAGAAGGGT | |
| | CGAGTTCTCCGCTCCCCAAGGTCGCACACAACTTGGGGTTTTATTTCTCACCTGACTTAACACAATTTGCCA | |
| | AACTCCCAGTGGAACTTGCACCTCATTGGCCCGTGGTGACAACCCAGAACAATGAAAAGTGGCCAGATCGGC | |
| | TGGTCGCCAGCCTTCGCCCTATCCATAAATACAGCCGCGCGTGCATCGGTGCCGGCTATATGGTGGGCCCTT | |
| | CGGTGTTTCTAGGCACTCCTGGGGTCGTATCATACTTTCTTACAAAATTTGTTAAAGGCGAGGCTCAAATGC | |
| | TTCCAGAAACAGTCTTCAGCACCGGCCGAATTGAGGTGGATTGCCGGGAATATCTTGACGATCGGGAGCGGG | |
| | AAGTTGCTGCATCCCTCCCACACGCCTTTATTGGCGACGTCAAAGGCACCACCGTTGGCGGATGTCATCATG | |
| | TCACCTCCAGGTACCTTCCTCGCTTCCTTCCCAAAGAGTCAGTTGCGGTAGTCGGGGTCTCAAGCCCCGGGA | |
| | AAGCCGCGAAGGCAATGTGCACACTGACAGATGTGTACCTCCCAGATCTTGAAGCCTATCTCCACCCGGAGA | |
| | CCCAGTCCAGGTGCTGGAAAATGATGTTGGACTTCAAAGAAGTTCGACTGATGGTCTGGAAAGACAAAACGG | |
| | CCTATTTCCAACTTGAGGGTCGTTATTTCACCTGGTATCAGCTCGCTAGTTATGCCTCGTACGTCCGTGTTC | |
| | CTGTCAACTCCACGGTGTACTTGGACCCCTGCATGGGCCCAGCCCATTGCAACAGACGAGTTGTCGGGTCCA | |
| | CTCAGTGGGGAGCTGACCTCGCAATCACCCCCTATGATTATGGCGCCAAGATCATTTTGTCTAGCGCATATC | |
| | ACGGTGAAATGCCCCCTGGGTACAAGATTCTGGCGTGTGCGGAGTTCTCATTGGATGACCCGGTCAGATACA | |
| | AACACACCTGGGGATTTGAGTCGGATACAGCGTACCTGTACGAGTTCACTGGAAATGGTGAGGATTGGGAAG | |
| | ATTATAATGATGCGTTTCGTGCGCGCCAGAAAGGGAAAATTTATAAGGCCACTGCCACCAGCTTGAAGTTTT | |
| | ATTTCCCTCCGGGCCCTGTCGTTGAACCAACTTTGGGCCTGAACTGAAGGCGCGCCGCCACCATGCTGCTAG | |
| | CTTTCAGTATCACATATACTCCAGTTATGATATACGCACTTAAGGTCTCTCGTGGGCGTCTCCTAGGGCTCT | |
| | AGGTTCCGTGGCAACCCCTATAACCAGAGTTTCAGCGGAACAGGCGCGCCATGAAATGGGGTCCATGCAAAG | |
| | CCTTTTTTACAAAATTGGCCAACTTTTTGTGGATGCTTTCACGGAGTTCTTGGTGTCCATTGTTGATATCAT | |
| | TATATTTTTGGCCATTTTGTTTGGCTTCACCATCGCCGGTTGGCTGGTGGTCTTTTGCATCAGATTGGTTTG | |
| | CTCCGCGATACTCCGTACGCGCCCTGCCATTCACTCTGAGCAATTACAGAAGATCTTATGAGGCCTTTCTTT | |
| | CCCAGTGCCAAGTGGACATTCCCACCTGGGGAACTAAACATCCTTTGGGGATGCTTTGGCACCATAAGGTGT | |
| | CAACCCTGATTGATGAAATGGTGTCGCGTCGAATGTACCGCATCATGGAAAAAGCAGGGCAGGCTGCCTGGA | |
| | AACAGGTGGTGAGCGAGGCTACGCTGTCTCGCATTAGTAGTTTGGATGTGGTGGCTCATTTTCAGCATCTAG | |
| | CCGCCATTGAAGCCGAGACCTGTAAATATTTGGCCTCCCGGCTGCCCATGCTACACAACCTGCGCATGACAG | |
| | GTTCAAATGTAACCATAGTGTATAATAGCACTTTGAATCAGGTGTTTGCTATTTTTCCAACCCCTGGTTCCC | |
| | GGCCAAAGCTTCATGATTTTCAGCAATGGTTAATAGCTGTACATTCCTCCATATTTTCCTCTGTTGCAGCTT | |
| | CTTGTACTCTTTTTGTTGTGCTGTGGTTGCGGGTTCCAATACTACGTACTGTTTTTGGTTTCCGCTGGTTAG | |
| | GGGCAATTTTTCTTTCGAACTCACAGTGAATTACACGGTGTGTCCACCTTGCCTCACCCGGCAAGCAGCCAC | |
| | AGAGATCTACGAACCCGGTAGGTCTCTTTGGTGCAGGATAGGGTATGACCGATGTGAGGAGGATGATCATGA | |
| | CGAGCTAGGGTTTATGGTACCGCCTGGCCTCTCCAGCGAAGGCCACTTGACTAGTGTTTACGCCTGGTTGGC | |
| | GTTCTTGTCCTTCAGCTACACGGCCCAGTTCCATCCCGAGATATTCGGGATAGGGAATGTGAGTCGAGTTTA | |
| | TGTTGACATCAAACATCAACTCATCTGCGCCGAACATGACGGGCAGAACACCACCTTGCCTCGTCATGACAA | |
| | CATTTCAGCCGTGTTTCAGACCTATTACCAACATCAAGTCGACGGCGGCAATTGGTTTCACCTAGAATGGCT | |
| | TCGTCCCTTCTTTTCCTCGTGGTTGGTTTTAAATGTCTCTTGGTTTCTCAGGCGTTCGCCTGCAAACCATGT | |
| | TTCAGTTCGAGTCTTGCAGATATTAAGACCAACACCACCGCAGCGGCAAGCTTTGCTGTCCTCCAAGACATC | |
| | AGTTGCCTTAGGCATCGCGACTCGGCCTCTGAGGCGATTCGCAAAATCCCTCAGTGCCGTACGGCGATAGGG | |
| | ACACCCGTGTATGTTACCATCACAGCCAATGTGACAGATGAGAATTATTTACATTCTTCTGATCTCCTCATG | |
| | CTTTCTTCTTGCCTTTTCTATGCTTCTGAGATGAGTGAAAAGGGATTTAAGGTGGTATTTGGCAATGTGTCA | |
| | GGCATCGTGGCTGTGTGTGTCAATTTTACCAGCTACGTCCAACATGTCAAGGAGTTTACCCAACGCTCCCTG | |
| | GTGGTCGACCATGTGCGGTTGCTCCATTTCATGACACCTGAGACCATGAGGTGGGCAACTGTTTTAGCCTGT | |
| | CTTTTTGCCATTCTGTTGGCAATTTGAATGTTTAAGTATGTTGGAGAAATGCTTGACCGCGGGCTGTTGCTC | |
| | GCAATTGCTTTCTTTGTGGTGTATCGTGCCGTTCTGTTTTGCTGTGCTCGCCAACGCCAGCAACGACAGCAG | |
| | CTCCCATCTACAGCTGATTTACAACTTGACGCTATGTGAGCTGAATGGCACAGATTGGCTAGCTAACAAATT | |
| | TGATTGGGCAGTGGAGAGTTTTGTCATCTTTCCCGTTTTGACTCACATTGTCTCCTATGGTGCCCTCACTAC | |
| | CAGCCATTTCCTTGACACAGTCGCTTTAGTCACTGTGTCTACCGCCGGGTTTGTTCACGGGCGGTATGTCCT | |
| | AAGTAGCATCTACGCGGTCTGTGCCCTGGCTGCGTTGACTTGCTTCGTCATTAGGTTTGCAAAGAATTGCAT | |
| | GTCCTGGCGCTACGCGTGTACCAGATATACCAACTTTCTTCTGGACACTAAGGGCGGACTCTATCGTTGGCG | |
| | GTCGCCTGTCATCATAGAGAAAAGGGGCAAAGTTGAGGTCGAAGGTCATCTGATCGACCTCAAAAGAGTTGT | |
| | GCTTGATGGTTCCGTGGCAACCCCTATAACCAGAGTTTCAGCGGAACAATGGGGTCGTCCTTAGATGACTTC | |
| | TGTCATGATAGCACGGCTCCAGAAAAGGTGCTTTTGGCGTTTTCTATTACCTACACGCCAGTGATGATATAT | |
| | GCCCTAAAGGTGAGTCGCGGCCGACTGCTAGGGCTTCTGCACCTTTTGATCTTCCTGAATTGTGCTTTCACC | |
| | TTCGGGTACATGACTTTCGCGCACTTTCAGAGTACAAATAAGGTCGCGCTCACTATGGGAGCAGTAGTTGCA | |
| | CTCCTTTGGGGGGTGTACTCAGCCATAGAAACCTGGAAATTCATCACCTCCAGATGCCGTTTGTGCTTGCTA | |
| | GGCCGCAAGTACATTCTGGCCCCTGCCCACCACGTTGAAAGTGCCGCAGGCTTTCATCCGATTGCGGCAAAT | |
| | GATAACCACGCATTTGTCGTCCGGCGTCCCGGCTCCACTACGGTCAACGGCACATTGGTGCCCGGGTTAAAA | |
| | AGCCTCGTGTTGGGTGGCAGAAAAGCTGTTAAACAGGGAGTGGTAAACCTTGTCAAATATGCCAAATAACAA | |
| | CGGCAAGCAGCAGAAGAGAAAGAAGGGGGATGGCCAGCCAGTCAATCAGCTGTGCCAGATGCTGGGTAAGAT | |
| | CATCGCTCAGCAAAACCAGTCCAGAGGCAAGGGACCGGGAAAGAAAAATAAGAAGAAAAACCCGGAGAAGCC | |
| | CCATTTTCCTCTAGCGACTGAAGATGATGTCAGACATCACTTTACCCCTAGTGAGCGGCAATTGTGTCTGTC | |
| | GTCAATCCAGACCGCCTTTAATCAAGGCGCTGGGACTTGCACCCTGTCAGATTCAGGGAGGATAAGTTACAC | |
| | TGTGGAGTTTAGTTTGCCTACGCATCATACTGTGCGCCTGATCCGCGTCACAGCATCACCCTCAGCATGATG | |
| | GGCTGGCATTCTTGAGGCATCTCAGTGTTTGAATTGGAAGAATGTGTGGTGAATGGCACTGATTGACATTGT | |
| | GCCTCTAAGTCACCTATTCAATTAGGGCGACCGTGTGGGGGTGAGATTTAATTGGCGAGAACCATGCGGCCG | |
| | AAATTAAAAAAAAAAAAAAAAAAAAAAAA | |
| PRRSV-EP8 | gaattcagccagcaagacagcgatatcacctgtaagtcggacgaattcggcgctcttccgcttcctcgctca | 11 |
| chimera full | ctgactcgctgcgctcggtcgttcggctgcggcgagcggtatcagctcactcaaaggcggtaatacggttat | |
| genome | ccacagaatcaggggataacgcaggaaagaacatgtgagcaaaaggccagcaaaaggccaggaaccgtaaaa | |
| | aggccgcgttgctggcgtttttccataggctccgcccccctgacgagcatcacaaaaatcgacgctcaagtc | |
| | agaggtggcgaaacccgacaggactataaagataccaggcgtttccccctggaagctccctcgtgcgctctc | |
| | ctgttccgaccctgccgcttaccggatacctgtccgcctttctcccttcgggaagcgtggcgctttctcata | |
| | gctcacgctgtaggtatctcagttcggtgtaggtcgttcgctccaagctgggctgtgtgcacgaaccccccg | |
| | ttcagcccgaccgctgcgccttatccggtaactatcgtcttgagtccaacccggtaagacacgacttatcgc | |
| | cactggcagcagccactggtaacaggattagcagagcgaggtatgtaggcggtgctacagagttcttgaagt | |
| | ggtggcctaactacggctacactagaagaacagcatttggtatctgcgctctgctgaagccagttaccttcg | |
| | gaaaaagagttggtagctcttgatccggcaaacaaaccaccgctggtagcggtggtttttttgtttgcaagc | |
| | agcagattacgcgcagaaaaaaaggatctcaagaagatcctttgatcttttctacggggtctgacgctcagt | |
| | ggaacgaaaactcacgttaagggattttggtcatgaacctgtaagtcggacgattatcaaaaaggatcttca | |
| | cctagatccttttaaattaaaaatgaagttttaaatcaatctaaagtatatatgagtaaacttggtctgaca | |
| | gttaccaatgcttaatcagtgaggcacctatctcagcgatctgtctatttcgttcatccatagttgcctgac | |
| | tccccgtcgtgtagataactacgatacgggagggcttaccatctggccccagtgctgcaatgataccgcgag | |
| | acccacgctcaccggctccagatttatcagcaataaaccagccagccggaagggccgagcgcagaagtggtc | |
| | ctgcaactttatccgcctccatccagtctattaattgttgccgggaagctagagtaagtagttcgccagtta | |
| | atagtttgcgcaacgttgttgccattgctacaggcatcgtggtgtcacgctcgtcgtttggtatggcttcat | |
| | tcagctccggttcccaacgatcaaggcgagttacatgatcccccatgttgtgcaaaaaagcggttagctcct | |
| | tcggtcctccgatcgttgtcagaagtaagttggccgcagtgttatcactcatggttatggcagcactgcata | |
| | attctcttactgtcatgccatccgtaagatgcttttctgtgactggtgagtactcaaccaagtcattctgag | |
| | aatagtgtatgcggcgaccgagttgctcttgcccggcgtcaatacgggataataccgcgccacatagcagaa | |
| | ctttaaaagtgctcatcattggaaaacgttcttcggggcgaaaactctcaaggatcttaccgctgttgagat | |
| | ccagttcgatgtaacccactcgtgcacccaactgatcttcagcatcttttactttcaccagcgtttctgggt | |
| | gagcaaaaacaggaaggcaaaatgccgcaaaaaagggaataagggcgacacggaaatgttgaatactcatac | |
| | tcttcctttttcaatattattgaagcatttatcagggttattgtctcatgagcggatacatatttgaatgta | |
| | tttagaaaaataaacaaataggggttccgcgcacatttccccgaaaagtgccacgtgagcgatcgcatagta | |
| | atcaattacggggtcattagttcatagcccatatatggagttccgcgttacataacttacggtaaatggccc | |
| | gcctggctgaccgcccaacgacccccgcccattgacgtcaataatgacgtatgttcccatagtaacgccaat | |
| | agggactttccattgacgtcaatgggtggagtatttacggtaaactgcccacttggcagtacatcaagtgta | |
| | tcatatgccaagtacgccccctattgacgtcaatgacggtaaatggcccgcctggcattatgcccagtacat | |
| | gaccttatgggactttcctacttggcagtacatctacgtattagtcatcgctattaccatggtgatgcggtt | |
| | ttggcagtacatcaatgggcgtggatagcggtttgactcacggggatttccaagtctccaccccattgacgt | |
| | caatgggagtttgttttggcaccaaaatcaacgggactttccaaaatgtcgtaacaactccgccccattgac | |
| | gcaaatgggcggtaggcgtgtacggtgggaggtctatataagcagagctggtttagtgaaccgtcagatcca | |
| | cgtcatctgatgaggccgaaaggccgaaacggtgaaagccgtaatgacgtataggtgttggctctatgcctt | |
| | gacatttgtattgtcaggagctgtgaccattggcacagtccaaaacttgctgcgcagaaacacccttctgcg | |
| | acagcctccttcaggggagcttggggtctatccctagcaccttgcttctggagttgcactgctttacggtct | |
| | ctccacccctttaaccatgtctgggatacttgatcggtgcacgtgtacccccaatgccagggtgtttatggc | |
| | ggagggccaagtttactgcacacgatgtctcagtgcacggtctctccttcccctgaatctccaagtttctga | |
| | gctcggggtgctgggcctattctacaggcccgaagagccactccggtggacgttgccacgtgcattccccac | |
| | tgttgagtgctcccccgccggcgcatgctggctttccgctatctttccaatcgcacgaatgaccagtggaaa | |
| | cctaaacttccaacaaagaatggtacgggtcgccgccgaaatatacagagtcgggcaacttacgcccgctgt | |
| | attgaaggctctgcaagtatacgagcggggttgtaggtggtatcccatcgtaggacctgtccctggagtggc | |
| | cgttttcgccaactccctgcatgtgagtgatagacctttcccgggagcaactcacgtattaaccaacctgcc | |
| | actcccgcagagacccaagcctgaggacttttgccccttcgagtgtgctatggccgccgtatacgacgtcgg | |
| | acatggcgccgttatgtacgttgccgacgagagagtctcctgggcccctcgcggcggcgacgaagttagatt | |
| | cgaaacggtcccacaggagcttaagtcggttgcgaaccaattatgtacgtcgttcccaccccaccacgtagt | |
| | cgatatgtctaagttcgcctttaccgcccccggttgcggcgtatctatgcgggtcgaacgtcaatacggctg | |
| | tctccccgccgatacggtccccgaaggcaactgttggtggagcttgttcgattcgctcccactcgaagtgca | |
| | aggcaaagagattcgccacgctaaccaattcgggtatcagactaagcatggcgtatccggtaagtacctaca | |
| | gcgtaggctgcaaatcaacggtctccgcgcagtcgctgaccctaacggacctttcgtcgtacagtacttctc | |
| | cgtcaaggagagttggatccgccacttgaaactggccgaagaacctagttaccccgggttcgaggacctcct | |
| | ccgcataagggttgagtctaatacgtcaccattggctaacaaggacgaaaaaattttccggtttggcagtca | |
| | taagtggtacggcgctggtaagagagcaaggaaagcacgctctcgtgcgactaatacagtcgctgaccgcgc | |
| | tttgtccgttcgtgaaatctggcaggccaaggagcatgaggttaccggcgccaataaggctgagcacctcaa | |
| | acactactccccacctgccgaagggaattgtggttggcactgcatttccgccatcgtcaaccggatggttaa | |
| | ttccaaatttgaaaccacccttcccgaaagagtgagacctccagatgactgggctaccgacgaggatcttgc | |
| | gaatgccatccaaatcctcagactccctgcggccttagacaggaacggtgcttgtgctagcgccaagtacgt | |
| | acttaagctggaaggtgagcattggactgtcactgtgacccctgggatgtccccttctttgctccctcttga | |
| | atgtgttcagggctgttgtgggcacaagggcggtcttggttccccagacgcagtcgaggtctccggatttga | |
| | ccctgcctgccttgaccgtctggctgaggtgatgcacctgcctagcagtgctatcccagccgctctggccga | |
| | aatgtctggtgattccgatcgttcggcttctccggtcaccaccgtgtggactgtttcgcagttctttgcccg | |
| | tcacagcggaggaaatcaccctgaccaagtgcgcttagggaaaatcatcagcctttgtcaggtgattgagga | |
| | ctgctgctgttcccagaacaaaaccaaccgggtcaccccggaggaggttgcagcaaagattgacctgtacct | |
| | ccgcggtgcaacaaatcttgaagaatgcttggccaggctcgagaaagcgcgcccgccacgggtaatcgacac | |
| | cttctttgattgggacgttgtgctccctggggttgaggcagcaacccaaacgaccaagctgccccaggtcaa | |
| | ccagtgtcgtgctctggtccctgttgtgactcaaaagtccttggacaacaactcggtccccctgaccgcctt | |
| | ttcactggctaactactactaccgcgcgcaaggtgacgaagtttgtcaccgtgaaagacttaatgccgtgct | |
| | ttccaagctggaagaggttgtccgagaagagtacgggctcatgcctaccgggcctggcccacggcccacatt | |
| | gccacgcgggctcgacgaactcaaagaccagatggaggtggacttgctgaaactggctaacgcccaggcgac | |
| | ttctgacatgatggcttgggcagtcgagcaggttgacctaaaaacttgggtcaagaactacccgcggtggat | |
| | gccaccacctcctccgccaaaagttcggcctcgaaaaacgaagcctgtcaagagcttgccagagggaaagcc | |
| | cgcccccgcccctcgcaggagggttgggactggttgtggcagcccgacttcattgggcgatgatgttcttaa | |
| | cagttgggaagatttggctgttggcagcccctcgggtctcccgaccccccctgagccggcaacaccttcgag | |
| | tgagccagtggttgtgtcagcaccgcagtgcatcttcagaccggcgacgcccttgagtgagccgaccccaat | |
| | tcccgcaccccgtgggactgcgtctcgaccagtgacacctttgagcgagccgatccctgtgcctgcaccgcg | |
| | gcgtaaatttcagcaggcgagaaagttgagttcggcggtggtaattccgccgtaccaggacgagcccctgga | |
| | tttgtccgcttcctcgctgactgaatatgaagcccttcctctagcgccgccgcaaagcgagggcgctctaag | |
| | agtggaggggcatgaagttgaggaagccctgagtgaaatctcagacatgtcgggtggcactaaacctgcgcc | |
| | cgtatcatcaagcagctccttgtccagcgtgagaatcacacgcccaaaacactcagctcaagccatcatcga | |
| | ctcggtcgggccctgcagtgggcatctccaagaggtgaaggagatatgccttagtgtcatgcgcgaggcatg | |
| | tgatgcgactaagctcaatgaccccgctacgcaggaatggctctctcgcatgtgggatcgggtggacatgct | |
| | gacttggcgcaacacgtctgcttaccagacgcttagcaccttaggtggctggtcaaggttcctcccaaaatt | |
| | gatacttgagacaccgccgccctacccgtgtgagtttgtgatgatgccttacacgcctgcaccttccgtggg | |
| | tgcggagagcgacctcaccattggctcagttgctactgaggatgttccacgcatccttgagaacatggagaa | |
| | tgtcggcgagatgactaaccagggacccttggccttctccgaggataaaccggtggatgaccaaccttccga | |
| | agacctccggacatcgtcgcaaggacctgacgggagcacatcagctccgtccgcagatacagctggcgccgg | |
| | ctcatccaccgatttgccgtcttcaggcggcgtggatgcggacggagggggaccgcttcgggcggtaaaaag | |
| | aaaaaccgaacggctctttgaccaactgagtcgtcaggtttttgacctcgtctcccatctccctattttctt | |
| | ctcacgcctcttcaaccctggcggtggttactctccgggtgattggggttttgcagcttttactctattgtg | |
| | cctctttttatgttacagttacccagcatttggcatcgttcccctcttgggtgtattttctgggtcttctcg | |
| | gcgcgtgcgaatgggggtttttggctgctggttggcttttgctgttggtctgttcaagcctgtgtccgaccc | |
| | agtcggcgctgcttgtgagtttgactcgccagaatgcagaaacatccttcattcttttgagcttctcaaacc | |
| | ttgggaccctgttcgcagccttgttgtgggccccgtcggtctcggttttgccattcttggcaggctactggg | |
| | cggggcacgcagcatctggcactttttgcttaggcttggcattgttgcagactgtgtcttggctggagctta | |
| | tgtgctttctcaaggtaggtgcaaaaagtgttggggatcttgtataagaaccgctcctaatgaggtcgcttt | |
| | caacgtgtttccttttacgcgtgcaaccaggtcgtcacttgttgacctttgcgatcggttttgtgcgccgag | |
| | aggcatggaccccatttttctcgccactgggtggcgtggatgctgggccggtcggagccccattgagcaacc | |
| | ctctgaaaaacccatcgcgtttgcccaattggatgaaaagaagattacggctaggactgtggtcgcccagcc | |
| | ttatgatcccaaccaagccgtaaagtgcctgcgggtattgcaggcgggaggggcgatggtggctgaggcggt | |
| | cccaaaagtggtcagggtttccgctgttccatttcgagcccccttttttcctaccggagtgaaggttgaccc | |
| | tgaatgtagagtagtggttgaccctgacactttcactgcagctctccggtctggttactccaccacaaacct | |
| | cgtcctcggtgtgggggactttgcccagctgaatggattaaaaatcaggcaaatttctaagccttcaggggg | |
| | gggcccacatctcatggctgccctgcatgttgcctgctcgatgattttgcacatgtttgctgggatttatgt | |
| | gactgcggtgggttcttgcggcaccggcaccaacgatccgtggtgcgcaaacccatttgccgtccctggcta | |
| | cggacctggctccctctgcacgtccagattgtgtatttcccagcacggccttaccctgcccttgacagcact | |
| | tgtggcgggattcggcatccaggaaattgccttagtcgctttgatctttgtttccatcggaggcattgctca | |
| | taggttgagttgtaaagctgacatgctgtgtgttttgcttgcaattgccagctatgtttgggtacctcttac | |
| | ctggttactttgtgtttttccttgctggttgcgctggttttctttgcatccccttaccgtcctatggttggt | |
| | gtttttcttgatttctgtgaatatgccttcaggaatcttggccatggtgttgttagtttctctttggcttct | |
| | tggtcgttatactaatgtcgccggtcttgtcactccctacgacattcatcattacaccagtggcccccgcgg | |
| | tgttgccgcattggctactgcaccagatggaacctacttagccgctgttcgccgcgctgcgttgactggccg | |
| | caccatgttgttcaccccgtcccagcttgggtctcttcttgagggcgctttcagaactcgaaagccctcact | |
| | gaacaccgtcaatgtggtcgggtcctccatgggctctggcggggtgtttaccattgacgggaaagtcaggtg | |
| | cgtgactgccacacatgtccttacgggtaactcagccagggtttccggaaccggcttcaatcaaatgcttga | |
| | ctttgacgtaaaaggggatttcgctatagccgattgcccgaattggcaaggggctgcccccaaaacccaatt | |
| | ctgcgaggatggatggactggccgtgcctattggctaacatcctctggcgtcgaacccggcgtcattgggaa | |
| | aggattcgccttctgcttcaccgcgtgcggcgattccgggtccccagtgatcaccgaggccggtgagcttgt | |
| | cggtgttcacacgggatcgaataaacaaggggggggtattgtcacacgcccctcaggccagttttgtaatgt | |
| | ggcacccatcaagctgagcgaattaagtgaattctttgctgggcctaaggtcccgctcggtgatgtgaaggt | |
| | tggcagccacataatcaaagacacaagcgaggtgccttcagatctttgtgccttgcttgctgccaaacctga | |
| | actggaaggaggtctctccaccgtccaacttctgtgtgtgtttttccttctgtggagaatgatgggacatgc | |
| | ctggacgcccttggttgctgtgggtttctttattttgaatgagattctcccggccgtcctggtccggagtat | |
| | tttctcctttggaatgtttgtgctatcctggctcacgccatggtctgcgcaagttctgatgatcaggctcct | |
| | gacagcagctcttaacaggaatagatgttcacttgcctttttcagcctcggtgcagtgaccggttttgttgc | |
| | agatcttgcggctactcaggggcatccgttgcaggcagtgatgaatttgagcacctatgcattcctgcctcg | |
| | ggtaatggttgtgacttcaccagtcccagtgatcgcgtgtggtgttgtgcacctactcgccatcattctgta | |
| | cctgtttaaataccgtagcctgcaccatatccttgttggcgatggagtgttctcttcggccttcttcctgcg | |
| | atattttgccgaaggaaagttgagggaaggggtgtcacaatcctgcggaatgaatcatgagtctctgactgg | |
| | tgccctcgctatgagactcaatgacgaggacttggatttcctcatgaaatggactgattttaagtgcttcgt | |
| | ttctgcgtccaacatgaggaatgcagcgggtcaatttatcgaggctgcatatgctaaagcacttagagttga | |
| | actggcccagttggtgcaggttgataaggttcgaggtactatggccaaacttgaagcttttgctgatactgt | |
| | ggcacctcaactctcgcccggtgatattgttgtcgctctcggccacacgcctgttggcagtatcttcgactt | |
| | aaaggttggtagcaccaagcataccctccaagccattgagaccagggttcttgctgggtccaaaatgaccgt | |
| | ggcgcgcgtcgtcgacccgacccccacgcccccacccgcgcccgtgcccatccccctcccaccgaaagttct | |
| | ggagaatggccccaacgcttggggggatgaggaccgtttgaataagaagaaaagacgcaggatggaagccct | |
| | cggcatctatgttatgggcgggaaaaagtaccagaaattttgggacaagaactccggtgatgtgttttatga | |
| | ggaggtccataacaacacagatgagtgggagtgtctcacagttggcgaccctgccgactttgaccctgagaa | |
| | gggaaccctgtgtggacatgtcaccattgacaacaaggcttaccatgtttacacctcctcatctggtaagaa | |
| | gttcttggttcccgtcaacccagagaacggaagagttcaatgggaagctgcaaagctttccgtggagcaggc | |
| | cctaggcatgatgaatgtcgacggcgaactgactgccaaggaactggagaaactgaaaagaataattgacaa | |
| | actccagggcctgactaaggagcagtgtttaaactgctagccgccagcgacttgacccgctgtggtcgcggc | |
| | ggcttggttgttactgagacagcggtgaaaatagtcaaatttcacaatcggactttcactctggggcctgtg | |
| | aatttaaaagtggccagtgaggttgagctaaaagatgcggttgagcacaaccaacacccggttgcgaggccg | |
| | atcgatggtggagttgtgctcctgcgttccgcggttccctcgcttatagacgtcttgatctccggtgctgac | |
| | acatctcccaagttacttgcccttcacgggccgggaaacactgggatcgatggcacgctctgggatttcgag | |
| | tccgaagccactaaagaggaagtcgcactcagtgcgcaaataatacaggcttgtgacattaggcgtggcgac | |
| | gctcctgaaattggtctcccttataagctgtaccctgttaggggcaaccctgagcgggtgaaaggagttctg | |
| | cagaatacaaggtttggagacataccctacaaaacccctagtgacaccgggagcccagtgcacgcggctgcc | |
| | tgccttacgcccaacgccactcctgtgactgatgggcgttccgtcttggctacaaccatgccccccgggttt | |
| | gagttatatgtaccgaccataccagcgtctgtccttgattaccttgactctaggcctgactgccctaaacag | |
| | ctgacagagcacggctgcgaagacgccgcactgaaagacctctctaaatatgacttatctacccaaggcttt | |
| | gtcttacctggggttcttcgccttgtgcggaaatacctgtttgcccatgtaggtaaatgtccacccgttcat | |
| | cggccttctatttaccccgctaagaattctatggctggaataaatgggaacaggttcccaaccaaagacatc | |
| | cagagcgttcctgaaatcgacgttctgtgcgcacaggctgtgcgggaaaactggcaaactgtcaccccttgt | |
| | actcttaagaaacagtattgcgggaagaagaagaccaggaccatactcggcaccaataacttcattgcactg | |
| | gcccaccgagcagtgttgagtggtgtcacccagggcttcatgaaaaaggcgttcaactcgcccatcgccctc | |
| | ggaaagaacaagttcaaggagctacagactccggtcctgggcaggtgccttgaagctgacctcgcatcctgt | |
| | gatcgatccacgcctgcaattgttcgctggtttgctgccaaccttctttatgaacttgcctgtgctgaagag | |
| | tatctaccgtcgtatgtgctgaactgctgccacgacttactagtcacgcagtccggcgcagtgactaagaga | |
| | ggtggcctgtcgtctggcgacccgatcacctctgtgtctaacaccatttacagtttggtgatctatgcacag | |
| | cacatggtgcttagttactttaaaagtggtcacccccatggccttctgttcttacaagaccagctaaagttt | |
| | gaggacatgctcaaggttcaacccctgatcgtctattcggacgatctcgtgctgtatgccgagtctcccacc | |
| | atgccaaattatcactggtgggttgagcatctgaatttgatgctggggtttcagacggacccaaaaaaaaca | |
| | gcaataacagattcgccatcatttctgggctgtagaataatgaatgggcgccagctagtccccaaccgtgac | |
| | aggatcctcgcggccctcgcctatcacatgaaggcaagtaatgtttctgaatactatgcctcagcggctgca | |
| | atactcatggacagctgtgcttgtttggagtatgatcctgaatggtttgaagaacttgtagttggaatagcg | |
| | cagtgcgcccgcaaggatggctacagctttcccggcacgccgttcttcatgtccatgtgggaaaaactcagg | |
| | tccaattatgaggggaagaagtcgagagtgtgcgggtactgcggggccccggccccgtacgctactgcctgt | |
| | ggcctcgacgtctgcatctaccacacccacttccaccagcattgtccagtcacaatctggtgtggccatcca | |
| | gcgggttctggttcttgtagtgagtgcaaatcccctgtagggaaaggcacaaaccctttagacgaggtgctg | |
| | gaacaagtcccgtataagcccccacggaccgttatcatgcatgtagagcagggtctcaccccccttgatcca | |
| | ggtagataccaaactcgccgcggattagtttccgttaggcgtggaattaggggaaatgaagttgaactacca | |
| | gacggtgattatgctagcaccgccttgctccctacctgcaaagagatcaacatggtcgctgtcgcttccaat | |
| | gtattgcgcagcaggttcatcatcggcccacccggtgcagggaaaacatactggctccttcaacaggtccag | |
| | gacggtgatgttatttacacaccaactcaccagaccatgcttgacatgatcagggctttggggacgtgccgg | |
| | ttcaacgtcccggcaggcacaacgctgcaattccctgtcccctcccgcaccggtccgtgggttcgcatcctg | |
| | gccggcggttggtgtcctggcaagaattccttcctagatgaagcagcgtattgcaaccaccttgatgttttg | |
| | aggcttcttagcaaaactaccctcacctgtctaggagacttcaagcaactccacccagtgggttttgattct | |
| | cattgttatgtttttgacatcatgcctcaaactcaactgaagaccatctggaggttcggacagaatatctgt | |
| | gatgccatccagccagattacagggacaagctcatgtccatggtcaacacaacccgtgtgacctacgtggaa | |
| | agacctgtcaggtatgggcaggtcctcaccccctaccacagggaccgagaggacgacgccatcactattgac | |
| | tccagtcaaggcgccacattcgatgtggtcacattgcatttgcccactaaagattcactcaacaggcaaaga | |
| | gcccttgttgccatcaccagggctagacacgctatctttgtgtatgacccacacaggcagctgcagagcttg | |
| | tttgatcttcctgcaaaaggcacacccgtcaaccttgcagtgcaccgcgacgggcagctgatcgtacttgat | |
| | agaaataacaaagaatgcacggttgctcaggctctaggcaacggggataaattcagggccacagacaagcgt | |
| | gttgcagattcactccgcgccatttgtgctgatctagaagggtcgagttctccgctccccaaggtcgcacac | |
| | aacttggggttttatttctcacctgacttaacacaatttgccaaactcccagtggaacttgcacctcattgg | |
| | cccgtggtgacaacccagaacaatgaaaagtggccagatcggctggtcgccagccttcgccctatccataaa | |
| | tacagccgcgcgtgcatcggtgccggctatatggtgggcccttcggtgtttctaggcactcctggggtcgta | |
| | tcatactttcttacaaaatttgttaaaggcgaggctcaaatgcttccagaaacagtcttcagcaccggccga | |
| | attgaggtggattgccgggaatatcttgacgatcgggagcgggaagttgctgcatccctcccacacgccttt | |
| | attggcgacgtcaaaggcaccaccgttggcggatgtcatcatgtcacctccaggtaccttcctcgcttcctt | |
| | cccaaagagtcagttgcggtagtcggggtctcaagccccgggaaagccgcgaaggcaatgtgcacactgaca | |
| | gatgtgtacctcccagatcttgaagcctatctccacccggagacccagtccaggtgctggaaaatgatgttg | |
| | gacttcaaagaagttcgactgatggtctggaaagacaaaacggcctatttccaacttgagggtcgttatttc | |
| | acctggtatcagctcgctagttatgcctcgtacgtccgtgttcctgtcaactccacggtgtacttggacccc | |
| | tgcatgggcccagcccattgcaacagacgagttgtcgggtccactcagtggggagctgacctcgcaatcacc | |
| | ccctatgattatggcgccaagatcattttgtctagcgcatatcacggtgaaatgccccctgggtacaagatt | |
| | ctggcgtgtgcggagttctcattggatgacccggtcagatacaaacacacctggggatttgagtcggataca | |
| | gcgtacctgtacgagttcactggaaatggtgaggattgggaagattataatgatgcgtttcgtgcgcgccag | |
| | aaagggaaaatttataaggccactgccaccagcttgaagttttatttccctccgggccctgtcgttgaacca | |
| | actttgggcctgaactgaaggcgcgccgccaccatgctgtggggcgtatattccgcaatcgaaacgtggaag | |
| | ttcataacgagtcgctgccgtctctgcttattagggcgaaagtatatactcgctcctgctcaccacgtggag | |
| | tcggcttaggttccgtggcaacccctataaccagagtttcagcggaacaggcgcgccatgaaatggggtcca | |
| | tgcaaagccttttttacaaaattggccaactttttgtggatgctttcacggagttcttggtgtccattgttg | |
| | atatcattatatttttggccattttgtttggcttcaccatcgccggttggctggtggtcttttgcatcagat | |
| | tggtttgctccgcgatactccgtacgcgccctgccattcactctgagcaattacagaagatcttatgaggcc | |
| | tttctttcccagtgccaagtggacattcccacctggggaactaaacatcctttggggatgctttggcaccat | |
| | aaggtgtcaaccctgattgatgaaatggtgtcgcgtcgaatgtaccgcatcatggaaaaagcagggcaggct | |
| | gcctggaaacaggtggtgagcgaggctacgctgtctcgcattagtagtttggatgtggtggctcattttcag | |
| | catctagccgccattgaagccgagacctgtaaatatttggcctcccggctgcccatgctacacaacctgcgc | |
| | atgacaggttcaaatgtaaccatagtgtataatagcactttgaatcaggtgtttgctatttttccaacccct | |
| | ggttcccggccaaagcttcatgattttcagcaatggttaatagctgtacattcctccatattttcctctgtt | |
| | gcagcttcttgtactctttttgttgtgctgtggttgcgggttccaatactacgtactgtttttggtttccgc | |
| | tggttaggggcaatttttctttcgaactcacagtgaattacacggtgtgtccaccttgcctcacccggcaag | |
| | cagccacagagatctacgaacccggtaggtctctttggtgcaggatagggtatgaccgatgtgaggaggatg | |
| | atcatgacgagctagggtttatggtaccgcctggcctctccagcgaaggccacttgactagtgtttacgcct | |
| | ggttggcgttcttgtccttcagctacacggcccagttccatcccgagatattcgggatagggaatgtgagtc | |
| | gagtttatgttgacatcaaacatcaactcatctgcgccgaacatgacgggcagaacaccaccttgcctcgtc | |
| | atgacaacatttcagccgtgtttcagacctattaccaacatcaagtcgacggcggcaattggtttcacctag | |
| | aatggcttcgtcccttcttttcctcgtggttggttttaaatgtctcttggtttctcaggcgttcgcctgcaa | |
| | accatgtttcagttcgagtcttgcagatattaagaccaacaccaccgcagcggcaagctttgctgtcctcca | |
| | agacatcagttgccttaggcatcgcgactcggcctctgaggcgattcgcaaaatccctcagtgccgtacggc | |
| | gatagggacacccgtgtatgttaccatcacagccaatgtgacagatgagaattatttacattcttctgatct | |
| | cctcatgctttcttcttgccttttctatgcttctgagatgagtgaaaagggatttaaggtggtatttggcaa | |
| | tgtgtcaggcatcgtggctgtgtgtgtcaattttaccagctacgtccaacatgtcaaggagtttacccaacg | |
| | ctccctggtggtcgaccatgtgcggttgctccatttcatgacacctgagaccatgaggtgggcaactgtttt | |
| | agcctgtctttttgccattctgttggcaatttgaatgtttaagtatgttggagaaatgcttgaccgcgggct | |
| | gttgctcgcaattgctttctttgtggtgtatcgtgccgttctgttttgctgtgctcgccaacgccagcaacg | |
| | acagcagctcccatctacagctgatttacaacttgacgctatgtgagctgaatggcacagattggctagcta | |
| | acaaatttgattgggcagtggagagttttgtcatctttcccgttttgactcacattgtctcctatggtgccc | |
| | tcactaccagccatttccttgacacagtcgctttagtcactgtgtctaccgccgggtttgttcacgggcggt | |
| | atgtcctaagtagcatctacgcggtctgtgccctggctgcgttgacttgcttcgtcattaggtttgcaaaga | |
| | attgcatgtcctggcgctacgcgtgtaccagatataccaactttcttctggacactaagggcggactctatc | |
| | gttggcggtcgcctgtcatcatagagaaaaggggcaaagttgaggtcgaaggtcatctgatcgacctcaaaa | |
| | gagttgtgcttgatggttccgtggcaacccctataaccagagtttcagcggaacaatggggtcgtccttaga | |
| | tgacttctgtcatgatagcacggctccagaaaaggtgcttttggcgttttctattacctacacgccagtgat | |
| | gatatatgccctaaaggtgagtcgcggccgactgctagggcttctgcaccttttgatcttcctgaattgtgc | |
| | tttcaccttcgggtacatgactttcgcgcactttcagagtacaaataaggtcgcgctcactatgggagcagt | |
| | agttgcactcctttggggggtgtactcagccatagaaacctggaaattcatcacctccagatgccgtttgtg | |
| | cttgctaggccgcaagtacattctggcccctgcccaccacgttgaaagtgccgcaggctttcatccgattgc | |
| | ggcaaatgataaccacgcatttgtcgtccggcgtcccggctccactacggtcaacggcacattggtgcccgg | |
| | gttaaaaagcctcgtgttgggtggcagaaaagctgttaaacagggagtggtaaaccttgtcaaatatgccaa | |
| | ataacaacggcaagcagcagaagagaaagaagggggatggccagccagtcaatcagctgtgccagatgctgg | |
| | gtaagatcatcgctcagcaaaaccagtccagaggcaagggaccgggaaagaaaaataagaagaaaaacccgg | |
| | agaagccccattttcctctagcgactgaagatgatgtcagacatcactttacccctagtgagcggcaattgt | |
| | gtctgtcgtcaatccagaccgcctttaatcaaggcgctgggacttgcaccctgtcagattcagggaggataa | |
| | gttacactgtggagtttagtttgcctacgcatcatactgtgcgcctgatccgcgtcacagcatcaccctcag | |
| | catgatgggctggcattcttgaggcatctcagtgtttgaattggaagaatgtgtggtgaatggcactgattg | |
| | acattgtgcctctaagtcacctattcaattagggcgaccgtgtgggggtgagatttaattggcgagaaccat | |
| | gcggccgaaattaaaaaaaaaaaaaaaaaaaaaaaaggccggcatggtcccagcctcctcgctggcgccggc | |
| | tgggcaacatgcttcggcatggcgaatgggacttaattaa | |

(In Tables 1 and 2 above, the polynucleotide provided in the present description may comprise a nucleic acid sequence in which T in the described sequence is substituted with U, and in Table 2, the nucleic acid sequences of the T cell epitope-EP7 (Modified T cell epitope-EP7) and/or T cell epitope-EP8 (Modified T cell epitope-EP8) introduced, respectively, are underlined)

The chimeric virus of PRRSV comprising the polynucleotide of the Structural formula 1 as genome may comprise progeny viruses cultured for 1 to 80 passages, 1 to 70 passages, 1 to 60 passages, 1 to 50 passages, 1 to 40 passages, 1 to 30 passages, 1 to 20 passages, 1 to 10 passages, or 1 to 5 passages.

One specific embodiment provides the chimeric virus of PRRSV comprising a nucleic acid sequence comprising the polynucleotide of SEQ ID NO: 6 as genome, and this was named PRRSV-EP7. The PRRSV-EP7 virus may be a virus with accession number KCTC 14269BP.

One specific embodiment provides the chimeric virus of PRRSV comprising a nucleic acid sequence comprising the polynucleotide of SEQ ID NO: 11 as genome, and this was named rPRRSV-EP8. The rPRRSV-EP8 virus may be a virus with accession number KCTC 14270BP.

In addition, other embodiment provides a vector comprising the polynucleotide of Structural formula 1 described above (e.g., genome of the chimeric virus mutant strain of PRRSV). The vector may be recombinantly obtained, and it may be used as a carrier or expression vector of the polynucleotide.

Other embodiment may provide a cell comprising the polynucleotide (e.g., genome; RNA, DNA, or both of them of chimeric virus of PRRSV) of Structural formula 1 described above, or a vector comprising thereof. The cell may be recombinantly obtained, and it means a cell in which the genome (RNA, DNA, or a vector comprising thereof) of the chimeric virus mutant strain, or the chimeric virus mutant strain comprising the genome, is transfected to prepare a large amount of the chimeric virus mutant strain, and as long as it is within the target range, the type of the cell is not particularly limited.

### Use of Chimeric Virus Mutant Strain

Other embodiment provides a porcine reproductive and respiratory syndrome virus vaccine composition, comprising at least one selected from the group consisting of the polynucleotide of Structural formula 1, a vector comprising the same, and the chimeric virus mutant strain of PRRSV comprising the polynucleotide of Structural formula 1 as genome.

Other embodiment provides a pharmaceutical composition for prevention and/or treatment of porcine reproductive and respiratory syndrome, comprising at least one selected from the group consisting of the polynucleotide of Structural formula 1, a vector comprising the same, and the chimeric virus mutant strain of PRRSV comprising the polynucleotide of Structural formula 1 as genome. The porcine reproductive and respiratory syndrome may be caused by a Porcine Reproductive and Respiratory Syndrome Virus (PRRSV).

Other embodiment provides a method for immunization against a porcine reproductive and respiratory syndrome virus, or prevention and/or treatment of porcine reproductive and respiratory syndrome infection or porcine reproductive and respiratory syndrome, comprising administering a pharmaceutically effective dose of at least one selected from the group consisting of the polynucleotide of Structural formula 1, a vector comprising the same, and the chimeric virus mutant strain of PRRSV comprising the polynucleotide of Structural formula 1 as genome into a subject in need of immunization against a porcine reproductive and respiratory syndrome virus, or prevention and/or treatment of porcine reproductive and respiratory syndrome infection or porcine reproductive and respiratory syndrome. The subject may be an animal except for humans, and for example, it may be a pig.

The chimeric virus mutants train of PRRSV may comprise subcultured progeny. The subcultured progeny may include progeny viruses cultured for 1 to 80 passages, 1 to 70 passages, 1 to 60 passages, 1 to 50 passages, 1 to 40 passages, 1 to 30 passages, 1 to 20 passages, 1 to 10 passages, or 1 to 5 passages.

According to one embodiment, the vaccine may be a live vaccine or killed vaccine, or it is preferable to be a live vaccine. Specifically, the attenuated PRRS chimeric virus mutant strain described herein may be a modified live vaccine containing at least one virus strain described above in a pharmaceutically acceptable carrier in a survived state. In addition, or alternatively, an inactivated virus may be used for preparing a killed vaccine.

The vaccine may further comprise at least one selected from the group consisting of a carrier, a diluent, an excipient, and an adjuvant. The type of the pharmaceutically acceptable carrier is not particularly limited, but it may comprise any all solvents, dispersion media, coat ings, stabilizers, preservatives, antibacterial agents and antifungal agents, isotonic agents, absorption delaying agents, and the like.

The genome of the PRRSV-EP7 chimeric virus mutant strain provided in the present description, and the vaccine composition comprising the same may be used to prevent pigs from effects of PRRS disease. In addition, subunits in addition to immunogenic fragments or fractions of the rPRRSV-EP7 chimeric virus mutant strain may be also used for preventing pigs from effects of PRRS disease. The attenuated chimeric virus mutant strain of the present invention or the vaccine composition comprising the same may be preventively administered before pigs are exposed to a PRRS virus strain causing PRRS, and it may be administered into pigs at the same time as pigs are exposed to the virus strain, and it may be therapeutically administered after target pigs are exposed to the virus strain.

The genome of the PRRSV-EP8 chimeric virus mutant strain provided in the present description, and the vaccine composition comprising the same may be used to prevent pigs from effects of PRRS disease. In addition, subunits in addition to immunogenic fragments or fractions of the rPRRSV-EP8 chimeric virus mutant strain may be also used for preventing pigs from effects of PRRS disease. The attenuated chimeric virus mutant strain of the present invention or the vaccine composition comprising the same may be preventively administered before pigs are exposed to a PRRS virus strain causing PRRS, and it may be administered into pigs at the same time as pigs are exposed to the virus strain, and it may be therapeutically administered after target pigs are exposed to the virus strain.

The vaccine composition provided in the present description may be used for prevention of Porcine Reproductive and Respiratory Syndrome (PRRS) and/or Porcine Reproductive and Respiratory Syndrome Virus (PRRSV) infection, for example, infection of North American PRRSV and/or European PRRSV and/or PRRS caused thereby. In one embodiment, the North American PRRSV may be a type II VR2332 virus, and the European PRRSV may be a type I virus (Lelystad virus, LV), but not limited thereto.

The pharmaceutical composition for prevention and/or treatment of a Porcine Reproductive and Respiratory Syndrome Virus (PRRSV) described above may comprise an additional component known in the art, and it may further comprise a carrier, an excipient and a diluent suitable, commonly used for preparation of pharmaceutical compositions.

In addition, according to conventional methods, it may be used as being formulated in a form of oral formulations such as powders, granules, tablets, capsules, suspensions, emulsions, syrups, aerosols and the like, external preparations, suppositories and sterile injection solutions. It is preferable to use those disclosed in the literature (Remington's Pharmaceutical Science, recent, Mack Publishing Company, Easton PA) as suitable preparations known in the art.

The carrier, excipient and diluent which can be comprised in the pharmaceutical composition includes lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxy benzoate, propyl hydroxy benzoate, talc, magnesium stearate and mineral oil and the like. When the composition is formulated, it is prepared using a diluent or excipient such as a filler, an extender, a binder, a wetting agent, a disintegrating agent, a surfactant and the like. Solid preparations for oral administration include tablets, pills, powders, granules, capsules and the like, and such solid preparations are prepared by mixing at least one or more excipients, for example, starch, calcium carbonate, sucrose, lactose, gelatin and the like. In addition, in addition to simple excipients, lubricants such as magnesium stearate, and talc are also used. Suspensions, oral liquids, emulsions, syrups and the like correspond to liquid preparations for oral administration, and in addition to simple diluents, water and liquid paraffin, various excipients, for example, a wetting agent, a sweetener, a flavoring agent, a preservative and the like may be comprised. Preparations for parenteral administration include sterilized aqueous solutions, non-aqueous solvents, suspending agents, emulsions, freeze-dried preparations, and suppositories. As the non-aqueous solvents and suspending agents, propylene glycol, polyethylene glycol, vegetable oils such as olive oil, injectable ester such as ethyl oleate and the like may be used. As a base of the suppositories, Witepsol, Macrogol, Tween61, cacao butter, laurin better, glycerogelatin and the like may be used.

A preferable dose of the pharmaceutical composition may vary depending on the subject's condition and body weight, degree of disease, drug form, administration route and period, but it may be appropriately selected by those skilled in the art.

For example, for a preferable effect, the composition of the present invention may be administered in an amount of 0.0001 to 1,000 mg/kg (body weight) per 1 day. Administration of the composition may be administering once a day, or may be administering by dividing into several times. In one embodiment, the pharmaceutical composition may be a vaccine composition. The vaccine may be a live vaccine and/or a killed vaccine, and specifically, the attenuated PRRS chimeric virus described herein may be a modified live vaccine containing at least one virus strain described above into a pharmaceutically acceptable carrier in a survived state. In addition, or alternatively, an inactivated virus may be used for preparing a killed vaccine.

The vaccine may comprise a PRRSV mutant strain at an appropriate concentration in consideration of the body weight, age, dietary step and/or immunity of a subject for administration in a range of a preventive purpose of PRRS. For example, the dose of the virus mutant strain in the vaccine composition may be in a range of TCID₅₀ 2 to 6, or TCID₅₀ 3 to 4, but it may vary depending on the type of the subject, but not limited thereto.

The composition may be administered in a range of TCID₅₀ (Median Tissue Culture Infectious Dose; virus concentration which infects 50% of cells) 2 to 6, or TCID₅₀ 3 to 4, based on the PRRSV-EP7 chimeric virus mutant strain content, and may vary depending on the type of the subject, but not limited thereto. The administration of the composition may be administering once a day, and may be administering by dividing into several times.

The composition may be administered in a range of TCID₅₀ (Median Tissue Culture Infectious Dose; virus concentration which infects 50% of cells) 2 to 6, or TCID₅₀ 3 to 4, based on the PRRSV-EP8 chimeric virus mutant strain content, and may vary depending on the type of the subject, but not limited thereto. The administration of the composition may be administering once a day, and may be administering by dividing into several times.

The composition of the present invention may be administered into a subject through various routes. All the methods of administration may be expected.

The chimeric virus mutant strain of PRRSV or composition comprising the same provided in the present description mya be administered through oral, or parenteral administration such as subcutaneous, intramuscular, intradermal, sublingual, transdermal, intra-rectal, transmucosal, surface area through inhalation, or buccal administration or the like, or a combination thereof. Furthermore, the PRRS chimeric virus mutant strain may be administered in a transplant form capable of allowing sustained release of the virus. In other embodiment, the chimeric virus mutant strain or composition comprising thereof may be administered by subcutaneous injection, intravenous injection, intradermal injection, parenteral injection, intramuscular injection, needle free injection, electroporation, oral delivery, intranasal delivery, oronasal delivery, or any combination thereof.

In one specific embodiment, the chimeric virus mutant strain of PRRSV or composition comprising thereof may be administered through injection, inhalation or transplantation, and injection is particularly preferable. According to the desired period and effectiveness of vaccination or treatment, the chimeric virus or composition comprising thereof may be administered once or several times, in addition, intermittently, for example, in other doses daily for several days, several weeks or several months. The injection may be injected in a desired amount, or may be injected by spraying into a nasal cavity, or alternatively, may be continuously injected.

### Coadministration

In one specific embodiment, at least one selected from the group consisting of the polynucleotide represented by the following Structural formula 2, a vector comprising thereof, and a chimeric virus mutant strain of PRRSV comprising the polynucleotide of Structural formula 2 as genome, and
at least one selected from the group consisting of the polynucleotide represented by the following Structural formula 3, a vector comprising thereof, and a chimeric virus mutant strain of PRRSV comprising the polynucleotide of Structural formula 3 as genome may be used together (coadministered):

[Structural formula 2]
5′-[X]-[Ya]-[Z]-3′
[Structural formula 3]
5′-[X]-[Yb]-[Z]-3′

In the Structural formulas 2 and 3, [X] is same as Structural formula 1 described above, and may comprise a nucleic acid sequence (for example, SEQ ID NO: 2) of the NSP1 gene (NSP1-alpha gene and NSP1-beta gene; ORF1a and ORF1b) of a PRRS type II virus, for example, LMY parent strain (Accession No. GenBank accession no. DQ473474.1) or LMY ver2 virus with accession number KCTC 13394BP, or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) within a range of maintaining equivalent functions to the nucleic acid sequence, and for example, the nucleic acid sequence of the NSP1 gene of the LMY ver2 virus may be represented by SEQ ID NO: 2, and the nucleic acid sequence of the NSP1 gene of the LMY parent strain may be represented by SEQ ID NO: 1.

The [Ya] may be a nucleic acid sequence encoding T cell epitope-EP7 (LLAFSITYTPVMIYALKVSRGRLLGL; SEQ ID NO: 3), which is a PRRSV-derived membrane protein, or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the nucleic acid sequence while maintaining functional equivalency thereto, and specifically, the EP7-encoding nucleic acid sequence may be the nucleic acid sequence of SEQ ID NO: 4 or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the sequence of SEQ ID NO: 4 while maintaining functional equivalency thereto.

In addition, the [Ya] may be a modified EP7-encoding nucleic acid sequence. The modified EP7-encoding nucleic acid sequence, may further comprise at least one selected from the group consisting of AscI restriction enzyme recognition site (SEQ ID NO: 8), Kozak sequence (SEQ ID NO: 13) and a transcription regulatory sequence (e.g., TRS6 (SEQ ID NO: 14), in addition to the EP7-encoding nucleic acid sequence. In one embodiment, the AscI restriction enzyme recognition site may be comprised at both ends of the EP7-encoding nucleic acid sequence, respectively. In one embodiment, the Kozak sequence and/or transcription regulatory sequence may be each independently comprised at the 5′ end and/or 3′ end of the EP7-encoding nucleic acid sequence, for example, at the 5′ end for the Kozak sequence, and the 3′ end for the transcription regulatory sequence (TRS6).

In one specific embodiment, the [Ya] may comprise an EP7-encoding nucleic acid sequence (e.g., SEQ ID NO: 4), Kozak sequence positioned at the 5′ end of the EP7-encoding nucleic acid sequence (e.g., SEQ ID NO: 13), TRS6 positioned at the 3′ end of the EP7-encoding nucleic acid sequence (e.g., SEQ ID NO: 14), and the AscI restriction enzyme recognition site positioned at the 5′ end of the Kozak sequence and the 3′ end of the TRS6, respectively (SEQ ID NO: 8), and specifically, it may be the nucleic acid sequence of SEQ ID NO: 20 or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the nucleic acid sequence of SEQ ID NO: 20 while maintaining functional equivalency thereto.

The [Yb] may be a nucleic acid sequence encoding T cell epitope-EP8 (LWGVYSAIETWKFITSRCRLCLLGRKYILAPAHHVESA; SEQ ID NO: 12), which is a PRRSV-derived membrane protein, or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the nucleic acid sequence while maintaining functional equivalency thereto, and specifically, the EP8-encoding nucleic acid sequence may be the nucleic acid sequence of SEQ ID NO: 10 or a nucleic acid sequence having sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the sequence of SEQ ID NO: 10 while maintaining functional equivalency thereto.

In addition, the [Yb] may be a modified EP8-encoding nucleic acid sequence. The modified EP8-encoding nucleic acid sequence, may further comprise at least one selected from the group consisting of AscI restriction enzyme recognition site (SEQ ID NO: 8), Kozak sequence (SEQ ID NO: 13) and a transcription regulatory sequence (e.g., TRS6 (SEQ ID NO: 14), in addition to the EP8-encoding nucleic acid sequence. In one embodiment, the AscI restriction enzyme recognition site may be comprised at both ends of the EP8-encoding nucleic acid sequence, respectively. In one embodiment, the Kozak sequence and/or transcription regulatory sequence may be each independently comprised at the 5′ end and/or 3′ end of the EP8-encoding nucleic acid sequence, for example, at the 5′ end for the Kozak sequence, and the 3′ end for the transcription regulatory sequence (TRS6).

In one specific embodiment, the [Yb] may comprise an EP8-encoding nucleic acid sequence (e.g., SEQ ID NO: 10), Kozak sequence positioned at the 5′ end of the EP8-encoding nucleic acid sequence (e.g., SEQ ID NO: 13), TRS6 positioned at the 3′ end of the EP8-encoding nucleic acid sequence (e.g., SEQ ID NO: 14), and the AscI restriction enzyme recognition site positioned at the 5′ end of the Kozak sequence and the 3′ end of the TRS6, respectively (SEQ ID NO: 8), and specifically, it may be the nucleic acid sequence of SEQ ID NO: 21 or a nucleic acid sequence having a sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) to the nucleic acid sequence of SEQ ID NO: 21 while maintaining functional equivalency thereto.

The [Z] is same as Structural formula 1 described above, and in other words, it may be a gene nucleic acid sequence of ORF2 to ORF7 regions of the BP2017-2 virus with accession number KCTC 13393BP (for example, SEQ ID NO: 5) or a nucleic acid sequence having a sequence identity of 70% or more (or 75% or more, 80% or more, 85% or more, 90% or more, 91% or more, 92% or more, 93% or more, 94% or more, 95% or more, 96% or more, 97% or more, 98% or more, 99% or more, or 99.5% or more; hereinafter, applied in the same way) within a range of maintaining equivalent functions to the nucleic acid sequence.

Therefore, one embodiment
provides a porcine reproductive and respiratory syndrome virus vaccine composition, comprising
at least one selected from the group consisting of the polynucleotide of Structural formula 2, a vector comprising the same, and a chimeric virus mutant strain of PRRSV comprising the polynucleotide of Structural formula 1 as genome, and
at least one selected from the group consisting of the polynucleotide of Structural formula 3, a vector comprising the same, and a chimeric virus mutant strain of PRRSV comprising the polynucleotide of Structural formula 2 as genome.

Another embodiment
provides a composition for prevention and/or treatment of porcine reproductive and respiratory syndrome, comprising
at least one selected from the group consisting of the polynucleotide of Structural formula 2, a vector comprising the same, and a chimeric virus mutant strain of PRRSV comprising the polynucleotide of Structural formula 1 as genome, and
at least one selected from the group consisting of the polynucleotide of Structural formula 3, a vector comprising the same, and a chimeric virus mutant strain of PRRSV comprising the polynucleotide of Structural formula 2 as genome. The porcine reproductive and respiratory syndrome may be caused by a Porcine Reproductive and Respiratory Syndrome Virus (PRRSV).

Another embodiment
provides a method for immunization against a porcine reproductive and respiratory syndrome virus, or prevention of porcine reproductive and respiratory syndrome virus infection or prevention of porcine reproductive and respiratory syndrome, comprising co-administering a medically and pharmaceutically effective dose of
at least one selected from the group consisting of the polynucleotide of Structural formula 2, a vector comprising the same, and a chimeric virus mutant strain of PRRSV comprising the polynucleotide of Structural formula 1 as genome, and
at least one selected from the group consisting of the polynucleotide of Structural formula 3, a vector comprising the same, and a chimeric virus mutant strain of PRRSV comprising the polynucleotide of Structural formula 2 as genome
into a subject in need of immunization against porcine reproductive and respiratory syndrome virus, or prevention of porcine reproductive and respiratory syndrome virus infection or prevention of porcine reproductive and respiratory syndrome. The subject may be an animal other than humans, and for example, it may be a pig.

The chimeric virus of PRRSV and/or mutant strain thereof may comprise subcultured progeny. The subcultured progeny may include a progeny virus cultured for 1 to 80 passages, 1 to 70 passages, 1 to 60 passages, 1 to 50 passages, 1 to 40 passages, 1 to 30 passages, 1 to 20 passages, 1 to 10 passages, or 1 to 5 passages.

According to one embodiment, the PRRSV-EP7 chimeric virus may express cytokines such as IFN-gamma and the like or increase expression by reacting with blood of a person to be inoculated, for example, peripheral blood mononuclear cells (PBMCs). When PBMCs are stimulated with the PRRSV-EP7 chimeric virus or vaccine composition comprising thereof, IFN-gamma may be expressed more by about 1.05 times or more, about 1.1 times or more, about 1.3 times or more, 1.5 times or more, about 1.7 times or more, about 1.9 times or more, or about 2.1 times or more, and may be expressed more by about 2.5 times or less, about 3.0 times or less, about 3.5 times or less, compared to the genome of the chimeric virus of PRRSV in which the EP7 gene is not inserted, and vaccine composition comprising the same.

According to one embodiment, the PRRSV-EP7 chimeric virus may stimulate porcine PBMCs infected with PRRS. When porcine PBMCs infected with PRRS are stimulated with the vaccine composition, the amount of the infected PRRSV may be reduced by about 0.7 times or less, about 0.5 times or less, about 0.4 times or less, about 0.3 times or less, or about 0.2 times to about 0.3 times, compared to PBMCs without any stimulation.

According to one embodiment, the PRRSV-EP8 chimeric virus may express cytokines such as IFN-gamma and the like or increase expression by reacting with blood of a person to be inoculated, for example, peripheral blood mononuclear cells (PBMCs). When PBMCs are stimulated with the PRRSV-EP8 chimeric virus or vaccine composition comprising thereof, IFN-gamma may be expressed more by about 1.1 times or more, about 1.3 times or more, 1.5 times or more, about 1.7 times or more, about 1.9 times or more, or about 2.1 times or more, and may be expressed more by about 2.5 times or less, about 3.0 times or less, about 3.5 times or less, compared to the genome of the chimeric virus of PRRSV in which the EP8 gene is not inserted, and a vaccine composition comprising the same.

According to one embodiment, the PRRSV-EP8 chimeric virus may stimulate porcine PBMCs infected with PRRS. When porcine PBMCs infected with PRRS are stimulated with the vaccine composition, the amount of the infected PRRSV may be reduced by about 0.7 times or less, about 0.5 times or less, about 0.4 times or less, about 0.3 times or less, or about 0.2 times to about 0.3 times, compared to PBMCs without any stimulation.

Another embodiment,
may provide a method for producing a chimeric virus mutant strain of porcine reproductive and respiratory syndrome virus, comprising
(1) preparing a polynucleotide fragment by treating genome of LMY ver2 virus with accession number 13394BP or LMY parent strain (Accession No. GenBank accession no. DQ473474.1) with restriction enzyme (e.g., AscI and/or PacI); (2) preparing a polynucleotide fragment comprising T cell epitope-EP7 and/or T cell epitope-EP8, which are PRRSV-derived membrane proteins;
(3) preparing a polynucleotide fragment by treating genome of BP2017-2 virus with accession number KCTC 13393BP with restriction enzyme (e.g., AscI and/or PacI); and
(4) preparing an infectious clone by recombining the polynucleotide fragments obtained from the steps (1) to (3). The steps (1) to (3) may be performed at the same time or sequentially regardless of the order.

In one embodiment, the step (2) may be performed by chemically or recombinantly synthesizing the following nucleic acid sequence, or treating both ends of genome of PRRSV-EP7 virus with AscI. In case of AcsI treatment, the obtained polynucleotide fragment may comprise the EP7-encoding nucleic acid sequence (for example, SEQ ID NO: 4), and AscI recognition site (SEQ ID NO: 8) at both ends of the EP7-encoding nucleic acid sequence, respectively. In addition, the step (2) may additionally comprise a step of inserting kozak sequence (SEQ ID NO: 13) at the front (5′ end) of the EP7-encoding nucleic acid sequence, and TRS6 (SEQ ID NO: 14) at the back (3′ end). In this case, the polynucleotide fragment obtained in the step (2) may comprise the EP7-encoding nucleic acid sequence (SEQ ID NO: 4), Kozak sequence positioned at the 5′ end of the EP7-encoding nucleic acid sequence (SEQ ID NO: 13), TRS6 positioned at the 3′ end of the EP7-encoding nucleic acid sequence (SEQ ID NO: 14), and AscI restriction enzyme recognition site positioned at the 5′ end of the Kozak sequence and the 3′ end of the TRS6, respectively (SEQ ID NO: 8), and specifically, it may be represented by the nucleic acid sequence of SEQ ID NO: 20.

In one embodiment, the step (2) may be performed by chemically or recombinantly synthesizing the following nucleic acid sequence, or treating both ends of genome of PRRSV-EP8 virus with AscI. In case of AcsI treatment, the obtained polynucleotide fragment may comprise the EP8-encoding nucleic acid sequence (for example, SEQ ID NO: 10), and AscI recognition site (SEQ ID NO: 8) at both ends of the EP8-encoding nucleic acid sequence, respectively. In addition, the step (2) may additionally comprise a step of inserting kozak sequence (SEQ ID NO: 13) at the front (5′ end) of the EP8-encoding nucleic acid sequence, and TRS6 (SEQ ID NO: 14) at the back (3′ end). In this case, the polynucleotide fragment obtained in the step (2) may comprise the EP8-encoding nucleic acid sequence (SEQ ID NO: 10), Kozak sequence positioned at the 5′ end of the EP8-encoding nucleic acid sequence (SEQ ID NO: 13), TRS6 positioned at the 3′ end of the EP8-encoding nucleic acid sequence (SEQ ID NO: 14), and AscI restriction enzyme recognition site positioned at the 5′ end of the Kozak sequence and the 3′ end of the TRS6, respectively (SEQ ID NO: 8), and specifically, it may be represented by the nucleic acid sequence of SEQ ID NO: 21.

The polynucleotide fragment prepared by treating the genome of the LMY ver2 virus or LMY parent strain of the step (1) with restriction enzymes AscI and PacI may comprise a region encoding NSP1-beta (ORF1a and ORF1b; SEQ ID NO: 2 or SEQ ID NO: 1).

The polynucleotide fragment prepared by treating the genome of the BP2017-2 virus of the step (3) with restriction enzymes AscI and PacI may comprise ORF2 to ORF7 regions (SEQ ID NO: 5).

In the recombining the polynucleotide fragments of the step (4), two kinds or more, or all of the three kinds of the polynucleotide fragments of the (1) to (3) may be recombined at the same time or sequentially regardless of the order, and the recombination may be performed using a ligase. The infectious clone obtained by the recombination may be inoculated into cells to prepare the chimeric virus.

The cell means a cell in which chimeric virus DNA or RNA, or a vector, an infectious clone, or a chimeric virus, which comprises the same, is transfected, to prepare a large amount of the chimeric virus, but the type of the cell is not particularly limited.

Another embodiment may provide a kit for performing any immunization, prevention and/or treatment method. This kit may comprise a container, preferably, a vaccine composition containing the chimeric virus of PRRSV described above, a pharmaceutically acceptable carrier, a reinforcing agent, and instructions for administering the immunogenic composition to an animal in need thereof to alleviate clinical signs or effects of PRRS infection, preferably, the frequency or severity of PRRS. The kit may also comprise injection means and/or other types of administration means. Moreover, the kit may comprise a solvent. The attenuated vaccine may be lyophilized, and may be a solution for injection and/or inhalation as being restored with the solvent. The solvent may be water, physiological saline solution, buffer or a reinforcing solvent. The kit may comprise an isolated container containing the attenuated virus, solvent and/or pharmaceutically acceptable carrier. The instructions may be a label and/or printed material attached to at least one container.

### Advantageous Effects

The PRRSV-EP7 and/or PRRSV-EP8 chimeric viruses provided in the present description stimulates porcine PBMCs infected with PRRS to induce expression of IFN-gamma and inhibits virus amplification in target cells, and therefore, they can effectively protect against it, so they can be usefully used as a vaccine for preventing and/or treating PRRS.

### BRIEF DESCRIPTION OF THE DRAWINGS

FIG. 1 shows a schematic diagram of PRRS infectious clones equipped with PRRS T cell peptide epitope-EP7 or T cell peptide epitope-EP8 (PRRS T cell peptide epitope-EP7 or T cell peptide epitope-EP8 is indicated as PRRSV peptide).

FIG. 2 shows peptide sequences of virus genes extracted after performing subculturing rPRRSV-EP7 equipped with PRRS T cell peptide epitope-EP7 once, 4 times, 6 times and 29 times.

FIG. 3 shows a graph capable of confirming the ability to induce IFN-gamma expression by comparing induction of IFN-gamma expression of a conventional vaccine strain and a peptide expression vaccine strain (rPRRSV-EP7).

FIG. 4 shows a graph of Virus Suppression Assay (VSA) of a conventional vaccine strain and a peptide expression vaccine strain (rPRRSV-EP7).

FIG. 5 shows peptide sequences of virus genes extracted after performing subculturing rPRRSV-EP8 equipped with PRRS T cell peptide epitope-EP8 once, 6 times and 29 times.

FIG. 6 shows a graph capable of confirming the ability to induce IFN-gamma expression by comparing induction of IFN-gamma expression of a conventional vaccine strain and a peptide expression vaccine strain (rPRRSV-EP8).

FIG. 7 shows a graph of Virus Suppression Assay (VSA) of a conventional vaccine strain and a peptide expression vaccine strain (rPRRSV-EP8).

FIG. 8 shows a graph of the result of measuring the number of cells expressing IFN-gamma through ELIspot IFN-gamma measurement according to Example 6-2.

### MODE FOR INVENTION

Hereinafter, the present invention will be described in detail by examples. However, the following examples illustrate the present invention only, but the present invention is not limited by the following examples.

Since RNA is easily destroyed, it was converted into DNA, and after performing all work, RNA was synthesized therefrom to transform cells.

### Example 1: Preparation of a Chimeric Virus

Referring to the method described in the example of Korean Patent Publication No. 2020-0081225 (incorporated as a reference in the present description), a Porcine Reproductive and Respiratory Syndrome (PRRS) chimeric virus was prepared as follows.

### Example 1-1. Design of a Chimeric Virus

A Porcine Reproductive and Respiratory Syndrome (PRRS) chimeric virus was designed to comprise a non-structural protein (Non-Structural Protein 1, NSP1; SEQ ID NO: 1) of a mutant strain of LMY strain and ORF2, ORF3, ORF4, ORF5, ORF6, and ORF7 regions of BP2017-2 isolated from Namsan Farm in Namsan-ri, Gongju-si, Chungcheongnam-do, in 2017.

### Example 1-2. Preparation of Attenuated LMY Mutant Strain

Using LMY strain (GenBank accession no. DQ473474.1.), a PRRS strain isolated at the Animal and Plant Quarantine Agency, 91 bases in the base sequence of the gene of the NSP1 region were replaced to prepare a recombinant LMY ver2 virus, an LMY mutant strain (LMY ver2 mutant strain). In the gene of the NSP1 region, 25 bases in the NSP1-alpha region, and 66 bases in the NSP1-beta region were substituted.

Specifically, the recombinant LMY ver2 virus was prepared by silent mutation of some of the base sequence according to Codon Pair Deoptimization principle (Table 3) using commonly known SAVE (Synthetic Attenuated Virus Engineering) 1 program. At first, using the SAVE program, the CPB (codon pair base) value, which is a bias that occurs when the gene codons of the LMY virus interact when they are arranged in pairs, was quantified using a computer algorithm. The CPB value fluctuates when some base sequences of the LMY virus gene is substituted with other base sequences, and is closely related to proliferation of the virus. In the proliferation of the virus, when the CPB value is reduced (deoptimized) through base sequence substitution, the proliferation is reduced and attenuated (Virus Attenuation by Genome-Scale Changes in Codon Pair Bias, Science, 2008, J. Robert Coleman et al.). The present inventors selected NSP1 region (NSP1-alpha and NSP1-beta, SEQ ID NO: 1) with high genetic stability in genome of the LMY parent strain, and analyzed it with the SAVE program, and among base regions, a total of 91 bases, 25 in the NSP1-alpha region, 66 in the NSP1-beta region, were selected, and substituted with other bases and deoptimized to prepare an LMY virus mutant strain. The LMY virus mutant strain in which 91 base sequences of NSP1 prepared by the above method were mutated (SEQ ID NO: 2) was referred to as LMY ver2, and the base sequence was shown in Table 3 below. The LMY ver2 has accession number 13394BP.

TABLE 3

| | | SEQ ID |
|---|---|---|
| Name | Base Sequence (5′ -> 3′) | NO |
| NSP1 gene | atgtctgggatacttgatcggtgcacgtgtacccccaatgccagggtgtttatggcggagggccaagt | 1 |
| of LMY | ttactgcacacgatgtctcagtgcacggtctctccttcccctgaatctccaagtttctgagctcgggg | |
| (NSP1-beta | tgctgggcctattctacaggcccgaagagccactccggtggacgttgccacgtgcattccccactgtt | |
| region is | gagtgctcccccgccggggcctgctggctttctgcaatctttccaattgcacgaatgaccagtggaaa | |
| indicated | cctaaacttccaacaaagaatggtacgggtcgcagctgagatctacagagttggccagctcacccctg | |
| in italics) | cagttttgaaggctctacaagtttatgagcggggttgccgctggtatcccattgttggacctgtccct | |
| | ggagtggccgttttcgccaactccctgcatgtgagtgatagacctttcccgggagcaactcacgtatt | |
| | aaccaacctgccactcccgcagagacccaagcctgaggacttttgccccttcgagtgtgctatggctg | |
| | ctgtctacgatgttggtcatggcgccgtcatgtatgtggccgatgagagagtctcctgggcccctcgt | |
| | ggcggggatgaagtaagatttgaaactgtcccacaggagctcaagtcggttgcgaaccaactctgcac | |
| | ctccttcccaccccaccacgtagtggacatgtctaagttcgcctttacagcccctgggtgtggtgttt | |
| | ctatgcgggtcgaacgtcaatatggctgtctccccgctgacactgtccccgaaggcaactgctggtgg | |
| | agcttgtttgactcgctcccattggaagtccagggcaaagaaattcgccatgctaaccaatttggcta | |
| | ccagaccaagcatggtgtctctggtaagtacctacagcggaggctgcaaattaatggtctccgagcag | |
| | tagctgacccaaatggacctttcgtcgtacagtacttctccgtcaaggagagttggatccgccacttg | |
| | aaactagcggaagaacccagttaccctgggtttgaggacctcctcagaataagggttgagtctaacac | |
| | gtcaccattggctaacaaggatgaaaaaattttccggtttggcagtcataagtggtacggc | |
| NSP1 gene | atgtctgggatacttgatcggtgcacgtgtacccccaatgccagggtgtttatggcggagggccaagt | 2 |
| of LMY ver2 | ttactgcacacgatgtctcagtgcacggtctctccttcccctgaatctccaagtttctgagctcgggg | |
| (NSP1-beta | tgctgggcctattctacaggcccgaagagccactccggtggacgttgccacgtgcattccccactgtt | |
| region is | gagtgctcccccgccggcgcatgctggctttccgctatctttccaatcgcacgaatgaccagtggaaa | |
| indicated | cctaaacttccaacaaagaatggtacgggtcgccgccgaaatatacagagtcgggcaacttacgcccg | |
| in italics) | ctgtattgaaggctctgcaagtatacgagcggggttgtaggtggtatcccatcgtaggacctgtccct | |
| | ggagtggccgttttcgccaactccctgcatgtgagtgatagacctttcccgggagcaactcacgtatt | |
| | aaccaacctgccactcccgcagagacccaagcctgaggacttttgccccttcgagtgtgctatggccg | |
| | ccgtatacgacgtcggacatggcgccgttatgtacgttgccgacgagagagtctcctgggcccctcgc | |
| | ggcggcgacgaagttagattcgaaacggtcccacaggagcttaagtcggttgcgaaccaattatgtac | |
| | gtcgttcccaccccaccacgtagtcgatatgtctaagttcgcctttaccgcccccggttgcggcgtat | |
| | ctatgcgggtcgaacgtcaatacggctgtctccccgccgatacggtccccgaaggcaactgttggtgg | |
| | agcttgttcgattcgctcccactcgaagtgcaaggcaaagagattcgccacgctaaccaattcgggta | |
| | tcagactaagcatggcgtatccggtaagtacctacagcgtaggctgcaaatcaacggtctccgcgcag | |
| | tcgctgaccctaacggacctttcgtcgtacagtacttctccgtcaaggagagttggatccgccacttg | |
| | aaactggccgaagaacctagttaccccgggttcgaggacctcctccgcataagggttgagtctaatac | |
| | gtcaccattggctaacaaggacgaaaaaattttccggtttggcagtcataagtggtacggc | |

(In Table 3 above, the underlined part of the nucleic acid sequence of the LMY ver2 NSP1 gene is the base portion in which a mutation occurred in the LMY NSP1 gene)

Then, in order to confirm the attenuation level of the LMY ver2 mutant strain, the CPB value was measured. The NSP1 CPB value of the LMY parent strain was measured to be about 0.0139, and the NSP1-beta CPB value was measured to be about 0.016, but the CPB value of the LMY ver2 mutant strain of the present invention was measured to be about-0.2393 in the NSP1, and about-0.33 in the NSP1-beta. From this, it could be seen that the proliferation of the LMY ver2 mutant strain of the present invention was reduced than the conventional parent strain, and it was an attenuated strain. Detailed contents related thereto were shown in Table 4.

TABLE 4

| | | Codon Pare |
|---|---|---|
| Virus strain | NSP1 region | Deoptimization (CPB) |
| LMY | NSP1 | 0.01392516640897059 |
| LMY ver2 | NSP1 | −0.2393184052058128 |
| LMY | NSP1-beta | 0.016079236408108866 |
| LMY ver2 | NSP1-beta | −0.3377269630038442 |

Then, an infectious clone of the LMY ver2 was prepared.

At first, the entire gene sequence (GenBank accession no. DQ473474.1.) of the LMY strain were synthesized by dividing into 7 fragments, respectively. Fragment 1 among the 7 fragments was NPS-1 region, and the region was synthesized as a DNA fragment (SEQ ID NO: 2) in which 91 bases among the gene base sequence of the NSP1 region of the LMY strain were substituted. The synthesized fragment genes were cut with restriction enzymes of Table 5 below in order, and then linked with ligase to prepare one infectious clone.

TABLE 5

| | | | | Restriction |
|---|---|---|---|---|
| | | | | enzyme |
| | Restriction | Tm | | recognition |
| Fragment | enzyme | (° C.) | Buffer | site |
| 1 | AsiSI | 37 | Cut Smart | GCGATCGC |
| | XhoI | 37 | Cut Smart | CTCGAG |
| 2 | XhoI | 37 | NEB3.1 | CTCGAG |
| | MluI | 37 | NEB3.1 | ACGCGT |
| 3 | MluI | 37 | NEB3.1 | ACGCGT |
| | Nsil | 37 | NEB3.1 | ATGCAT |
| 4 | Nsil | 37 | Cut Smart | ATGCAT |
| | SpeI | 37 | Cut Smart | ACTAGT |
| 5 | SpeI | 37 | Cut Smart | ACTAGT |
| | AscI | 37 | Cut Smart | GGCGCGCC |
| 6 | AscI | 37 | Cut Smart | GGCGCGCC |
| | AfeI (blunt) | 37 | Cut Smart | AGCGCT |
| 7 | AfeI (blunt) | 37 | Cut Smart | AGCGCT |
| | PacI | 37 | Cut Smart | TTAATTAA |

### Example 1-3. Construction of Chimeric Virus Clone

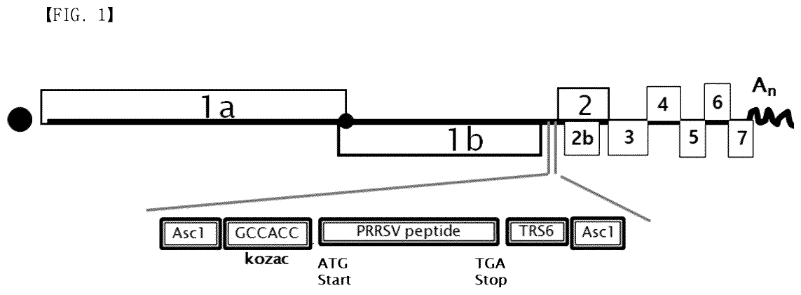

As shown in the schematic diagram of FIG. 1, the PRRS virus comprises a total of 8 ORFs, which are ORF1a, ORF1b, and ORF2 to 7.

In the genomic region comprising the entire structural genes of the LMY ver2 mutant strain (infectious clone of LMY ver2) constructed in Example 1-2 above, ORF2 to ORF7 regions were cut using restriction enzymes, AscI and PacI. Subsequently, the portion corresponding to the ORF2 to ORF7 of the LMY ver2 mutant strain in the genomic region of the BP2017-2 strain (SEQ ID NO: 5) was cut with the same AscI and PacI restriction enzymes, and then the portion corresponding to the ORF1a and OFR1b regions of the LMY ver2 mutant strain (LMY ver2 NSP1 region of Example 1-2; SEQ ID NO: 2) and the portion corresponding to the ORF2 to ORF7 regions of the BP2017-2 strain (SEQ ID NO: 5) were linked with ligase to prepare a recombinant infectious clone, and named LMY+BP2017. The used restriction enzymes were shown in Table 6 below.

TABLE 6

| | | | | Restriction | |
|---|---|---|---|---|---|
| | | | | enzyme | SEQ |
| | Restriction | Tm | | recognition | ID |
| Fragment | enzyme | (° C.) | Buffer | site | NO: |
| ORF2-7 | AscI | 37 | Cut Smart | GGCGCGCC | 8 |
| | PacI | 37 | Cut Smart | TTAATTAA | 9 |

The completed infectious clone was inserted into a high copy vector equipped with a CMV promoter and an ampicillin resistant gene, and then transformed into BHK cells (Korean Cell Line Bank) using lipofectamine, and finally, an LMY+BP2017 chimeric virus comprising the ORF1a and ORF1b regions of the LMY ver2 mutant strain (LMY ver2 NSP1 region) and the ORF2 to ORF7 regions of the BP2017-2 strain was constructed.

The LMY+BP2017 chimeric virus was deposited in Korean Collection for Type Cultures of Korea Research Institute of Bioscience and Biotechnology on Oct. 24, 2018 and received accession number KCTC13675BP.

### Example 2. Preparation of Chimeric Virus Mutant Strains Equipped with T Cell Peptide Epitope

### Example 2-1. Preparation of Chimeric Virus Mutant Strain Equipped with T Cell Peptide Epitope-EP7

A T cell peptide epitope-EP7 (T cell epitope-EP7; EP7) is a part of an amino acid sequence of membrane protein (ORF6) of a PRRS virus, and synthesis of the EP7-epitope in pTOP Blunt V2 was requested to Macrogen, and this can induce production and/or secretion of interferon-gamma (IFN-gamma). The amino acid sequence of EP7 and the nucleic acid sequence of the gene encoding this were shown in Table 7 below.

TABLE 7

| | | SEQ |
|---|---|---|
| | Amino acid sequence (N→C) or | ID |
| | base sequence (5′→3′) | NO: |
| T cell peptide | LLAFSITYTPVMIYALKVSRGRLLGL | 3 |
| epitope-EP7 | | |
| T cell epitope-EP7- | ctgctagctttcagtatcacatatactccagttat | 4 |
| encoding nucleic | gatatacgcacttaaggtctctcgtgggcgtctcc | |
| acid sequence | tagggctc | |
| Modified T-cell | ggcgcgccgccaccatgctgctagetttcagtatc | 20 |
| peptide epitope- | acatatactccagttatgatatacgcacttaaggt | |
| EP7 encoding gene | ctctcgtgggcgtctcctagggctctaggttccgt | |
| | ggcaacccctataaccagagtttcagcggaacagg | |
| | cgcgcc | |

(In Table 7, the AscI restriction enzyme recognition site is underlined, and the Kozak sequence is in italics, and TRS6 is underlined and italicized, respectively, and the region between the Kozak sequence and TRS6 is the EP7-encoding nucleic acid sequence, and ATG (start codon) and TAG (stop codon) are attached and inserted back and forth of the EP7 nucleic acid sequence).

An EP7-expresing chimeric virus mutant strain (rPRRSV-EP7 chimeric virus) comprising the ORF1 region of the infectious clone of LMY ver2 prepared in Example 1 (SEQ ID NO: 2) and the region corresponding to the ORF2 to ORF7 regions of the BP2017-2 strain (SEQ ID NO: 5), and the T cell peptide epitope-EP7 (T cell epitope-EP7)-encoding gene by linking them with ligase was constructed.

Specifically, to insert the T cell peptide epitope-EP7 into a vector, a modified EP7-epitope was fragmented by treating restriction enzyme AscI into a modified EP7-epitope plasmid in pTOP Blunt V2 synthesized by requesting to Macrogen, to obtain a polynucleotide fragment comprising the T cell peptide epitope-EP7 and the AscI restriction enzyme recognition site (SEQ ID NO: 8) at both ends thereof, and the kozak sequence (SEQ ID NO: 13) at the 5′ end of the polynucleotide fragment and TRS6 (SEQ ID NO: 14) at the 3′ end were inserted additionally to construct a modified T cell peptide epitope-EP7-encoding gene (SEQ ID NO: 20).

The AscI restriction enzyme was treated to genome comprising the entire structural genes of the LMY ver2 mutant strain (infectious clone of LMY ver2) described in Example 1-3 above (present in the AscI restriction enzyme recognition site) at 37° C. for 1 hour to cut between ORF1b and ORF2, and CIP (Calf-intestinal alkaline phosphatase) enzyme was treated at 37° C. for 30 minutes, and an inactivation process was performed at 75° C. for 10 minutes to remove a phosphate group, and after gel elution, PCR purification was performed to prepare fragments. Subsequently, both ends of the gene sequence (SEQ ID NO: 20) of the T cell peptide epitope-EP7 constructed above were treated with AscI to fragment them, and in the same manner, after gel elution, PCR purification was performed to prepare T cell peptide epitope-EP7-encoding gene fragments.

The two fragments prepared through the above process were linked with ligase at 16° C. for 1 hour, and transformed into DH5alpha com cells, and after 24 hours, colonies were confirmed, and then for the colonies, PCR was performed to confirm the insert site. In addition, the ligation-confirmed colonies were grown in an LB culture medium for 24 hours, and then a plasmid was secured by the manufacturer's midi prep method using QIAGEN Plasmid Midi Kit (cat. nos. 12143), to prepare a recombinant infectious clone.

For virus construction, the infectious clone was transfected into BHK cells (Korean Cell Line Bank) using lipofectamine, and finally, a chimeric virus mutant strain comprising the ORF1a and ORF1b regions of the LMY ver2 mutant strain (LMY ver2 NSP1 region) (SEQ ID NO: 2), the T cell peptide epitope-EP7-encoding region (SEQ ID NO: 20), and ORF2 to ORF7 regions of the BP2017-2 strain (SEQ ID NO: 5) (rPRRSV-EP7 chimeric virus) was prepared. The genomic sequence of the chimeric virus mutant strain obtained in this way was shown in SEQ ID NO: 6. In addition, the chimeric virus mutant strain (rPRRSV-EP7 chimeric virus) was deposited to Korean Collection for Type Cultures of Korea Research Institute of Bioscience and Biotechnology, Biological Resources Center, Jeongeup-si, Jeollabuk-do, Korea on Aug. 10, 2020 and received accession number KCTC 14269BP.

### Example 2-2. Preparation of Chimeric Virus Mutant Strain Equipped with T Cell Peptide Epitope-EP8

PRRSV-derived T cell peptide epitope-EP8 (T cell epitope-EP8; EP8) is a part of an amino acid sequence of membrane protein (ORF6) of a PRRS virus, and synthesis of the EP8-epitope in pTOP Blunt V2 was requested to Macrogen, and this can induce production and/or secretion of interferon-gamma (IFN-gamma). The amino acid sequence of EP8 and the nucleic acid sequence of the gene encoding this were shown in Table 8 below.

TABLE 8

| | | SEQ |
|---|---|---|
| | Amino acid sequence (N→C) or | ID |
| Peptide | base sequence (5′→3′) | NO: |
| T cell peptide | LWGVYSAIETWKFITSRCRLCLLGRKYILAPAHHVESA | 12 |
| epitope-EP8 | | |
| T cell epitope- | ctgtggggcgtatattccgcaatcgaaacgtggaagtt | 10 |
| EP8-encoding | cataacgagtcgctgccgtctctgcttattagggcgaa | |
| nucleic acid | agtatatactcgctcctgctcaccacgtggagtcggct | |
| sequence | | |
| Modified T cell | ggcgcgccgccaccatgctgtggggcgtatattccgca | 21 |
| peptide epitope- | atcgaaacgtggaagttcataacgagtcgctgccgtct | |
| EP8-encoding | ctgcttattagggcgaaagtatatactcgctcctgctc | |
| gene | accacgtggagtcggcttaggttccgtggcaaccccta | |
| | taaccagagtttcagcggaacaggcgcgcc | |

(In Table 8, the AscI restriction enzyme recognition site is underlined, and the Kozak sequence is in italics, and TRS6 is underlined and italicized, respectively, and the region between the Kozak sequence and TRS6 is the EP8-encoding nucleic acid sequence, and ATG (start codon) and TAG (stop codon) are attached and inserted back and forth of the EP8 nucleic acid sequence).

An EP8-expresing chimeric virus mutant strain (rPRRSV-EP8 chimeric virus) comprising the ORF1 region of the infectious clone of LMY ver2 prepared in Example 1 (SEQ ID NO: 2) and the region corresponding to the ORF2 to ORF7 regions of the BP2017-2 strain (SEQ ID NO: 5), and the T cell peptide epitope-EP8 (T cell epitope-EP8)-encoding gene by linking them with ligase was constructed.

Specifically, to insert the T cell peptide epitope-EP8 into a vector, a modified EP8-epitope was fragmented by treating restriction enzyme AscI into a modified EP8-epitope plasmid in pTOP Blunt V2 synthesized by requesting to Macrogen, to obtain a polynucleotide fragment comprising the T cell peptide epitope-EP8 and the AscI restriction enzyme recognition site (SEQ ID NO: 8) at both ends thereof, and the kozak sequence (SEQ ID NO: 13) at the 5′ end of the polynucleotide fragment and TRS6 (SEQ ID NO: 14) at the 3′ end were inserted additionally to construct a modified T cell peptide epitope-EP8-encoding gene (SEQ ID NO: 21).

The AscI restriction enzyme was treated to genome comprising the entire structural genes of the LMY ver2 mutant strain (infectious clone of LMY ver2) described in Example 1-3 above (present in the AscI restriction enzyme recognition site) at 37° C. for 1 hour to cut between ORF1b and ORF2, and CIP enzyme was treated at 37° C. for 30 minutes, and an inactivation process was performed at 75° C. for 10 minutes to remove a phosphate group, and after gel elution, PCR purification was performed to prepare fragments. Subsequently, both ends of the gene sequence (SEQ ID NO: 21) of the T cell peptide epitope-EP8 constructed above were treated with AscI to fragment them, and in the same manner, after gel elution, PCR purification was performed to prepare T cell peptide epitope-EP8-encoding gene fragments.

The two fragments prepared through the above process were linked with ligase at 16° C. for 1 hour, and transformed into DH5alpha com cells, and after 24 hours, colonies were confirmed, and then for the colonies, PCR was performed to confirm the insert site. In addition, the ligation-confirmed colonies were grown in an LB culture medium for 24 hours, and then a plasmid was secured by the manufacturer's midi prep method using QIAGEN Plasmid Midi Kit (cat. nos. 12143), to prepare a recombinant infectious clone.

For virus construction, the infectious clone was transfected into BHK cells (Korean Cell Line Bank) using lipofectamine, and finally, a chimeric virus mutant strain comprising the ORF1a and ORF1b regions of the LMY ver2 mutant strain (LMY ver2 NSP1 region) (SEQ ID NO: 2), the T cell peptide epitope-EP8-encoding region (SEQ ID NO: 21), and ORF2 to ORF7 regions of the BP2017-2 strain (SEQ ID NO: 5) (rPRRSV-EP8 chimeric virus) was prepared. The genomic sequence of the chimeric virus mutant strain obtained in this way was shown in SEQ ID NO: 11. In addition, the chimeric virus mutant strain (rPRRSV-EP8 chimeric virus) was deposited to Korean Collection for Type Cultures of Korea Research Institute of Bioscience and Biotechnology, Biological Resources Center, Jeongeup-si, Jeollabuk-do, Korea on Aug. 10, 2020 and received accession number KCTC 14270BP.

### Example 3. Cell Passage Research

When the EP7-expressing chimeric virus mutant strain (rPRRSV-EP7 chimeric virus) prepared in Example 2 above was passaged, it was confirmed that how long the modified region (inserted gene) was maintained.

Specifically, the rPRRSV-EP7 chimeric virus was stabilized by performing subculturing 1, 4, 6 and 29 times in MARC-145 cells (KVCC) known as a PRRS virus water-soluble cell line, and after extracting genes from the virus, RT-PCR was performed to confirm the nucleic acid sequence of the inserted gene.

Specifically, using the primer set of Table 10 below and one step RT-PCR kit (Qiagen), according to the manufacturer's instructions, RT-PCR was performed, and specific PCR conditions were shown in Table 9.

TABLE 9
PCR reaction

| PCR reaction solution | | |
|---|---|---|
| composition | | Amount |
| 5× Qiagen one stepRT-PCR | | 10 ul |
| buffer | | |
| dNTP Mix10 mm | | 2 ul |
| Forward Primer | | 1 ul |
| Reverse Primer | | 1 ul |
| Qiagen one step RT-PCR | | 2 ul |
| Enzyme Mix | | |
| Template RNA | | 2 ul |
| Distilled water | | 7 ul |
| Total volume | | 25 ul |
| PCR reaction conditions | | |
| Reverse transcription | 30 min | 50° C. |
| Initial PCR | 15 min | 95° C. |
| activation step | | |
| 3-step cycling | | |
| Denaturation | 1 min | 94° C. |
| Annealing | 1 min | 67° C. |
| Extension | 1 min | 72° C. |
| Number of cycles: 30 | | |
| Final extension | 10 min | 72° C. |

TABLE 10

| | Nucleic acid | SEQ |
|---|---|---|
| | sequence | ID |
| Primer | (5′ → 3′) | NO |
| 2-Vector- | gggaagattata | 15 |
| seq-FOR | atgatgcgtttc | |
| primer | gtg | |
| PRRS-ORF2- | accagccaaccg | 16 |
| REV primer | gcgatggt | |

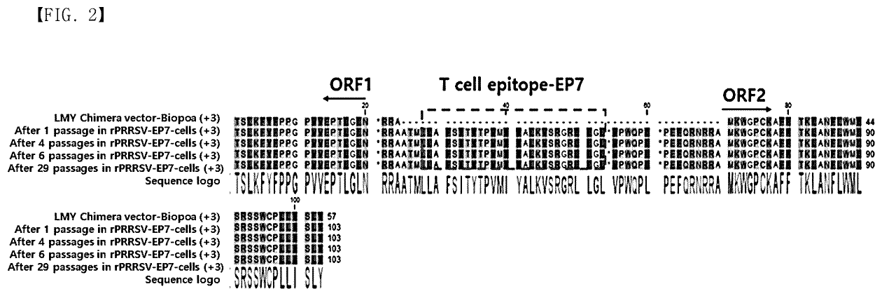

The amino acid sequence obtained based on the confirmed nucleic acid sequence was shown in FIG. 2. As shown in FIG. 2, it was confirmed that the EP7-encoding gene inserted also during the 29th subculturing was stably maintained to stably express EP7, and the result was shown in FIG. 2.

### Example 4. Confirmation of an Effect of Enhancing Immunity of rPRRSV-EP7 Chimeric Virus (In Vitro)

The effect of enhancing immunity of the rPRRSV-EP7 chimeric virus (Example 2) confirmed to stably express EP7 peptide in Example 3 above was evaluated by the level of IFN-gamma expression. For this, ELISpot (Enzyme linked immunospot) kit (MABTECH) was used.

Specifically, after the PRRS LMY virus was challenged into 3-week-old piglets (10^{4.5} TCID₅₀/ml), in each well comprising PBMCs (peripheral blood mononuclear cells) (#90-5w) 5*10⁵ cells/mL isolated after 5 weeks of infection, as a vaccine strain for antigen stimulation, rPRRSV-EP7 expressing EP7 peptide (Example 2) or rPRRSV (LMY+BP2017 chimeric virus of Example 1-3) and Inactivated LMY (LMY obtained by culturing the PRRS LMY parent strain (Accession No. GenBank accession no. DQ473474.1) for 7 passages in MARC cells were added, respectively, and they were reacted at 5% CO₂, 37° C. for 21 hours. After that, the biotin-labeled anti-IFN-gamma primary antibody and streptavidin-HRP-labeled secondary antibody (all of the above antibodies are included in ELISpot kit (MABTECH)) were sequentially treated and cultured at 37° C. for 1 hour, and then the spot forming unit (SFU) per 5*10⁵ cells was calculated. At this time, the SFU level indicates the IFN-gamma expression level.

As a control group, one in which Inactivated LMY obtained by 7 passages of the PRRS LMY parent strain (Accession No. GenBank accession no. DQ473474.1) in MARC cells was inoculated into wells comprising the PBMCs, and the PBMCs (negative control, NC) in which the vaccine strain was not inoculated were additionally prepared.

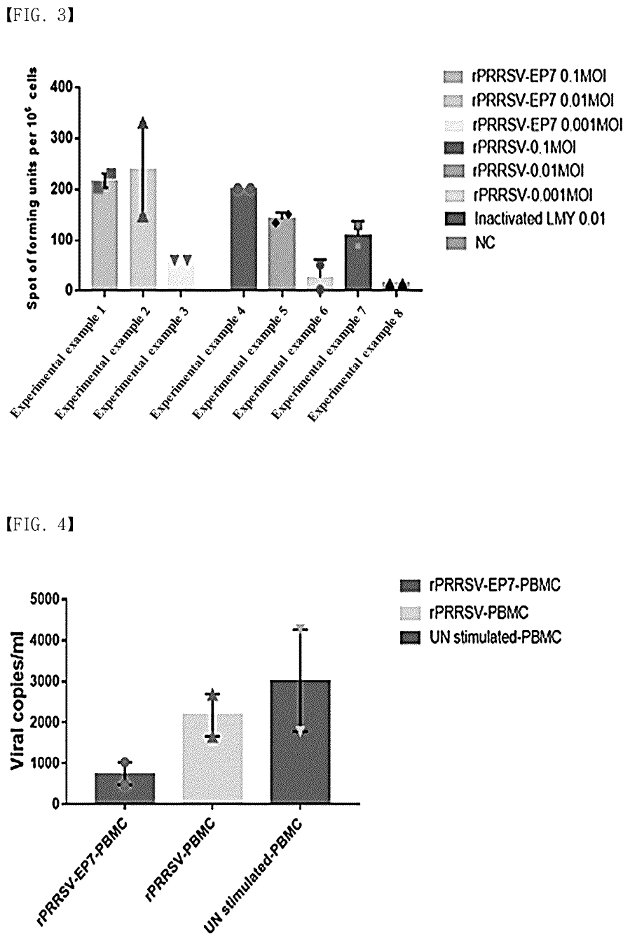

The obtained result was shown in Table 11 and FIG. 3.

TABLE 11

| | | | spot forming |
|---|---|---|---|
| | | | unit |
| | | | (SFU) per |
| | | | 5*10⁵ cells |
| | | MOI | (mean ± |
| | | (Multiplicity | standard |
| | Vaccine strain | of Infection) | deviation) |
| Experimental | rPRRSV-EP7 | 0.1 | 217 ± 14 |
| example 1 | | | |
| Experimental | rPRRSV-EP7 | 0.01 | 239.5 ± 91.5 |
| example 2 | | | |
| Experimental | rPRRSV-EP7 | 0.001 | 60 ± 0 |
| example 3 | | | |
| Experimental | rPRRSV | 0.1 | 202 ± 0 |
| example 4 | | | |
| Experimental | rPRRSV | 0.01 | 142.5 ± 8.5 |
| example 5 | | | |
| Experimental | rPRRSV | 0.001 | 26.5 ± 24.5 |
| example 6 | | | |
| Experimental | Inactivated | 0.01 | 108.5 ± 20.8 |
| example 7 | LMY | | |
| Experimental | NC | | 14 ± 0 |
| example 8 | | | |

As shown in Table 11 and FIG. 3, at the same MOI, compared to Inactivated LMY (virus supernatant with inactivated infectivity by reacting virus supernatant obtained by 7 passages in the MARC cells at 56° C. for 30 minutes) obtained by 7 passages of the rPRRSV in which the EP7 gene was not inserted, PRRS LMY parent strain (Accession No. GenBank accession no. DQ473474.1), in MARC cells and NC, the rPRRSV-EP7 expressing EP7 peptide showed a higher IFN-gamma expression level, and this result shows that a more excellent effect of enhancing immunity can be obtained as the vaccine strain expresses the EP7 peptide.

### Example 5. Confirmation of Virus Inhibition Activity of rPRRSV-EP7 Chimeric Virus

The ability of inhibiting the virus by stimulating immunocytes of the (EP7 gene-inserted) rPRRSV-EP7 chimeric virus expressing the EP7 peptide was verified using Virus Suppression Assay (VSA).

Specifically, 3-week-old piglets were infected with PRRS type 2 LMY virus (Accession No. GenBank accession no. DQ473474.1) and then porcine PBMCs (T cells) were isolated and frozen at 5 weeks after the infection. After dissolving the PBMCs, they were released to RPMI-1640 (Life Technologies, Carlsbad, CA, USA) medium in which 10% FBS (Fetal Bovine Serum) was added, and they were added to each well in an amount of 1.25×10\hat{ }6 cells, and then Type 2 PRRS chimeric virus expressing EP7 peptide inactivated with heat (rPRRSV-EP7; Example 2) or rPRRSV (Example 1-3) was cultured at 1.25*10⁴ TCID₅₀ per well, respectively, by stimulating or without stimulating for 6 days. Cells obtained in this way were used as effector cells.

In addition, target cells (monocyte derived macrophages, MDMs) differentiated from monocytes in PBMCs of an autologous or genotype-matched heterologous were prepared. More specifically, the frozen PBMCs were dissolved and then washed with serum-free EMEM (Eagle's Minimum Essential Medium) and cultured in serum-free EMEM at 37° C. for 2 hours, and then the medium and suspension cells were discarded and only adherent cells were cultured in 10% FBS-added RPMI-1640 (Life Technologies, Carlsbad, CA, USA) medium for 6 days, and then rat M-CSF (macrophage-colony stimulating factor, Biolegend, San Diego, CA, USA) was added to be 5 ng/ml every 2 days, and differentiated for 6 days to prepare VSA target cells (target MDM cells) (2.5×10\hat{ }5 cells/well). After culturing for 6 days, the effector cells activated by the stimuli prepared above and MDM cells and PRRS LMY (0.00005moi (moi based on MDM cells)) were mixed and cultured. After 4 days of the mixed culture, RNA was extracted using the cells and supernatant and the virus proliferation level was confirmed in the target cells using Real time PCR (QuantiTect Probe RT-PCR Kit; Qiagen).

Specifically, RNA 1 ul was extracted from the mixed and cultured sample supernatant 0.1 ml. The extracted RNA 1 ul was added to a mixture in which QuantiTect Probe RT-PCR buffer 5 ul and Taq man probe and 0.3 ul each of 10 pmol of the PRRSV specific primer set in Table 13 below were mixed, and 2.4 ul of DEPC distilled water was added and they were mixed to perform a real time PCR reaction. The primers used in the real-time PCR were shown in Table 13, and the PCR conditions were shown in Table 12.

TABLE 12
PCR reaction solution

| PCR reaction | | | |
|---|---|---|---|
| solution composition | | Amount | |
| Quant iTect Probe | | 5 | ul |
| RT-PCR buffer | | | |
| Enzyme | | 0.5 | ul |
| Forward Primer | | 0.3 | ul |
| Reverse Primer | | 0.3 | ul |
| Probe | | 0.5 | ul |
| Template RNA | | 1 | ul |
| Distilled water | | 2.4 | ul |
| Total volume | | 10 | ul |
| PCR reaction conditions | | | |
| Reverse transcription | 30 min | 50° | C. |
| Initial PCR activation step | 15 min | 95° | C. |
| 3-step cycling | | | |
| Denaturation | 15 sec | 94° | C. |
| Annealing | 1 min | 60° | C. |
| Extension | 1 min | 72° | C. |
| Number of cycles: 40 | | | |
| Final extension | 10 min | 72° | C. |

TABLE 13

| | | SEQ |
|---|---|---|
| | Base sequence | ID |
| Primer | (5′ → 3′ ) | NO: |
| PRRSV | atgatgrgctggc | 17 |
| specific | taact | |
| Primer | | |
| For | | |
| PRRSV | acaccggtcgccc | 18 |
| specific | taattg | |
| Primer | | |
| Rev | | |
| Taq man | 5FAM-tgtggtga | 19 |
| Probe | atggcactgattg | |
| | aca-3BHQ1 | |

The real-time PCR repeated a total of 35 cycle reactions, and Ct values by each sample was calculated according to the threshold value, and those over 35 were excluded. The Ct values were indicated by converting them into genomic copies by substituting them into the standard curve.

The obtained result was shown in FIG. 4. As shown in FIG. 4, it was confirmed that the virus reduction rate for PBMCs (T cells) without any stimulation was higher in case of PBMC stimulation by the Type 2 PRRS chimeric virus vaccine strain (rPRRSV-EP7) expressing EP7 peptide, compared to the case stimulated with the conventional vaccine strain (rPRRSV) (rPRRSV-EP7: 75%; r PRRSV: 28%). This result shows that the inhibition ability of virus proliferation was significantly increased as the vaccine strain expresses the EP7 peptide.

### Example 6. Cell Passage Research

When the EP8-expressing chimeric virus mutant strain (rPRRSV-EP8 chimeric virus) prepared in Example 2 above was passaged, it was confirmed that how long the modified region (inserted gene) was maintained.

Specifically, the rPRRSV-EP8 chimeric virus was stabilized by performing subculturing 1, 4, 6 and 29 times in MARC-145 cells (KVCC) known as a PRRS virus water-soluble cell line, and after extracting genes from the virus, RT-PCR was performed to confirm the nucleic acid sequence of the inserted gene.

Specifically, using the primer set of Table 15 below and one step RT-PCR kit (Qiagen), according to the manufacturer's instructions, RT-PCR was performed, and specific PCR conditions were shown in Table 14.

TABLE 14
PCR reaction solution

| PCR reaction | | |
|---|---|---|
| solution composition | | Amount |
| 5× Qiagen one step | | 10 ul |
| RT-PCR buffer | | |
| dNTP Mix10 mM | | 2 ul |
| Forward Primer | | 1 ul |
| Reverse Primer | | 1 ul |
| Qiagen one step | | 2 ul |
| RT-PCR Enzyme Mix | | |
| Template RNA | | 2 ul |
| Distilled water | | 7 ul |
| Total volume | | 25 ul |
| PCR reaction conditions | | |
| Reverse transcription | 30 min | 50° C. |
| Initial PCR activation | 15 min | 95° C. |
| step | | |
| 3-step cycling | | |
| Denaturation | 1 min | 94° C. |
| Annealing | 1 min | 67° C. |
| Extension | 1 min | 72° C. |
| Number of cycles: 30 | | |
| Final extension | 10 min | 72° C. |

TABLE 15

| | | SEQ |
|---|---|---|
| | Base sequence | ID |
| Primer | (5′ → 3′) | NO: |
| 2-Vector- | GGGAAGATTATAA | 15 |
| seq-FOR | TGATGCGTTTCGT | |
| primer | G | |
| PRRS-ORF2- | ACCAGCCAACCGG | 16 |
| REV primer | CGATGGT | |

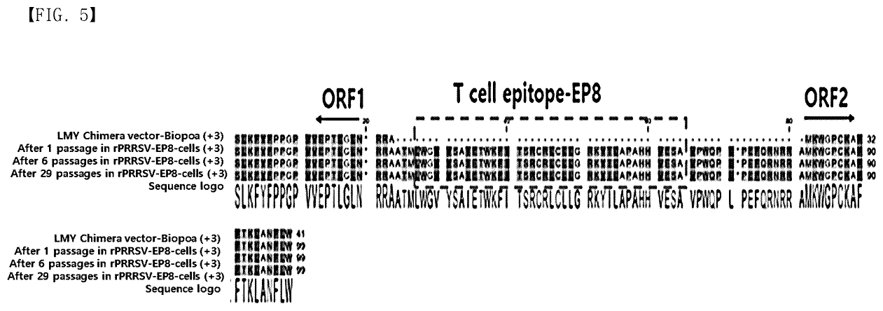

The amino acid sequence obtained based on the confirmed nucleic acid sequence was shown in FIG. 5. As shown in FIG. 5, it was confirmed that the EP8-encoding gene inserted also during the 29th subculturing was stably maintained to stably express EP8, and the result was shown in FIG. 5.

### Example 7. Confirmation of an Effect of Enhancing Immunity of rPRRSV-EP8 Chimeric Virus (In Vitro)

The effect of enhancing immunity of the rPRRSV-EP8 chimeric virus (Example 2) confirmed to stably express EP8 peptide in Example 6 above was evaluated by the level of IFN-gamma expression. For this, ELISpot (Enzyme linked immunospot) kit (MABTECH) was used.

Specifically, after the PRRS LMY virus was challenged into 3-week-old piglets (10^{4.5} TCID₅₀/ml), in each well comprising PBMCs (peripheral blood mononuclear cells) (#90-5w) 5*10⁵ cells/mL isolated after 5 weeks of infection, as a vaccine strain for antigen stimulation, rPRRSV-EP8 expressing EP8 peptide (Example 2) or rPRRSV (LMY+BP2017 chimeric virus of Example 1-3) and Inactivated LMY (LMY obtained by culturing the PRRS LMY parent strain (Accession No. GenBank accession no. DQ473474.1) for 7 passages in MARC cells were added, respectively, and they were reacted at 5% CO₂, 37° C. for 21 hours. After that, the biotin-labeled anti-IFN-gamma primary antibody and streptavidin-HRP-labeled secondary antibody (all of the above antibodies are included in ELISpot kit (MABTECH)) were sequentially treated and cultured at 37° C. for 1 hour, and then the spot forming unit (SFU) per 5*10⁵ cells was calculated. At this time, the SFU level indicates the IFN-gamma expression level.

As a control group, one in which Inactivated LMY obtained by 7 passages of the PRRS LMY parent strain (Accession No. GenBank accession no. DQ473474.1) in MARC cells was inoculated into wells comprising the PBMCs was prepared.

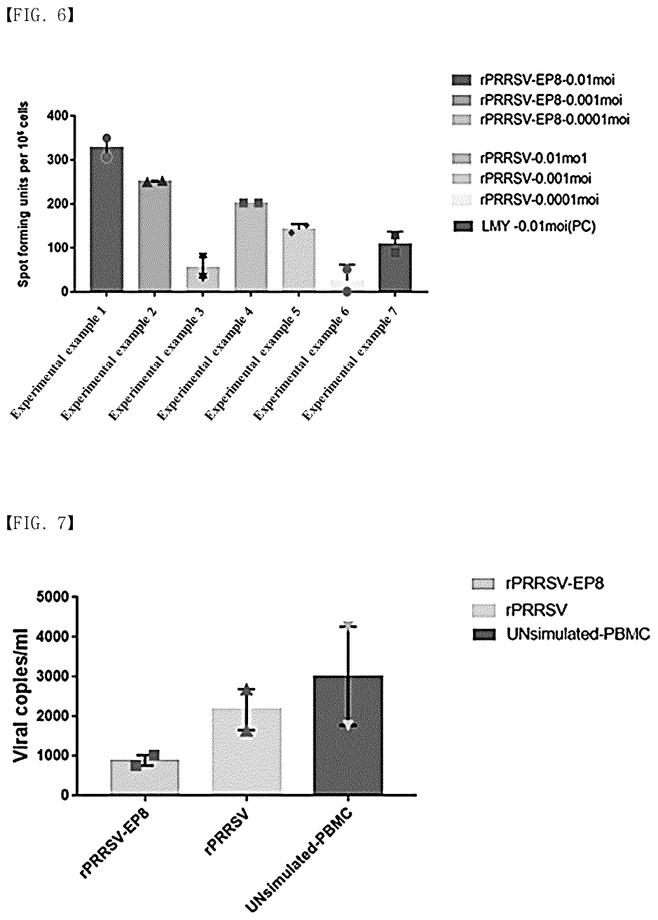

The obtained result was shown in Table 16 and FIG. 6.

TABLE 16

| | | | Spot |
|---|---|---|---|
| | | | forming |
| | | | unit (SFU) |
| | | MOI | per |
| | | (Multiplicity | 5*10⁵ |
| | Vaccine strain | of Infection) | cells |
| Experimental | rPRRSV-EP8 | 0.1 | 327 |
| example 1 | | | |
| Experimental | rPRRSV-EP8 | 0.01 | 251.5 |
| example 2 | | | |
| Experimental | rPRRSV-EP8 | 0.001 | 55 |
| example 3 | | | |
| Experimental | r PRRSV | 0.1 | 202 |
| example 4 | | | |
| Experimental | rPRRSV | 0.01 | 142.5 |
| example 5 | | | |
| Experimental | rPRRSV | 0.001 | 24.5 |
| example 6 | | | |
| Experimental | Inactivated | 0.01 | 108.5 |
| example 7 | LMY | | |

As shown in Table 16 and FIG. 6, at the same MOI, compared to Inactivated LMY obtained by 7 passages of the rPRRSV in which the EP8 gene was not inserted, PRRS LMY parent strain (Accession No. GenBank accession no. DQ473474.1), in MARC cells, the rPRRSV-EP8 expressing EP8 peptide showed a higher IFN-gamma expression level, and this result shows that a more excellent effect of enhancing immunity can be obtained as the vaccine strain expresses the EP8 peptide.

### Example 8. Confirmation of Virus Inhibition Activity of rPRRSV-EP8 Chimeric Virus

The ability of inhibiting the virus by stimulating immunocytes of the (EP8 gene-inserted) rPRRSV-EP8 chimeric virus expressing the EP8 peptide was verified using Virus Suppression Assay (VSA).

Specifically, 3-week-old piglets were infected with PRRS type 2 LMY virus (Accession No. GenBank accession no. DQ473474.1) and then porcine PBMCs (T cells) were isolated and frozen at 5 weeks after the infection. After dissolving the PBMCs, they were released to RPMI-1640 (Life Technologies, Carlsbad, CA, USA) medium in which 10% FBS (Fetal Bovine Serum) was added, and they were added to each well in an amount of 1.25×10\hat{ }6 cells, and then Type 2 PRRS chimeric virus expressing EP8 peptide inactivated with heat (rPRRSV-EP8; Example 2) or rPRRSV (Example 1-3) was cultured at 1.25*10⁴ TCID₅₀ per well, respectively, by stimulating or without stimulating for 6 days. Cells obtained in this way were used as effector cells.

In addition, target cells (monocyte derived macrophages, MDMs) differentiated from monocytes in PBMCs of an autologous or genotype-matched heterologous. More specifically, the frozen PBMCs were dissolved and then washed with serum-free EMEM (Eagle's Minimum Essential Medium) and cultured in serum-free EMEM at 37° C. for 2 hours, and then the medium and suspension cells were discarded and only adherent cells were cultured in 10% FBS-added RPMI-1640 (Life Technologies, Carlsbad, CA, USA) medium for 6 days, and then rat M-CSF (macrophage-colony stimulating factor, Biolegend, San Diego, CA, USA) was added to be 5 ng/ml every 2 days, and differentiated for 6 days to prepare VSA target cells (target MDM cells) (2.5×10\hat{ }5 cells/well). After culturing for 6 days, the effector cells activated by the stimuli prepared above and MDM cells and PRRS LMY (0.00005moi (moi based on MDM cells)) were mixed and cultured. After 4 days of the mixed culture, RNA was extracted using the cells and supernatant and the virus proliferation level was confirmed in the target cells using Real time PCR (QuantiTect Probe RT-PCR Kit; Qiagen).

Specifically, RNA 1 ul was extracted from the mixed and cultured sample supernatant 0.1 ml. The extracted RNA 1 ul was added to a mixture in which QuantiTect Probe RT-PCR buffer 5 ul and Taq man probe and 0.3 ul each of 10 pmol of the PRRSV specific primer set in Table 18 below were mixed, and 2.4 ul of DEPC distilled water was added and they were mixed to perform a real time PCR reaction. The primers used in the real-time PCR were shown in Table 18, and the PCR conditions were shown in Table 17.

TABLE 17
PCR reaction solution

| PCR reaction | | | |
|---|---|---|---|
| solution composition | | Amount | |
| Quant iTect Probe | | 5 | ul |
| RT-PCR buffer | | | |
| Enzyme | | 0.5 | ul |
| Forward Primer | | 0.3 | ul |
| Reverse Primer | | 0.3 | ul |
| Probe | | 0.5 | ul |
| Template RNA | | 1 | ul |
| Distilled water | | 2.4 | ul |
| Total volume | | 10 | ul |
| PCR reaction conditions | | | |
| Reverse transcription | 30 min | 50° | C. |
| Initial PCR activation step | 15 min | 95° | C. |
| 3-step cycling | | | |
| Denaturation | 15 sec | 94° | C. |
| Annealing | 1 min | 60° | C. |
| Extension | 1 min | 72° | C. |
| Number of cycles: 40 | | | |
| Final extension | 10 min | 72° | C. |

TABLE 18

| | | SEQ |
|---|---|---|
| | Base sequence | ID |
| Primer | (5′ → 3′ ) | NO: |
| PRRSV | atgatgrgctggctaact | 17 |
| specific | | |
| Primer | | |
| For | | |
| PRRSV | acaccggtcgccctaatt | 18 |
| specific | g | |
| Primer | | |
| Rev | | |
| Taq man | 5FAM-tgtggtgaatggc | 19 |
| Probe | actgattgaca-3BHQ1 | |

The real-time PCR repeated a total of 35 cycle reactions, and Ct values by each sample was calculated according to the threshold value, and those over 35 were excluded. The Ct values were indicated by converting them into genomic copies by substituting them into the standard curve.

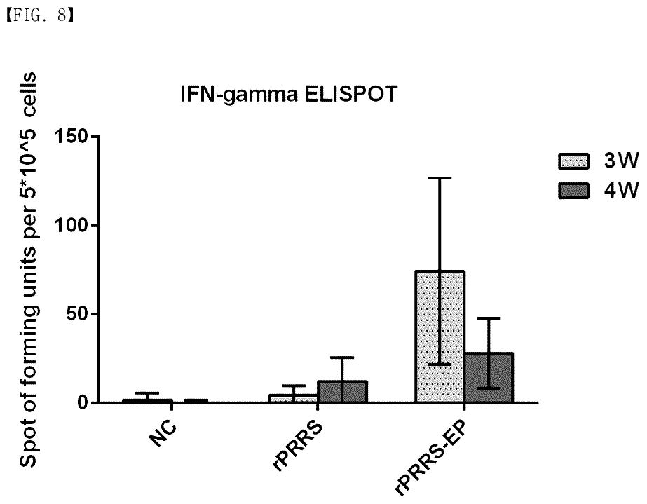

The obtained result was shown in FIG. 7. As shown in FIG. 7, it was confirmed that the virus reduction rate for PBMCs (T cells) without any stimulation was higher in case of PBMC stimulation by the Type 2 PRRS chimeric virus vaccine strain (rPRRSV-EP8) expressing EP8 peptide, compared to the case stimulated with the conventional vaccine strain (rPRRSV) (rPRRSV-EP8: 75%; r PRRSV: 28%). This result shows that the inhibition ability of virus proliferation was significantly increased as the vaccine strain expresses the EP8 peptide.

### Example 9. Immunity Enhancement Confirmation Test (In Vivo)

Using the PBMCs obtained after inoculation with the vaccine composition, cell immunity evaluation was performed by measurement of IFN-gamma.

The IFN-gamma was measured using ELIspot (Enzyme linked immunospot) kit (MABTECH). Specifically, as the experimental groups shown in Table 19 below, the vaccine composition was inoculated into the three groups of 3-week-old pigs by intradermal injection (ID) method at a dose of 5*10⁴ TCID₅₀ (Tissue culture infective dose₅₀).

TABLE 19

| Experi- | | Inoculation | Admini- |
|---|---|---|---|
| mental | | titer | stration |
| group | Vaccine inoculation | (1 dose) | route |
| G1 (n = 4) | Non-inoculation | — | — |
| G2 (n = 6) | rPRRSV(LMY + BP) | 5*10\hat{ }4 TCID₅₀ | ID |
| G3 (n = 6) | rPRRSV-EP7 + rPRRSV-EP8 (1:1) | 5*10\hat{ }4 TCID₅₀ | ID |
| | (rPRRSV-EP, LMY/BP + EP) | | |

Serum and PBMCs were obtained after 21 days (3^{rd} week) and 28 days (4th week) of the vaccine inoculation, and biotin-labeled anti-IFN-gamma primary antibody and streptavidin-HRP-labeled secondary antibody, which were antibodies in the ELISpot kit, were sequentially treated to each well comprising 5*10⁵ cell/mL of the obtained PBMCs, and cultured at 37° C. for 1 hour, and then spot forming unit (SFU) per 5*10⁵ cells was calculated. At this time, the SFU level indicates the IFN-gamma expression level. Mean values of the obtained results were shown in FIG. 8 and Table 20 below.

TABLE 20

| | G1 (n = 4) | G2 (n = 6) | G3 (n = 6) |
|---|---|---|---|
| | (SFU) | (SFU) | (SFU) |
| 3^{rd} week IFN- | 1.625 | 4.167 | 74.167 |
| gamma expression | | | |
| level | | | |
| 4^{th} IFN-gamma | −2.5 | 12.25 | 28 |
| expression level | | | |

As shown in FIG. 8, the amount of IFN-gamma expression was high at the 3^{rd} week in the G3 group (rPRRSV-EP). Through the above experiment, it could be confirmed that stimulation of cellular immunity of peptides occurred by IFN-gamma expression in the G3 group. Through this, in the evaluation of the effect of enhancing cellular immunity, the peptide expressing vaccine inoculation of G3 showed a clear effect of enhancing cellular immunity induction during pig infection. Furthermore, the high level of cellular immunity on day 21 after inoculation suggests the possibility of forming protective immunity in the early stage of vaccine inoculation.

1. A chimeric virus of a PRRSV comprising a polynucleotide consisting of the structure of Structural formula 1 as a genome:
[Structural formula 1]
5′-[X]-[Y]-[Z]-3′
wherein the [X] is a nucleic acid sequence of NSP1 gene ORF1a and ORF1b regions of LMY virus (GenBank accession no. DQ473474.1) or LMY ver2 virus with accession number KCTC 13394BP, and
the [Y] is a nucleic acid sequence encoding a polypeptide of SED ID NO: 3 or 12, and
the [Z] is a nucleic acid sequence of ORF2 to ORF7 regions of BP2017-2 virus with accession number KCTC 13393BP.

2. The chimeric virus according to claim 1, wherein the nucleic acid sequence of NSP1 gene ORF1a and ORF1b regions of the [X] is represented by SEQ ID NO: 1 or SEQ ID NO: 2.

3. The chimeric virus according to claim 1, wherein the [Y] further comprises at least one selected from the group consisting of AscI restriction enzyme recognition site, Kozak sequence, and TRS6 (transcription regulatory sequence6).

4. The chimeric virus according to claim 1, wherein the [Y] is represented by SEQ ID NO: 4, 10, 20 or 21.

5. The chimeric virus according to claim 1, wherein the nucleic acid sequence of ORF2 to ORF7 regions of the [Z] is represented by SEQ ID NO: 5, or further comprises [A] n (the n is the number of base adenine (A), which is an integer selected from 1 to 100) at the 3′ end of SEQ ID NO: 5.

6. The chimeric virus according to claim 1, wherein the polynucleotide is genome of a chimeric virus of a Porcine Reproductive and Respiratory Syndrome Virus (PRRSV), and is DNA or RNA.

7. The chimeric virus according to claim 1, wherein the Structural formula 1 consists of a nucleic acid sequence of SEQ ID NO: 6 or SEQ ID NO: 11.

8. (canceled)

9. The chimeric virus according to claim 1, wherein the chimeric virus has accession number KCTC 14269BP or KCTC 14270BP.

10-17. (canceled)

18. A method for producing a chimeric virus of a Porcine Reproductive and Respiratory Syndrome Virus (PRRSV), comprising
(1) producing a polynucleotide fragment by treating genome of LMY virus (GenBank accession no.DQ473474.1) or LMY ver2 virus of accession number 13394BP with restriction enzymes AscI and PacI;
(2) preparing a polynucleotide fragment comprising a gene encoding a polynucleotide of SEQ ID NO: 3 or 12,
(3) producing a polynucleotide fragment by treating genome of BP2017-2 virus with accession number KCTC 13393BP with restriction enzymes AscI and PacI; and
(4) producing an infectious clone by recombining the polynucleotide fragments obtained in steps (1) to (3) above.

19. The method for producing a chimeric virus according to claim 18, wherein the polynucleotide fragments in the step (2) comprise a nucleic acid sequence of SEQ ID NO: 4, 10, 20 or 21.

20. The method for producing a chimeric virus according to claim 18, wherein the polynucleotide fragments prepared by treating genome of LMY ver2 virus with restriction enzymes AscI and PacI comprise a region encoding NSP1 protein.

21. The method for producing a chimeric virus according to claim 18, wherein the polynucleotide fragments prepared by treating genome of BP2017-2 virus with restriction enzymes AscI and PacI comprises ORF2 to ORF7 regions.

22-27. (canceled)

28. A method for prevention or treatment of porcine reproductive and respiratory syndrome, comprising administering
(1) a chimeric virus of the PRRSV comprising a polynucleotide of Structural formula 2 below as genome or subcultured progeny thereof;
(2) a chimeric virus of the PRRSV comprising a polynucleotide of Structural formula 3 below as genome or subcultured progeny thereof; or
(3) both of them into a pig:
[Structural formula 2]
5′-[X]-[Ya]-[Z]-3′
[Structural formula 3]
5′-[X]-[Yb]-[Z]-3′
wherein the [X] is a nucleic acid sequence of NSP1 gene ORF1a and ORF1b regions of LMY virus (GenBank accession no. DQ473474.1) or LMY ver2 virus with accession number KCTC 13394BP, and
the [Ya] is a nucleic acid sequence comprising an encoding gene of the polypeptide of SED ID NO: 3, and
the [Yb] is a nucleic acid sequence comprising an encoding gene of the polypeptide of SED ID NO: 12, and
the [Z] is a nucleic acid sequence of ORF2 to ORF7 regions of BP2017-2 virus with accession number KCTC 13393BP.

29. The method for prevention or treatment of porcine reproductive and respiratory syndrome according to claim 28,
wherein the [Ya] is represented by SEQ ID NO: 4 or SEQ ID NO: 20, and
the [Yb] is represented by SEQ ID NO: 10 or SEQ ID NO: 21.

30. The method for prevention or treatment of porcine reproductive and respiratory syndrome according to claim 28,
wherein the nucleic acid sequence of NSP1 gene ORF1a and ORF1b regions of the [X] is represented by SEQ ID NO: 1 or SEQ ID NO: 2, and
the nucleic acid sequence of ORF2 to ORF7 regions of the [Z] is represented by SEQ ID NO: 5, or further comprises [A] n (the n is the number of base adenine (A), which is an integer selected from 1 to 100) at the 3′ end of SEQ ID NO: 5.


SEQUENCE LISTING (21 sequences)

SEQ ID NO: 1 - DNA, 1149 bp, Artificial Sequence
  (Synthetic)_LMY_NSP1
atgtctgggatacttgatcggtgcacgtgtacccccaatgccagggtgtttatggcggagggccaagtttactgcacacgatgtctcagtgcacggtctctccttcccctgaatctccaagtttctgagctcggggtgctgggcctattctacaggcccgaagagccactccggtggacgttgccacgtgcattccccactgttgagtgctcccccgccggggcctgctggctttctgcaatctttccaattgcacgaatgaccagtggaaacctaaacttccaacaaagaatggtacgggtcgcagctgagatctacagagttggccagctcacccctgcagttttgaaggctctacaagtttatgagcggggttgccgctggtatcccattgttggacctgtccctggagtggccgttttcgccaactccctgcatgtgagtgatagacctttcccgggagcaactcacgtattaaccaacctgccactcccgcagagacccaagcctgaggacttttgccccttcgagtgtgctatggctgctgtctacgatgttggtcatggcgccgtcatgtatgtggccgatgagagagtctcctgggcccctcgtggcggggatgaagtaagatttgaaactgtcccacaggagctcaagtcggttgcgaaccaactctgcacctccttcccaccccaccacgtagtggacatgtctaagttcgcctttacagcccctgggtgtggtgtttctatgcgggtcgaacgtcaatatggctgtctccccgctgacactgtccccgaaggcaactgctggtggagcttgtttgactcgctcccattggaagtccagggcaaagaaattcgccatgctaaccaatttggctaccagaccaagcatggtgtctctggtaagtacctacagcggaggctgcaaattaatggtctccgagcagtagctgacccaaatggacctttcgtcgtacagtacttctccgtcaaggagagttggatccgccacttgaaactagcggaagaacccagttaccctgggtttgaggacctcctcagaataagggttgagtctaacacgtcaccattggctaacaaggatgaaaaaattttccggtttggcagtcataagtggtacggc

SEQ ID NO: 2 - DNA, 1149 bp, Artificial Sequence
  (Synthetic)_LMY_ver2_NSP1
atgtctgggatacttgatcggtgcacgtgtacccccaatgccagggtgtttatggcggagggccaagtttactgcacacgatgtctcagtgcacggtctctccttcccctgaatctccaagtttctgagctcggggtgctgggcctattctacaggcccgaagagccactccggtggacgttgccacgtgcattccccactgttgagtgctcccccgccggcgcatgctggctttccgctatctttccaatcgcacgaatgaccagtggaaacctaaacttccaacaaagaatggtacgggtcgccgccgaaatatacagagtcgggcaacttacgcccgctgtattgaaggctctgcaagtatacgagcggggttgtaggtggtatcccatcgtaggacctgtccctggagtggccgttttcgccaactccctgcatgtgagtgatagacctttcccgggagcaactcacgtattaaccaacctgccactcccgcagagacccaagcctgaggacttttgccccttcgagtgtgctatggccgccgtatacgacgtcggacatggcgccgttatgtacgttgccgacgagagagtctcctgggcccctcgcggcggcgacgaagttagattcgaaacggtcccacaggagcttaagtcggttgcgaaccaattatgtacgtcgttcccaccccaccacgtagtcgatatgtctaagttcgcctttaccgcccccggttgcggcgtatctatgcgggtcgaacgtcaatacggctgtctccccgccgatacggtccccgaaggcaactgttggtggagcttgttcgattcgctcccactcgaagtgcaaggcaaagagattcgccacgctaaccaattcgggtatcagactaagcatggcgtatccggtaagtacctacagcgtaggctgcaaatcaacggtctccgcgcagtcgctgaccctaacggacctttcgtcgtacagtacttctccgtcaaggagagttggatccgccacttgaaactggccgaagaacctagttaccccgggttcgaggacctcctccgcataagggttgagtctaatacgtcaccattggctaacaaggacgaaaaaattttccggtttggcagtcataagtggtacggc

SEQ ID NO: 3 - protein, 26 aa, Artificial Sequence
  (Synthetic)_T cell epitope_EP7_Amino_Acid
LLAFSITYTPVMIYALKVSRGRLLGL

SEQ ID NO: 4 - DNA, 78 bp, Artificial Sequence
  (Synthetic)_T cell epitope_EP7
ctgctagctttcagtatcacatatactccagttatgatatacgcacttaaggtctctcgtgggcgtctcctagggctc

SEQ ID NO: 5 - DNA, 3188 bp, Artificial Sequence
  (Synthetic)_BP2017-2_ORF2-ORF7
atgaaatggggtccatgcaaagccttttttacaaaattggccaactttttgtggatgctttcacggagttcttggtgtccattgttgatatcattatatttttggccattttgtttggcttcaccatcgccggttggctggtggtcttttgcatcagattggtttgctccgcgatactccgtacgcgccctgccattcactctgagcaattacagaagatcttatgaggcctttctttcccagtgccaagtggacattcccacctggggaactaaacatcctttggggatgctttggcaccataaggtgtcaaccctgattgatgaaatggtgtcgcgtcgaatgtaccgcatcatggaaaaagcagggcaggctgcctggaaacaggtggtgagcgaggctacgctgtctcgcattagtagtttggatgtggtggctcattttcagcatctagccgccattgaagccgagacctgtaaatatttggcctcccggctgcccatgctacacaacctgcgcatgacaggttcaaatgtaaccatagtgtataatagcactttgaatcaggtgtttgctatttttccaacccctggttcccggccaaagcttcatgattttcagcaatggttaatagctgtacattcctccatattttcctctgttgcagcttcttgtactctttttgttgtgctgtggttgcgggttccaatactacgtactgtttttggtttccgctggttaggggcaatttttctttcgaactcacagtgaattacacggtgtgtccaccttgcctcacccggcaagcagccacagagatctacgaacccggtaggtctctttggtgcaggatagggtatgaccgatgtgaggaggatgatcatgacgagctagggtttatggtaccgcctggcctctccagcgaaggccacttgactagtgtttacgcctggttggcgttcttgtccttcagctacacggcccagttccatcccgagatattcgggatagggaatgtgagtcgagtttatgttgacatcaaacatcaactcatctgcgccgaacatgacgggcagaacaccaccttgcctcgtcatgacaacatttcagccgtgtttcagacctattaccaacatcaagtcgacggcggcaattggtttcacctagaatggcttcgtcccttcttttcctcgtggttggttttaaatgtctcttggtttctcaggcgttcgcctgcaaaccatgtttcagttcgagtcttgcagatattaagaccaacaccaccgcagcggcaagctttgctgtcctccaagacatcagttgccttaggcatcgcgactcggcctctgaggcgattcgcaaaatccctcagtgccgtacggcgatagggacacccgtgtatgttaccatcacagccaatgtgacagatgagaattatttacattcttctgatctcctcatgctttcttcttgccttttctatgcttctgagatgagtgaaaagggatttaaggtggtatttggcaatgtgtcaggcatcgtggctgtgtgtgtcaattttaccagctacgtccaacatgtcaaggagtttacccaacgctccctggtggtcgaccatgtgcggttgctccatttcatgacacctgagaccatgaggtgggcaactgttttagcctgtctttttgccattctgttggcaatttgaatgtttaagtatgttggagaaatgcttgaccgcgggctgttgctcgcaattgctttctttgtggtgtatcgtgccgttctgttttgctgtgctcgccaacgccagcaacgacagcagctcccatctacagctgatttacaacttgacgctatgtgagctgaatggcacagattggctagctaacaaatttgattgggcagtggagagttttgtcatctttcccgttttgactcacattgtctcctatggtgccctcactaccagccatttccttgacacagtcgctttagtcactgtgtctaccgccgggtttgttcacgggcggtatgtcctaagtagcatctacgcggtctgtgccctggctgcgttgacttgcttcgtcattaggtttgcaaagaattgcatgtcctggcgctacgcgtgtaccagatataccaactttcttctggacactaagggcggactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccagaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatga

SEQ ID NO: 6 - DNA, 15581 bp, Artificial Sequence
  (Synthetic)_PRRSV-EP7 chimera full genome
atgacgtataggtgttggctctatgccttgacatttgtattgtcaggagctgtgaccattggcacagtccaaaacttgctgcgcagaaacacccttctgcgacagcctccttcaggggagcttggggtctatccctagcaccttgcttctggagttgcactgctttacggtctctccacccctttaaccatgtctgggatacttgatcggtgcacgtgtacccccaatgccagggtgtttatggcggagggccaagtttactgcacacgatgtctcagtgcacggtctctccttcccctgaatctccaagtttctgagctcggggtgctgggcctattctacaggcccgaagagccactccggtggacgttgccacgtgcattccccactgttgagtgctcccccgccggcgcatgctggctttccgctatctttccaatcgcacgaatgaccagtggaaacctaaacttccaacaaagaatggtacgggtcgccgccgaaatatacagagtcgggcaacttacgcccgctgtattgaaggctctgcaagtatacgagcggggttgtaggtggtatcccatcgtaggacctgtccctggagtggccgttttcgccaactccctgcatgtgagtgatagacctttcccgggagcaactcacgtattaaccaacctgccactcccgcagagacccaagcctgaggacttttgccccttcgagtgtgctatggccgccgtatacgacgtcggacatggcgccgttatgtacgttgccgacgagagagtctcctgggcccctcgcggcggcgacgaagttagattcgaaacggtcccacaggagcttaagtcggttgcgaaccaattatgtacgtcgttcccaccccaccacgtagtcgatatgtctaagttcgcctttaccgcccccggttgcggcgtatctatgcgggtcgaacgtcaatacggctgtctccccgccgatacggtccccgaaggcaactgttggtggagcttgttcgattcgctcccactcgaagtgcaaggcaaagagattcgccacgctaaccaattcgggtatcagactaagcatggcgtatccggtaagtacctacagcgtaggctgcaaatcaacggtctccgcgcagtcgctgaccctaacggacctttcgtcgtacagtacttctccgtcaaggagagttggatccgccacttgaaactggccgaagaacctagttaccccgggttcgaggacctcctccgcataagggttgagtctaatacgtcaccattggctaacaaggacgaaaaaattttccggtttggcagtcataagtggtacggcgctggtaagagagcaaggaaagcacgctctcgtgcgactaatacagtcgctgaccgcgctttgtccgttcgtgaaatctggcaggccaaggagcatgaggttaccggcgccaataaggctgagcacctcaaacactactccccacctgccgaagggaattgtggttggcactgcatttccgccatcgtcaaccggatggttaattccaaatttgaaaccacccttcccgaaagagtgagacctccagatgactgggctaccgacgaggatcttgcgaatgccatccaaatcctcagactccctgcggccttagacaggaacggtgcttgtgctagcgccaagtacgtacttaagctggaaggtgagcattggactgtcactgtgacccctgggatgtccccttctttgctccctcttgaatgtgttcagggctgttgtgggcacaagggcggtcttggttccccagacgcagtcgaggtctccggatttgaccctgcctgccttgaccgtctggctgaggtgatgcacctgcctagcagtgctatcccagccgctctggccgaaatgtctggtgattccgatcgttcggcttctccggtcaccaccgtgtggactgtttcgcagttctttgcccgtcacagcggaggaaatcaccctgaccaagtgcgcttagggaaaatcatcagcctttgtcaggtgattgaggactgctgctgttcccagaacaaaaccaaccgggtcaccccggaggaggttgcagcaaagattgacctgtacctccgcggtgcaacaaatcttgaagaatgcttggccaggctcgagaaagcgcgcccgccacgggtaatcgacaccttctttgattgggacgttgtgctccctggggttgaggcagcaacccaaacgaccaagctgccccaggtcaaccagtgtcgtgctctggtccctgttgtgactcaaaagtccttggacaacaactcggtccccctgaccgccttttcactggctaactactactaccgcgcgcaaggtgacgaagtttgtcaccgtgaaagacttaatgccgtgctttccaagctggaagaggttgtccgagaagagtacgggctcatgcctaccgggcctggcccacggcccacattgccacgcgggctcgacgaactcaaagaccagatggaggtggacttgctgaaactggctaacgcccaggcgacttctgacatgatggcttgggcagtcgagcaggttgacctaaaaacttgggtcaagaactacccgcggtggatgccaccacctcctccgccaaaagttcggcctcgaaaaacgaagcctgtcaagagcttgccagagggaaagcccgcccccgcccctcgcaggagggttgggactggttgtggcagcccgacttcattgggcgatgatgttcttaacagttgggaagatttggctgttggcagcccctcgggtctcccgaccccccctgagccggcaacaccttcgagtgagccagtggttgtgtcagcaccgcagtgcatcttcagaccggcgacgcccttgagtgagccgaccccaattcccgcaccccgtgggactgcgtctcgaccagtgacacctttgagcgagccgatccctgtgcctgcaccgcggcgtaaatttcagcaggcgagaaagttgagttcggcggtggtaattccgccgtaccaggacgagcccctggatttgtccgcttcctcgctgactgaatatgaagcccttcctctagcgccgccgcaaagcgagggcgctctaagagtggaggggcatgaagttgaggaagccctgagtgaaatctcagacatgtcgggtggcactaaacctgcgcccgtatcatcaagcagctccttgtccagcgtgagaatcacacgcccaaaacactcagctcaagccatcatcgactcggtcgggccctgcagtgggcatctccaagaggtgaaggagatatgccttagtgtcatgcgcgaggcatgtgatgcgactaagctcaatgaccccgctacgcaggaatggctctctcgcatgtgggatcgggtggacatgctgacttggcgcaacacgtctgcttaccagacgcttagcaccttaggtggctggtcaaggttcctcccaaaattgatacttgagacaccgccgccctacccgtgtgagtttgtgatgatgccttacacgcctgcaccttccgtgggtgcggagagcgacctcaccattggctcagttgctactgaggatgttccacgcatccttgagaacatggagaatgtcggcgagatgactaaccagggacccttggccttctccgaggataaaccggtggatgaccaaccttccgaagacctccggacatcgtcgcaaggacctgacgggagcacatcagctccgtccgcagatacagctggcgccggctcatccaccgatttgccgtcttcaggcggcgtggatgcggacggagggggaccgcttcgggcggtaaaaagaaaaaccgaacggctctttgaccaactgagtcgtcaggtttttgacctcgtctcccatctccctattttcttctcacgcctcttcaaccctggcggtggttactctccgggtgattggggttttgcagcttttactctattgtgcctctttttatgttacagttacccagcatttggcatcgttcccctcttgggtgtattttctgggtcttctcggcgcgtgcgaatgggggtttttggctgctggttggcttttgctgttggtctgttcaagcctgtgtccgacccagtcggcgctgcttgtgagtttgactcgccagaatgcagaaacatccttcattcttttgagcttctcaaaccttgggaccctgttcgcagccttgttgtgggccccgtcggtctcggttttgccattcttggcaggctactgggcggggcacgcagcatctggcactttttgcttaggcttggcattgttgcagactgtgtcttggctggagcttatgtgctttctcaaggtaggtgcaaaaagtgttggggatcttgtataagaaccgctcctaatgaggtcgctttcaacgtgtttccttttacgcgtgcaaccaggtcgtcacttgttgacctttgcgatcggttttgtgcgccgagaggcatggaccccatttttctcgccactgggtggcgtggatgctgggccggtcggagccccattgagcaaccctctgaaaaacccatcgcgtttgcccaattggatgaaaagaagattacggctaggactgtggtcgcccagccttatgatcccaaccaagccgtaaagtgcctgcgggtattgcaggcgggaggggcgatggtggctgaggcggtcccaaaagtggtcagggtttccgctgttccatttcgagcccccttttttcctaccggagtgaaggttgaccctgaatgtagagtagtggttgaccctgacactttcactgcagctctccggtctggttactccaccacaaacctcgtcctcggtgtgggggactttgcccagctgaatggattaaaaatcaggcaaatttctaagccttcaggggggggcccacatctcatggctgccctgcatgttgcctgctcgatgattttgcacatgtttgctgggatttatgtgactgcggtgggttcttgcggcaccggcaccaacgatccgtggtgcgcaaacccatttgccgtccctggctacggacctggctccctctgcacgtccagattgtgtatttcccagcacggccttaccctgcccttgacagcacttgtggcgggattcggcatccaggaaattgccttagtcgctttgatctttgtttccatcggaggcattgctcataggttgagttgtaaagctgacatgctgtgtgttttgcttgcaattgccagctatgtttgggtacctcttacctggttactttgtgtttttccttgctggttgcgctggttttctttgcatccccttaccgtcctatggttggtgtttttcttgatttctgtgaatatgccttcaggaatcttggccatggtgttgttagtttctctttggcttcttggtcgttatactaatgtcgccggtcttgtcactccctacgacattcatcattacaccagtggcccccgcggtgttgccgcattggctactgcaccagatggaacctacttagccgctgttcgccgcgctgcgttgactggccgcaccatgttgttcaccccgtcccagcttgggtctcttcttgagggcgctttcagaactcgaaagccctcactgaacaccgtcaatgtggtcgggtcctccatgggctctggcggggtgtttaccattgacgggaaagtcaggtgcgtgactgccacacatgtccttacgggtaactcagccagggtttccggaaccggcttcaatcaaatgcttgactttgacgtaaaaggggatttcgctatagccgattgcccgaattggcaaggggctgcccccaaaacccaattctgcgaggatggatggactggccgtgcctattggctaacatcctctggcgtcgaacccggcgtcattgggaaaggattcgccttctgcttcaccgcgtgcggcgattccgggtccccagtgatcaccgaggccggtgagcttgtcggtgttcacacgggatcgaataaacaaggggggggtattgtcacacgcccctcaggccagttttgtaatgtggcacccatcaagctgagcgaattaagtgaattctttgctgggcctaaggtcccgctcggtgatgtgaaggttggcagccacataatcaaagacacaagcgaggtgccttcagatctttgtgccttgcttgctgccaaacctgaactggaaggaggtctctccaccgtccaacttctgtgtgtgtttttccttctgtggagaatgatgggacatgcctggacgcccttggttgctgtgggtttctttattttgaatgagattctcccggccgtcctggtccggagtattttctcctttggaatgtttgtgctatcctggctcacgccatggtctgcgcaagttctgatgatcaggctcctgacagcagctcttaacaggaatagatgttcacttgcctttttcagcctcggtgcagtgaccggttttgttgcagatcttgcggctactcaggggcatccgttgcaggcagtgatgaatttgagcacctatgcattcctgcctcgggtaatggttgtgacttcaccagtcccagtgatcgcgtgtggtgttgtgcacctactcgccatcattctgtacctgtttaaataccgtagcctgcaccatatccttgttggcgatggagtgttctcttcggccttcttcctgcgatattttgccgaaggaaagttgagggaaggggtgtcacaatcctgcggaatgaatcatgagtctctgactggtgccctcgctatgagactcaatgacgaggacttggatttcctcatgaaatggactgattttaagtgcttcgtttctgcgtccaacatgaggaatgcagcgggtcaatttatcgaggctgcatatgctaaagcacttagagttgaactggcccagttggtgcaggttgataaggttcgaggtactatggccaaacttgaagcttttgctgatactgtggcacctcaactctcgcccggtgatattgttgtcgctctcggccacacgcctgttggcagtatcttcgacttaaaggttggtagcaccaagcataccctccaagccattgagaccagggttcttgctgggtccaaaatgaccgtggcgcgcgtcgtcgacccgacccccacgcccccacccgcgcccgtgcccatccccctcccaccgaaagttctggagaatggccccaacgcttggggggatgaggaccgtttgaataagaagaaaagacgcaggatggaagccctcggcatctatgttatgggcgggaaaaagtaccagaaattttgggacaagaactccggtgatgtgttttatgaggaggtccataacaacacagatgagtgggagtgtctcacagttggcgaccctgccgactttgaccctgagaagggaaccctgtgtggacatgtcaccattgacaacaaggcttaccatgtttacacctcctcatctggtaagaagttcttggttcccgtcaacccagagaacggaagagttcaatgggaagctgcaaagctttccgtggagcaggccctaggcatgatgaatgtcgacggcgaactgactgccaaggaactggagaaactgaaaagaataattgacaaactccagggcctgactaaggagcagtgtttaaactgctagccgccagcgacttgacccgctgtggtcgcggcggcttggttgttactgagacagcggtgaaaatagtcaaatttcacaatcggactttcactctggggcctgtgaatttaaaagtggccagtgaggttgagctaaaagatgcggttgagcacaaccaacacccggttgcgaggccgatcgatggtggagttgtgctcctgcgttccgcggttccctcgcttatagacgtcttgatctccggtgctgacacatctcccaagttacttgcccttcacgggccgggaaacactgggatcgatggcacgctctgggatttcgagtccgaagccactaaagaggaagtcgcactcagtgcgcaaataatacaggcttgtgacattaggcgtggcgacgctcctgaaattggtctcccttataagctgtaccctgttaggggcaaccctgagcgggtgaaaggagttctgcagaatacaaggtttggagacataccctacaaaacccctagtgacaccgggagcccagtgcacgcggctgcctgccttacgcccaacgccactcctgtgactgatgggcgttccgtcttggctacaaccatgccccccgggtttgagttatatgtaccgaccataccagcgtctgtccttgattaccttgactctaggcctgactgccctaaacagctgacagagcacggctgcgaagacgccgcactgaaagacctctctaaatatgacttatctacccaaggctttgtcttacctggggttcttcgccttgtgcggaaatacctgtttgcccatgtaggtaaatgtccacccgttcatcggccttctatttaccccgctaagaattctatggctggaataaatgggaacaggttcccaaccaaagacatccagagcgttcctgaaatcgacgttctgtgcgcacaggctgtgcgggaaaactggcaaactgtcaccccttgtactcttaagaaacagtattgcgggaagaagaagaccaggaccatactcggcaccaataacttcattgcactggcccaccgagcagtgttgagtggtgtcacccagggcttcatgaaaaaggcgttcaactcgcccatcgccctcggaaagaacaagttcaaggagctacagactccggtcctgggcaggtgccttgaagctgacctcgcatcctgtgatcgatccacgcctgcaattgttcgctggtttgctgccaaccttctttatgaacttgcctgtgctgaagagtatctaccgtcgtatgtgctgaactgctgccacgacttactagtcacgcagtccggcgcagtgactaagagaggtggcctgtcgtctggcgacccgatcacctctgtgtctaacaccatttacagtttggtgatctatgcacagcacatggtgcttagttactttaaaagtggtcacccccatggccttctgttcttacaagaccagctaaagtttgaggacatgctcaaggttcaacccctgatcgtctattcggacgatctcgtgctgtatgccgagtctcccaccatgccaaattatcactggtgggttgagcatctgaatttgatgctggggtttcagacggacccaaaaaaaacagcaataacagattcgccatcatttctgggctgtagaataatgaatgggcgccagctagtccccaaccgtgacaggatcctcgcggccctcgcctatcacatgaaggcaagtaatgtttctgaatactatgcctcagcggctgcaatactcatggacagctgtgcttgtttggagtatgatcctgaatggtttgaagaacttgtagttggaatagcgcagtgcgcccgcaaggatggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatctaccacacccacttccaccagcattgtccagtcacaatctggtgtggccatccagcgggttctggttcttgtagtgagtgcaaatcccctgtagggaaaggcacaaaccctttagacgaggtgctggaacaagtcccgtataagcccccacggaccgttatcatgcatgtagagcagggtctcaccccccttgatccaggtagataccaaactcgccgcggattagtttccgttaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgcagggaaaacatactggctccttcaacaggtccaggacggtgatgttatttacacaccaactcaccagaccatgcttgacatgatcagggctttggggacgtgccggttcaacgtcccggcaggcacaacgctgcaattccctgtcccctcccgcaccggtccgtgggttcgcatcctggccggcggttggtgtcctggcaagaattccttcctagatgaagcagcgtattgcaaccaccttgatgttttgaggcttcttagcaaaactaccctcacctgtctaggagacttcaagcaactccacccagtgggttttgattctcattgttatgtttttgacatcatgcctcaaactcaactgaagaccatctggaggttcggacagaatatctgtgatgccatccagccagattacagggacaagctcatgtccatggtcaacacaacccgtgtgacctacgtggaaagacctgtcaggtatgggcaggtcctcaccccctaccacagggaccgagaggacgacgccatcactattgactccagtcaaggcgccacattcgatgtggtcacattgcatttgcccactaaagattcactcaacaggcaaagagcccttgttgccatcaccagggctagacacgctatctttgtgtatgacccacacaggcagctgcagagcttgtttgatcttcctgcaaaaggcacacccgtcaaccttgcagtgcaccgcgacgggcagctgatcgtacttgatagaaataacaaagaatgcacggttgctcaggctctaggcaacggggataaattcagggccacagacaagcgtgttgcagattcactccgcgccatttgtgctgatctagaagggtcgagttctccgctccccaaggtcgcacacaacttggggttttatttctcacctgacttaacacaatttgccaaactcccagtggaacttgcacctcattggcccgtggtgacaacccagaacaatgaaaagtggccagatcggctggtcgccagccttcgccctatccataaatacagccgcgcgtgcatcggtgccggctatatggtgggcccttcggtgtttctaggcactcctggggtcgtatcatactttcttacaaaatttgttaaaggcgaggctcaaatgcttccagaaacagtcttcagcaccggccgaattgaggtggattgccgggaatatcttgacgatcgggagcgggaagttgctgcatccctcccacacgcctttattggcgacgtcaaaggcaccaccgttggcggatgtcatcatgtcacctccaggtaccttcctcgcttccttcccaaagagtcagttgcggtagtcggggtctcaagccccgggaaagccgcgaaggcaatgtgcacactgacagatgtgtacctcccagatcttgaagcctatctccacccggagacccagtccaggtgctggaaaatgatgttggacttcaaagaagttcgactgatggtctggaaagacaaaacggcctatttccaacttgagggtcgttatttcacctggtatcagctcgctagttatgcctcgtacgtccgtgttcctgtcaactccacggtgtacttggacccctgcatgggcccagcccattgcaacagacgagttgtcgggtccactcagtggggagctgacctcgcaatcaccccctatgattatggcgccaagatcattttgtctagcgcatatcacggtgaaatgccccctgggtacaagattctggcgtgtgcggagttctcattggatgacccggtcagatacaaacacacctggggatttgagtcggatacagcgtacctgtacgagttcactggaaatggtgaggattgggaagattataatgatgcgtttcgtgcgcgccagaaagggaaaatttataaggccactgccaccagcttgaagttttatttccctccgggccctgtcgttgaaccaactttgggcctgaactgaaggcgcgccgccaccatgctgctagctttcagtatcacatatactccagttatgatatacgcacttaaggtctctcgtgggcgtctcctagggctctaggttccgtggcaacccctataaccagagtttcagcggaacaggcgcgccatgaaatggggtccatgcaaagccttttttacaaaattggccaactttttgtggatgctttcacggagttcttggtgtccattgttgatatcattatatttttggccattttgtttggcttcaccatcgccggttggctggtggtcttttgcatcagattggtttgctccgcgatactccgtacgcgccctgccattcactctgagcaattacagaagatcttatgaggcctttctttcccagtgccaagtggacattcccacctggggaactaaacatcctttggggatgctttggcaccataaggtgtcaaccctgattgatgaaatggtgtcgcgtcgaatgtaccgcatcatggaaaaagcagggcaggctgcctggaaacaggtggtgagcgaggctacgctgtctcgcattagtagtttggatgtggtggctcattttcagcatctagccgccattgaagccgagacctgtaaatatttggcctcccggctgcccatgctacacaacctgcgcatgacaggttcaaatgtaaccatagtgtataatagcactttgaatcaggtgtttgctatttttccaacccctggttcccggccaaagcttcatgattttcagcaatggttaatagctgtacattcctccatattttcctctgttgcagcttcttgtactctttttgttgtgctgtggttgcgggttccaatactacgtactgtttttggtttccgctggttaggggcaatttttctttcgaactcacagtgaattacacggtgtgtccaccttgcctcacccggcaagcagccacagagatctacgaacccggtaggtctctttggtgcaggatagggtatgaccgatgtgaggaggatgatcatgacgagctagggtttatggtaccgcctggcctctccagcgaaggccacttgactagtgtttacgcctggttggcgttcttgtccttcagctacacggcccagttccatcccgagatattcgggatagggaatgtgagtcgagtttatgttgacatcaaacatcaactcatctgcgccgaacatgacgggcagaacaccaccttgcctcgtcatgacaacatttcagccgtgtttcagacctattaccaacatcaagtcgacggcggcaattggtttcacctagaatggcttcgtcccttcttttcctcgtggttggttttaaatgtctcttggtttctcaggcgttcgcctgcaaaccatgtttcagttcgagtcttgcagatattaagaccaacaccaccgcagcggcaagctttgctgtcctccaagacatcagttgccttaggcatcgcgactcggcctctgaggcgattcgcaaaatccctcagtgccgtacggcgatagggacacccgtgtatgttaccatcacagccaatgtgacagatgagaattatttacattcttctgatctcctcatgctttcttcttgccttttctatgcttctgagatgagtgaaaagggatttaaggtggtatttggcaatgtgtcaggcatcgtggctgtgtgtgtcaattttaccagctacgtccaacatgtcaaggagtttacccaacgctccctggtggtcgaccatgtgcggttgctccatttcatgacacctgagaccatgaggtgggcaactgttttagcctgtctttttgccattctgttggcaatttgaatgtttaagtatgttggagaaatgcttgaccgcgggctgttgctcgcaattgctttctttgtggtgtatcgtgccgttctgttttgctgtgctcgccaacgccagcaacgacagcagctcccatctacagctgatttacaacttgacgctatgtgagctgaatggcacagattggctagctaacaaatttgattgggcagtggagagttttgtcatctttcccgttttgactcacattgtctcctatggtgccctcactaccagccatttccttgacacagtcgctttagtcactgtgtctaccgccgggtttgttcacgggcggtatgtcctaagtagcatctacgcggtctgtgccctggctgcgttgacttgcttcgtcattaggtttgcaaagaattgcatgtcctggcgctacgcgtgtaccagatataccaactttcttctggacactaagggcggactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccagaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggtgagatttaattggcgagaaccatgcggccgaaattaaaaaaaaaaaaaaaaaaaaaaaa

SEQ ID NO: 7 - DNA, 24 bp, Artificial Sequence
  (Synthetic)_[A]n sequence
aaaaaaaaaaaaaaaaaaaaaaaa

SEQ ID NO: 8 - DNA, 8 bp, Artificial Sequence
  (Synthetic)_ORF2-7_AscI
ggcgcgcc

SEQ ID NO: 9 - DNA, 8 bp, Artificial Sequence
  (Synthetic)_ORF2-7_PacI
ttaattaa

SEQ ID NO: 10 - DNA, 114 bp, Artificial Sequence
  (Synthetic)_T cell epitope_EP8
ctgtggggcgtatattccgcaatcgaaacgtggaagttcataacgagtcgctgccgtctctgcttattagggcgaaagtatatactcgctcctgctcaccacgtggagtcggct

SEQ ID NO: 11 - DNA, 18328 bp, Artificial Sequence
  (Synthetic)_PRRSV-EP8 chimera full genome
gaattcagccagcaagacagcgatatcacctgtaagtcggacgaattcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagcatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacctgtaagtcggacgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacgtgagcgatcgcatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccacgtcatctgatgaggccgaaaggccgaaacggtgaaagccgtaatgacgtataggtgttggctctatgccttgacatttgtattgtcaggagctgtgaccattggcacagtccaaaacttgctgcgcagaaacacccttctgcgacagcctccttcaggggagcttggggtctatccctagcaccttgcttctggagttgcactgctttacggtctctccacccctttaaccatgtctgggatacttgatcggtgcacgtgtacccccaatgccagggtgtttatggcggagggccaagtttactgcacacgatgtctcagtgcacggtctctccttcccctgaatctccaagtttctgagctcggggtgctgggcctattctacaggcccgaagagccactccggtggacgttgccacgtgcattccccactgttgagtgctcccccgccggcgcatgctggctttccgctatctttccaatcgcacgaatgaccagtggaaacctaaacttccaacaaagaatggtacgggtcgccgccgaaatatacagagtcgggcaacttacgcccgctgtattgaaggctctgcaagtatacgagcggggttgtaggtggtatcccatcgtaggacctgtccctggagtggccgttttcgccaactccctgcatgtgagtgatagacctttcccgggagcaactcacgtattaaccaacctgccactcccgcagagacccaagcctgaggacttttgccccttcgagtgtgctatggccgccgtatacgacgtcggacatggcgccgttatgtacgttgccgacgagagagtctcctgggcccctcgcggcggcgacgaagttagattcgaaacggtcccacaggagcttaagtcggttgcgaaccaattatgtacgtcgttcccaccccaccacgtagtcgatatgtctaagttcgcctttaccgcccccggttgcggcgtatctatgcgggtcgaacgtcaatacggctgtctccccgccgatacggtccccgaaggcaactgttggtggagcttgttcgattcgctcccactcgaagtgcaaggcaaagagattcgccacgctaaccaattcgggtatcagactaagcatggcgtatccggtaagtacctacagcgtaggctgcaaatcaacggtctccgcgcagtcgctgaccctaacggacctttcgtcgtacagtacttctccgtcaaggagagttggatccgccacttgaaactggccgaagaacctagttaccccgggttcgaggacctcctccgcataagggttgagtctaatacgtcaccattggctaacaaggacgaaaaaattttccggtttggcagtcataagtggtacggcgctggtaagagagcaaggaaagcacgctctcgtgcgactaatacagtcgctgaccgcgctttgtccgttcgtgaaatctggcaggccaaggagcatgaggttaccggcgccaataaggctgagcacctcaaacactactccccacctgccgaagggaattgtggttggcactgcatttccgccatcgtcaaccggatggttaattccaaatttgaaaccacccttcccgaaagagtgagacctccagatgactgggctaccgacgaggatcttgcgaatgccatccaaatcctcagactccctgcggccttagacaggaacggtgcttgtgctagcgccaagtacgtacttaagctggaaggtgagcattggactgtcactgtgacccctgggatgtccccttctttgctccctcttgaatgtgttcagggctgttgtgggcacaagggcggtcttggttccccagacgcagtcgaggtctccggatttgaccctgcctgccttgaccgtctggctgaggtgatgcacctgcctagcagtgctatcccagccgctctggccgaaatgtctggtgattccgatcgttcggcttctccggtcaccaccgtgtggactgtttcgcagttctttgcccgtcacagcggaggaaatcaccctgaccaagtgcgcttagggaaaatcatcagcctttgtcaggtgattgaggactgctgctgttcccagaacaaaaccaaccgggtcaccccggaggaggttgcagcaaagattgacctgtacctccgcggtgcaacaaatcttgaagaatgcttggccaggctcgagaaagcgcgcccgccacgggtaatcgacaccttctttgattgggacgttgtgctccctggggttgaggcagcaacccaaacgaccaagctgccccaggtcaaccagtgtcgtgctctggtccctgttgtgactcaaaagtccttggacaacaactcggtccccctgaccgccttttcactggctaactactactaccgcgcgcaaggtgacgaagtttgtcaccgtgaaagacttaatgccgtgctttccaagctggaagaggttgtccgagaagagtacgggctcatgcctaccgggcctggcccacggcccacattgccacgcgggctcgacgaactcaaagaccagatggaggtggacttgctgaaactggctaacgcccaggcgacttctgacatgatggcttgggcagtcgagcaggttgacctaaaaacttgggtcaagaactacccgcggtggatgccaccacctcctccgccaaaagttcggcctcgaaaaacgaagcctgtcaagagcttgccagagggaaagcccgcccccgcccctcgcaggagggttgggactggttgtggcagcccgacttcattgggcgatgatgttcttaacagttgggaagatttggctgttggcagcccctcgggtctcccgaccccccctgagccggcaacaccttcgagtgagccagtggttgtgtcagcaccgcagtgcatcttcagaccggcgacgcccttgagtgagccgaccccaattcccgcaccccgtgggactgcgtctcgaccagtgacacctttgagcgagccgatccctgtgcctgcaccgcggcgtaaatttcagcaggcgagaaagttgagttcggcggtggtaattccgccgtaccaggacgagcccctggatttgtccgcttcctcgctgactgaatatgaagcccttcctctagcgccgccgcaaagcgagggcgctctaagagtggaggggcatgaagttgaggaagccctgagtgaaatctcagacatgtcgggtggcactaaacctgcgcccgtatcatcaagcagctccttgtccagcgtgagaatcacacgcccaaaacactcagctcaagccatcatcgactcggtcgggccctgcagtgggcatctccaagaggtgaaggagatatgccttagtgtcatgcgcgaggcatgtgatgcgactaagctcaatgaccccgctacgcaggaatggctctctcgcatgtgggatcgggtggacatgctgacttggcgcaacacgtctgcttaccagacgcttagcaccttaggtggctggtcaaggttcctcccaaaattgatacttgagacaccgccgccctacccgtgtgagtttgtgatgatgccttacacgcctgcaccttccgtgggtgcggagagcgacctcaccattggctcagttgctactgaggatgttccacgcatccttgagaacatggagaatgtcggcgagatgactaaccagggacccttggccttctccgaggataaaccggtggatgaccaaccttccgaagacctccggacatcgtcgcaaggacctgacgggagcacatcagctccgtccgcagatacagctggcgccggctcatccaccgatttgccgtcttcaggcggcgtggatgcggacggagggggaccgcttcgggcggtaaaaagaaaaaccgaacggctctttgaccaactgagtcgtcaggtttttgacctcgtctcccatctccctattttcttctcacgcctcttcaaccctggcggtggttactctccgggtgattggggttttgcagcttttactctattgtgcctctttttatgttacagttacccagcatttggcatcgttcccctcttgggtgtattttctgggtcttctcggcgcgtgcgaatgggggtttttggctgctggttggcttttgctgttggtctgttcaagcctgtgtccgacccagtcggcgctgcttgtgagtttgactcgccagaatgcagaaacatccttcattcttttgagcttctcaaaccttgggaccctgttcgcagccttgttgtgggccccgtcggtctcggttttgccattcttggcaggctactgggcggggcacgcagcatctggcactttttgcttaggcttggcattgttgcagactgtgtcttggctggagcttatgtgctttctcaaggtaggtgcaaaaagtgttggggatcttgtataagaaccgctcctaatgaggtcgctttcaacgtgtttccttttacgcgtgcaaccaggtcgtcacttgttgacctttgcgatcggttttgtgcgccgagaggcatggaccccatttttctcgccactgggtggcgtggatgctgggccggtcggagccccattgagcaaccctctgaaaaacccatcgcgtttgcccaattggatgaaaagaagattacggctaggactgtggtcgcccagccttatgatcccaaccaagccgtaaagtgcctgcgggtattgcaggcgggaggggcgatggtggctgaggcggtcccaaaagtggtcagggtttccgctgttccatttcgagcccccttttttcctaccggagtgaaggttgaccctgaatgtagagtagtggttgaccctgacactttcactgcagctctccggtctggttactccaccacaaacctcgtcctcggtgtgggggactttgcccagctgaatggattaaaaatcaggcaaatttctaagccttcaggggggggcccacatctcatggctgccctgcatgttgcctgctcgatgattttgcacatgtttgctgggatttatgtgactgcggtgggttcttgcggcaccggcaccaacgatccgtggtgcgcaaacccatttgccgtccctggctacggacctggctccctctgcacgtccagattgtgtatttcccagcacggccttaccctgcccttgacagcacttgtggcgggattcggcatccaggaaattgccttagtcgctttgatctttgtttccatcggaggcattgctcataggttgagttgtaaagctgacatgctgtgtgttttgcttgcaattgccagctatgtttgggtacctcttacctggttactttgtgtttttccttgctggttgcgctggttttctttgcatccccttaccgtcctatggttggtgtttttcttgatttctgtgaatatgccttcaggaatcttggccatggtgttgttagtttctctttggcttcttggtcgttatactaatgtcgccggtcttgtcactccctacgacattcatcattacaccagtggcccccgcggtgttgccgcattggctactgcaccagatggaacctacttagccgctgttcgccgcgctgcgttgactggccgcaccatgttgttcaccccgtcccagcttgggtctcttcttgagggcgctttcagaactcgaaagccctcactgaacaccgtcaatgtggtcgggtcctccatgggctctggcggggtgtttaccattgacgggaaagtcaggtgcgtgactgccacacatgtccttacgggtaactcagccagggtttccggaaccggcttcaatcaaatgcttgactttgacgtaaaaggggatttcgctatagccgattgcccgaattggcaaggggctgcccccaaaacccaattctgcgaggatggatggactggccgtgcctattggctaacatcctctggcgtcgaacccggcgtcattgggaaaggattcgccttctgcttcaccgcgtgcggcgattccgggtccccagtgatcaccgaggccggtgagcttgtcggtgttcacacgggatcgaataaacaaggggggggtattgtcacacgcccctcaggccagttttgtaatgtggcacccatcaagctgagcgaattaagtgaattctttgctgggcctaaggtcccgctcggtgatgtgaaggttggcagccacataatcaaagacacaagcgaggtgccttcagatctttgtgccttgcttgctgccaaacctgaactggaaggaggtctctccaccgtccaacttctgtgtgtgtttttccttctgtggagaatgatgggacatgcctggacgcccttggttgctgtgggtttctttattttgaatgagattctcccggccgtcctggtccggagtattttctcctttggaatgtttgtgctatcctggctcacgccatggtctgcgcaagttctgatgatcaggctcctgacagcagctcttaacaggaatagatgttcacttgcctttttcagcctcggtgcagtgaccggttttgttgcagatcttgcggctactcaggggcatccgttgcaggcagtgatgaatttgagcacctatgcattcctgcctcgggtaatggttgtgacttcaccagtcccagtgatcgcgtgtggtgttgtgcacctactcgccatcattctgtacctgtttaaataccgtagcctgcaccatatccttgttggcgatggagtgttctcttcggccttcttcctgcgatattttgccgaaggaaagttgagggaaggggtgtcacaatcctgcggaatgaatcatgagtctctgactggtgccctcgctatgagactcaatgacgaggacttggatttcctcatgaaatggactgattttaagtgcttcgtttctgcgtccaacatgaggaatgcagcgggtcaatttatcgaggctgcatatgctaaagcacttagagttgaactggcccagttggtgcaggttgataaggttcgaggtactatggccaaacttgaagcttttgctgatactgtggcacctcaactctcgcccggtgatattgttgtcgctctcggccacacgcctgttggcagtatcttcgacttaaaggttggtagcaccaagcataccctccaagccattgagaccagggttcttgctgggtccaaaatgaccgtggcgcgcgtcgtcgacccgacccccacgcccccacccgcgcccgtgcccatccccctcccaccgaaagttctggagaatggccccaacgcttggggggatgaggaccgtttgaataagaagaaaagacgcaggatggaagccctcggcatctatgttatgggcgggaaaaagtaccagaaattttgggacaagaactccggtgatgtgttttatgaggaggtccataacaacacagatgagtgggagtgtctcacagttggcgaccctgccgactttgaccctgagaagggaaccctgtgtggacatgtcaccattgacaacaaggcttaccatgtttacacctcctcatctggtaagaagttcttggttcccgtcaacccagagaacggaagagttcaatgggaagctgcaaagctttccgtggagcaggccctaggcatgatgaatgtcgacggcgaactgactgccaaggaactggagaaactgaaaagaataattgacaaactccagggcctgactaaggagcagtgtttaaactgctagccgccagcgacttgacccgctgtggtcgcggcggcttggttgttactgagacagcggtgaaaatagtcaaatttcacaatcggactttcactctggggcctgtgaatttaaaagtggccagtgaggttgagctaaaagatgcggttgagcacaaccaacacccggttgcgaggccgatcgatggtggagttgtgctcctgcgttccgcggttccctcgcttatagacgtcttgatctccggtgctgacacatctcccaagttacttgcccttcacgggccgggaaacactgggatcgatggcacgctctgggatttcgagtccgaagccactaaagaggaagtcgcactcagtgcgcaaataatacaggcttgtgacattaggcgtggcgacgctcctgaaattggtctcccttataagctgtaccctgttaggggcaaccctgagcgggtgaaaggagttctgcagaatacaaggtttggagacataccctacaaaacccctagtgacaccgggagcccagtgcacgcggctgcctgccttacgcccaacgccactcctgtgactgatgggcgttccgtcttggctacaaccatgccccccgggtttgagttatatgtaccgaccataccagcgtctgtccttgattaccttgactctaggcctgactgccctaaacagctgacagagcacggctgcgaagacgccgcactgaaagacctctctaaatatgacttatctacccaaggctttgtcttacctggggttcttcgccttgtgcggaaatacctgtttgcccatgtaggtaaatgtccacccgttcatcggccttctatttaccccgctaagaattctatggctggaataaatgggaacaggttcccaaccaaagacatccagagcgttcctgaaatcgacgttctgtgcgcacaggctgtgcgggaaaactggcaaactgtcaccccttgtactcttaagaaacagtattgcgggaagaagaagaccaggaccatactcggcaccaataacttcattgcactggcccaccgagcagtgttgagtggtgtcacccagggcttcatgaaaaaggcgttcaactcgcccatcgccctcggaaagaacaagttcaaggagctacagactccggtcctgggcaggtgccttgaagctgacctcgcatcctgtgatcgatccacgcctgcaattgttcgctggtttgctgccaaccttctttatgaacttgcctgtgctgaagagtatctaccgtcgtatgtgctgaactgctgccacgacttactagtcacgcagtccggcgcagtgactaagagaggtggcctgtcgtctggcgacccgatcacctctgtgtctaacaccatttacagtttggtgatctatgcacagcacatggtgcttagttactttaaaagtggtcacccccatggccttctgttcttacaagaccagctaaagtttgaggacatgctcaaggttcaacccctgatcgtctattcggacgatctcgtgctgtatgccgagtctcccaccatgccaaattatcactggtgggttgagcatctgaatttgatgctggggtttcagacggacccaaaaaaaacagcaataacagattcgccatcatttctgggctgtagaataatgaatgggcgccagctagtccccaaccgtgacaggatcctcgcggccctcgcctatcacatgaaggcaagtaatgtttctgaatactatgcctcagcggctgcaatactcatggacagctgtgcttgtttggagtatgatcctgaatggtttgaagaacttgtagttggaatagcgcagtgcgcccgcaaggatggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatctaccacacccacttccaccagcattgtccagtcacaatctggtgtggccatccagcgggttctggttcttgtagtgagtgcaaatcccctgtagggaaaggcacaaaccctttagacgaggtgctggaacaagtcccgtataagcccccacggaccgttatcatgcatgtagagcagggtctcaccccccttgatccaggtagataccaaactcgccgcggattagtttccgttaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgcagggaaaacatactggctccttcaacaggtccaggacggtgatgttatttacacaccaactcaccagaccatgcttgacatgatcagggctttggggacgtgccggttcaacgtcccggcaggcacaacgctgcaattccctgtcccctcccgcaccggtccgtgggttcgcatcctggccggcggttggtgtcctggcaagaattccttcctagatgaagcagcgtattgcaaccaccttgatgttttgaggcttcttagcaaaactaccctcacctgtctaggagacttcaagcaactccacccagtgggttttgattctcattgttatgtttttgacatcatgcctcaaactcaactgaagaccatctggaggttcggacagaatatctgtgatgccatccagccagattacagggacaagctcatgtccatggtcaacacaacccgtgtgacctacgtggaaagacctgtcaggtatgggcaggtcctcaccccctaccacagggaccgagaggacgacgccatcactattgactccagtcaaggcgccacattcgatgtggtcacattgcatttgcccactaaagattcactcaacaggcaaagagcccttgttgccatcaccagggctagacacgctatctttgtgtatgacccacacaggcagctgcagagcttgtttgatcttcctgcaaaaggcacacccgtcaaccttgcagtgcaccgcgacgggcagctgatcgtacttgatagaaataacaaagaatgcacggttgctcaggctctaggcaacggggataaattcagggccacagacaagcgtgttgcagattcactccgcgccatttgtgctgatctagaagggtcgagttctccgctccccaaggtcgcacacaacttggggttttatttctcacctgacttaacacaatttgccaaactcccagtggaacttgcacctcattggcccgtggtgacaacccagaacaatgaaaagtggccagatcggctggtcgccagccttcgccctatccataaatacagccgcgcgtgcatcggtgccggctatatggtgggcccttcggtgtttctaggcactcctggggtcgtatcatactttcttacaaaatttgttaaaggcgaggctcaaatgcttccagaaacagtcttcagcaccggccgaattgaggtggattgccgggaatatcttgacgatcgggagcgggaagttgctgcatccctcccacacgcctttattggcgacgtcaaaggcaccaccgttggcggatgtcatcatgtcacctccaggtaccttcctcgcttccttcccaaagagtcagttgcggtagtcggggtctcaagccccgggaaagccgcgaaggcaatgtgcacactgacagatgtgtacctcccagatcttgaagcctatctccacccggagacccagtccaggtgctggaaaatgatgttggacttcaaagaagttcgactgatggtctggaaagacaaaacggcctatttccaacttgagggtcgttatttcacctggtatcagctcgctagttatgcctcgtacgtccgtgttcctgtcaactccacggtgtacttggacccctgcatgggcccagcccattgcaacagacgagttgtcgggtccactcagtggggagctgacctcgcaatcaccccctatgattatggcgccaagatcattttgtctagcgcatatcacggtgaaatgccccctgggtacaagattctggcgtgtgcggagttctcattggatgacccggtcagatacaaacacacctggggatttgagtcggatacagcgtacctgtacgagttcactggaaatggtgaggattgggaagattataatgatgcgtttcgtgcgcgccagaaagggaaaatttataaggccactgccaccagcttgaagttttatttccctccgggccctgtcgttgaaccaactttgggcctgaactgaaggcgcgccgccaccatgctgtggggcgtatattccgcaatcgaaacgtggaagttcataacgagtcgctgccgtctctgcttattagggcgaaagtatatactcgctcctgctcaccacgtggagtcggcttaggttccgtggcaacccctataaccagagtttcagcggaacaggcgcgccatgaaatggggtccatgcaaagccttttttacaaaattggccaactttttgtggatgctttcacggagttcttggtgtccattgttgatatcattatatttttggccattttgtttggcttcaccatcgccggttggctggtggtcttttgcatcagattggtttgctccgcgatactccgtacgcgccctgccattcactctgagcaattacagaagatcttatgaggcctttctttcccagtgccaagtggacattcccacctggggaactaaacatcctttggggatgctttggcaccataaggtgtcaaccctgattgatgaaatggtgtcgcgtcgaatgtaccgcatcatggaaaaagcagggcaggctgcctggaaacaggtggtgagcgaggctacgctgtctcgcattagtagtttggatgtggtggctcattttcagcatctagccgccattgaagccgagacctgtaaatatttggcctcccggctgcccatgctacacaacctgcgcatgacaggttcaaatgtaaccatagtgtataatagcactttgaatcaggtgtttgctatttttccaacccctggttcccggccaaagcttcatgattttcagcaatggttaatagctgtacattcctccatattttcctctgttgcagcttcttgtactctttttgttgtgctgtggttgcgggttccaatactacgtactgtttttggtttccgctggttaggggcaatttttctttcgaactcacagtgaattacacggtgtgtccaccttgcctcacccggcaagcagccacagagatctacgaacccggtaggtctctttggtgcaggatagggtatgaccgatgtgaggaggatgatcatgacgagctagggtttatggtaccgcctggcctctccagcgaaggccacttgactagtgtttacgcctggttggcgttcttgtccttcagctacacggcccagttccatcccgagatattcgggatagggaatgtgagtcgagtttatgttgacatcaaacatcaactcatctgcgccgaacatgacgggcagaacaccaccttgcctcgtcatgacaacatttcagccgtgtttcagacctattaccaacatcaagtcgacggcggcaattggtttcacctagaatggcttcgtcccttcttttcctcgtggttggttttaaatgtctcttggtttctcaggcgttcgcctgcaaaccatgtttcagttcgagtcttgcagatattaagaccaacaccaccgcagcggcaagctttgctgtcctccaagacatcagttgccttaggcatcgcgactcggcctctgaggcgattcgcaaaatccctcagtgccgtacggcgatagggacacccgtgtatgttaccatcacagccaatgtgacagatgagaattatttacattcttctgatctcctcatgctttcttcttgccttttctatgcttctgagatgagtgaaaagggatttaaggtggtatttggcaatgtgtcaggcatcgtggctgtgtgtgtcaattttaccagctacgtccaacatgtcaaggagtttacccaacgctccctggtggtcgaccatgtgcggttgctccatttcatgacacctgagaccatgaggtgggcaactgttttagcctgtctttttgccattctgttggcaatttgaatgtttaagtatgttggagaaatgcttgaccgcgggctgttgctcgcaattgctttctttgtggtgtatcgtgccgttctgttttgctgtgctcgccaacgccagcaacgacagcagctcccatctacagctgatttacaacttgacgctatgtgagctgaatggcacagattggctagctaacaaatttgattgggcagtggagagttttgtcatctttcccgttttgactcacattgtctcctatggtgccctcactaccagccatttccttgacacagtcgctttagtcactgtgtctaccgccgggtttgttcacgggcggtatgtcctaagtagcatctacgcggtctgtgccctggctgcgttgacttgcttcgtcattaggtttgcaaagaattgcatgtcctggcgctacgcgtgtaccagatataccaactttcttctggacactaagggcggactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccagaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggtgagatttaattggcgagaaccatgcggccgaaattaaaaaaaaaaaaaaaaaaaaaaaaggccggcatggtcccagcctcctcgctggcgccggctgggcaacatgcttcggcatggcgaatgggacttaattaa

SEQ ID NO: 12 - protein, 38 aa, Artificial Sequence
  (Synthetic)_T cell epitope_EP8_Amino_Acid
LWGVYSAIETWKFITSRCRLCLLGRKYILAPAHHVESA

SEQ ID NO: 13 - DNA, 6 bp, Artificial Sequence
  (Synthetic)_Kozak sequence
gccacc

SEQ ID NO: 14 - DNA, 40 bp, Artificial Sequence
  (Synthetic)_TRS (transcription regulatory sequence) 6
gttccgtggcaacccctataaccagagtttcagcggaaca

SEQ ID NO: 15 - DNA, 27 bp, Artificial Sequence
  (Synthetic)_2-Vector-seq-FOR primer
gggaagattataatgatgcgtttcgtg

SEQ ID NO: 16 - DNA, 20 bp, Artificial Sequence
  (Synthetic)_PRRS-ORF2-REV primer
accagccaaccggcgatggt

SEQ ID NO: 17 - DNA, 18 bp, Artificial Sequence
  (Synthetic)_PRRSV specific Primer For
atgatgrgctggctaact

SEQ ID NO: 18 - DNA, 19 bp, Artificial Sequence
  (Synthetic)_PRRSV specific Primer Rev
acaccggtcgccctaattg

SEQ ID NO: 19 - DNA, 24 bp, Artificial Sequence
  (Synthetic)_Taq man Probe
tgtggtgaatggcactgattgaca

SEQ ID NO: 20 - DNA, 146 bp, Artificial Sequence
  (Synthetic)_Modified T cell epitope-EP7
ggcgcgccgccaccatgctgctagctttcagtatcacatatactccagttatgatatacgcacttaaggtctctcgtgggcgtctcctagggctctaggttccgtggcaacccctataaccagagtttcagcggaacaggcgcgcc

SEQ ID NO: 21 - DNA, 182 bp, Artificial Sequence
  (Synthetic)_Modified T cell epitope-EP8
ggcgcgccgccaccatgctgtggggcgtatattccgcaatcgaaacgtggaagttcataacgagtcgctgccgtctctgcttattagggcgaaagtatatactcgctcctgctcaccacgtggagtcggcttaggttccgtggcaacccctataaccagagtttcagcggaacaggcgcgcc